(19) United States
(12) Patent Application Publication
(10) Pub. No.: US 2017/0242969 A1
(43) Pub. Date: Aug. 24, 2017

(54) CAPTURING AND MANAGING HEALTHCARE INFORMATION

(71) Applicant: CAPSULE TECH, INC., Andover, MA (US)

(72) Inventors: Rémy BAUDET, Boston, MA (US); Andrew WESCOTT, Stoneham, MA (US)

(21) Appl. No.: 15/504,747

(22) PCT Filed: Sep. 14, 2015
(86) PCT No.: PCT/US15/49899

§ 371 Date: Feb. 17, 2017

Related U.S. Application Data
(60) Provisional application No. 62/050,398, filed on Sep. 15, 2014.

(51) Int. Cl. G06F 19/00; A61B 5/00

(52) U.S. Cl. CPC G06F 19/3412; G06F 19/322; A61B 5/7435; G06F 21/6245

(57) ABSTRACT

Methods, systems, computer-readable media, and apparatuses for capturing and managing healthcare information are presented. In one or more embodiments, a server computing device may establish a first network connection to a first bedside computing device. Subsequently, the server computing device may select a first device profile for the first bedside computing device. The server computing device then may provide the first device profile to the first bedside computing device. Thereafter, the server computing device may establish a second network connection to a second bedside computing device different from the first bedside computing device. Subsequently, the server computing device may select a second device profile for the second bedside computing device.

### CROSS-REFERENCE TO RELATED APPLICATIONS

This application claims the benefit of and priority to U.S. Provisional Patent Application Ser. No. 62/050,398, filed Sep. 15, 2014, and entitled “CAPTURING AND MANAGING HEALTHCARE INFORMATION,” which is incorporated by reference herein in its entirety.

### BACKGROUND

Aspects of the disclosure relate to computing hardware and computer software. In particular, one or more aspects of the disclosure are directed to computing hardware and computer software for capturing and managing healthcare information.

Healthcare organizations are increasingly adopting and deploying many different types of information systems across many different environments. For example, a healthcare organization, such as a hospital or hospital system, may utilize enterprise-wide information systems to electronically manage patient records and/or other information, specialized clinical systems to maintain information that is particularly relevant to different clinics, and/or other ancillary systems that may support laboratory functions, such as radiology functions, pharmacy functions, and/or other clinical functions.

As different information systems and medical devices are adopted and used by a healthcare organization, it may be difficult for the organization to connect all of the various information systems in a way that allows individual healthcare providers to monitor and analyze information that may be captured and/or maintained by different information systems and subsequently make informed decisions and/or otherwise act on such information in an effective manner.

### SUMMARY

Aspects of the disclosure relate to various systems and techniques that provide efficient, effective, scalable, and convenient ways of capturing and managing healthcare information, particularly in ways that enable a healthcare organization to centrally and uniformly manage information that may be captured and/or maintained by different information sources and/or systems. As illustrated below, by implementing various aspects of the disclosure, a healthcare organization and/or a healthcare provider associated with such a healthcare organization may be able to obtain a clear, real-time view of pertinent healthcare information, such as patient vital signs information, that enables the organization and/or the provider to quickly understand and act on such information in an efficient and effective manner.

In some embodiments, one or more bedside computing devices may be configured by a central server which may manage multiple bedside computing devices in a patient care environment, such as a hospital, and which may interface with other information systems used by a healthcare organization, such as an electronic medical records (EMR) system. For example, in accordance with one or more embodiments, a server computing device may establish a first network connection to a first bedside computing device. Subsequently, the server computing device may select a first device profile for the first bedside computing device. The server computing device then may provide the first device profile to the first bedside computing device. Thereafter, the server computing device may establish a second network connection to a second bedside computing device different from the first bedside computing device. Subsequently, the server computing device may select a second device profile for the second bedside computing device. The server computing device then may provide the second device profile to the second bedside computing device.

In other embodiments, a bedside computing device may capture and process sensor data which may then be provided to a central server. As above, such a central server may, for instance, manage multiple bedside computing devices in a patient care environment and/or may interface with other information systems used by a healthcare organization. For example, in accordance with one or more embodiments, a bedside computing device may capture sensor data received from one or more sensors connected to the bedside computing device. Subsequently, the bedside computing device may process the sensor data to determine patient vital signs information. The bedside computing device then may provide the patient vital signs information to a server computing device that is configured to receive vital signs information from one or more additional bedside computing devices.

In other embodiments, a central server may remotely manage multiple different bedside computing devices that may be deployed in a patient care environment. As above, such a central server may, for instance, interface with other information systems used by a healthcare organization, such as an EMR system. For example, in accordance with one or more embodiments, a server computing device may receive input defining a first device profile for a first set of one or more bedside computing devices. Subsequently, the server computing device may establish a network connection to at least one bedside computing device included in the first set of one or more bedside computing devices. The server computing device then may provide the first device profile to the at least one bedside computing device.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIG. 1 depicts an example of a computing device in accordance with one or more illustrative aspects of the disclosure.

FIG. 2 depicts an example of a computing environment for capturing and managing healthcare information in accordance with one or more illustrative aspects of the disclosure.

FIGS. 3A-3D depict an example of an event sequence for configuring a plurality of bedside computing devices in accordance with one or more illustrative aspects of the disclosure.

FIGS. 4A-4D depict an example of an event sequence for capturing and processing sensor data in accordance with one or more illustrative aspects of the disclosure.

FIGS. 5A-5E depict an example of an event sequence for managing a plurality of bedside computing devices in accordance with one or more illustrative aspects of the disclosure.

FIGS. 6-30 depict examples of graphical user interfaces that may be presented in capturing and processing sensor data in accordance with one or more illustrative aspects of the disclosure.

FIGS. 31-33 depict examples of graphical user interfaces that may be presented in configuring and managing a plurality of bedside computing devices in accordance with one or more illustrative aspects of the disclosure.

FIG. 34 depicts an example of a method of configuring a plurality of bedside computing devices in accordance with one or more illustrative aspects of the disclosure.

FIG. 35 depicts an example of a method of capturing and processing sensor data in accordance with one or more illustrative aspects of the disclosure.

FIG. 36 depicts an example of a method of managing a plurality of bedside computing devices in accordance with one or more illustrative aspects of the disclosure.

FIGS. 37 and 38 depict additional examples of computing environments for capturing and managing healthcare information in accordance with one or more illustrative aspects of the disclosure.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. It is to be understood that other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

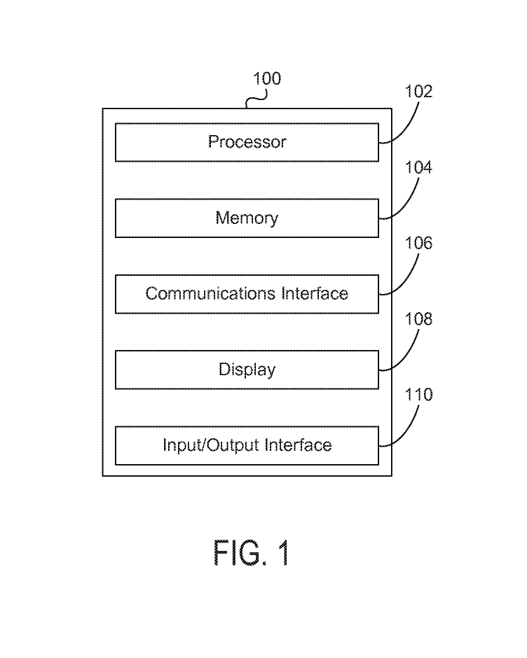

FIG. 1 depicts an example of a computing device in accordance with one or more illustrative aspects of the disclosure. As seen in FIG. 1, computing device 100 may include a processor 102, memory 104, communications interface 106, display 108, and an input/output (I/O) interface 110. Processor 102 may control various operations of the computing device 100, which may include controlling and/or otherwise interacting with various components of the computing device, including memory 104, communications interface 106, display 108, and/or I/O interface 110. Memory 104 may store computer-readable instructions that may, for example, be executed by processor 102. In addition, memory 104 may store applications and/or other data that may be used in implementing various aspects of the disclosure. Communications interface 106 may include one or more wired communication interfaces (e.g., an Ethernet interface), one or more wireless communication interfaces (e.g., a wireless local area networking (WLAN) interface, a Bluetooth interface, etc.), and/or one or more other communication interfaces. Communications interface 106 may, for example, enable computing device 100 to exchange information and/or electronic signals with one or more other computing devices via one or more communication networks or connections. Display 108 may include a video display unit via which computing device 100 may provide image and/or video output. In some instances, display 108 may include a touch-sensitive surface that may receive touch-based input (e.g., from a user of computing device 100 provided by a stylus, the user's finger, etc.). I/O interface 110 may include a camera, a microphone, an audio speaker, one or more ports, and/or other components that may enable computing device 100 to receive various types of input and/or provide various types of output.

Computing device 100 provides an example of a computing device that may be used in implementing various aspects of the disclosure. For example, one or more of the computing platforms, servers, and/or other computing devices discussed below may incorporate one or more aspects of computing device 100. As illustrated in greater detail below, while one or more of the computing platforms, servers, and/or other computing devices discussed below may incorporate one or more aspects of computing device 100, these computing platforms, servers, and/or other computing devices may be special-purpose computing devices that are configured to perform specific and special-purpose computer functions. In addition, while computing device 100 provides one example arrangement of a computing device, one or more aspects of the disclosure may be similarly implemented in computing devices having other arrangements. For example, in some alternative arrangements, a computing device may include one or more additional and/or alternative components in addition to and/or instead of those discussed here. For instance, in some alternative arrangements, a computing device may include multiple instances of the components shown in FIG. 1 and/or other components (e.g., two or more processors, two or more memories, two or more displays, etc.).

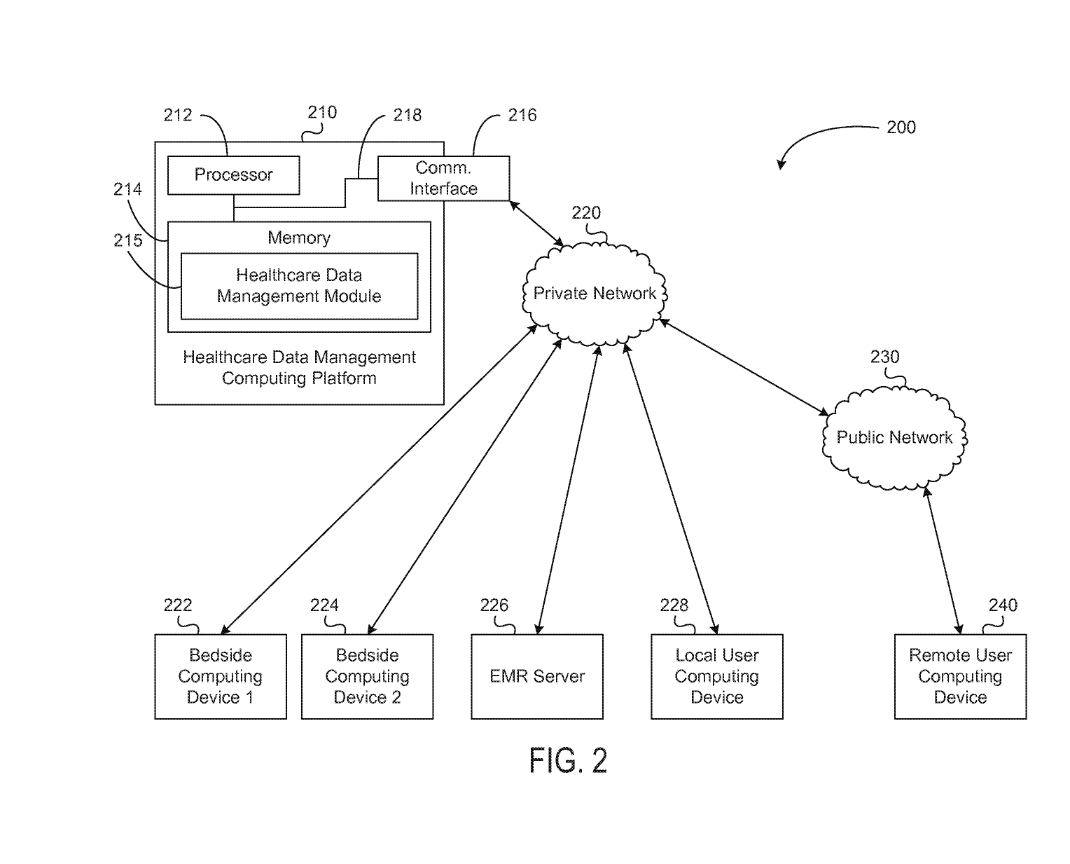

FIG. 2 depicts an example of a computing environment for capturing and managing healthcare information in accordance with one or more illustrative aspects of the disclosure. As seen in FIG. 2, computing environment 200 may include a first bedside computing device 222 (which may, e.g., be used by a healthcare organization and/or a healthcare provider in a first patient care location, as discussed in greater detail below), a second bedside computing device 224 (which may, e.g., be used by such a healthcare organization and/or healthcare provider in a second patient care location), an electronic medical records (EMR) server 226 (which may, e.g., be configured to maintain and/or manage electronic medical records for one or more patients of the healthcare organization and/or healthcare provider, as discussed in greater detail below), a local user computing device 228 (which may, e.g., be used by an administrative user of a healthcare information system, as discussed in greater detail below), and a remote user computing device 240 (which may, e.g., be used by another user of such a healthcare information system, as discussed in greater detail below). Bedside computing device 222, bedside computing device 224, EMR server 226, user computing device 228, and/or user computing device 240 may incorporate one or more aspects of computing device 100 and may be any type of computing device capable of receiving a user interface, receiving input via the user interface, and communicating the received input to one or more other computing devices. For example, bedside computing device 222, bedside computing device 224, EMR server 226, user computing device 228, and/or user computing device 240 may be a desktop computer, laptop computer, tablet computer, smart phone, or the like. As discussed in greater detail below, bedside computing device 222 and bedside computing device 224 may, in some arrangements, be specialized devices that may be specifically configured for use in a healthcare environment. For instance, bedside computing device 222 and bedside computing device 224 may include various ports and/or specialized software that provide connectivity with various medical sensors, such as blood pressure sensors, pulse sensors, oxygen saturation sensors, pulse oximeter sensors, temperature sensors, and/or other sensors. Additionally or alternatively, bedside computing device 222 and bedside computing device 224 may include various ports and/or specialized software that provide connectivity with various medical devices, such as cardiac monitors, infusion pumps, and/or other medical devices. In some arrangements, bedside computing device 222 and bedside computing device 224 may be medical-grade devices and accordingly may meet and/or exceed one or more standards that may be defined for such devices. For example, bedside computing device 222 and bedside computing device 224 may include one or more USB ports and/or one or more serial ports (which may, e.g., enable one or more sensors and/or medical devices to be connected to bedside computing device 222 and bedside computing device 224), and each of the one or more USB ports and/or each of the one or more serial ports may be electrically isolated.

Computing environment 200 also may include one or more computing platforms. For example, computing environment 200 may include healthcare data management computing platform 210. Healthcare data management computing platform 210 may include one or more computing devices configured to perform one or more of the functions described herein. For example, healthcare data management computing platform 210 may operate as and/or include one or more server computing devices which may, for instance, incorporate one or more aspects of computing device 100 and/or which may, for instance, configure, communicate with, and/or manage one or more bedside computing devices, such as bedside computing device 222 and/or bedside computing device 224, as discussed in greater detail below.

Computing environment 200 also may include one or more networks, which may interconnect one or more of healthcare data management computing platform 210, bedside computing device 222, bedside computing device 224, EMR server 226, user computing device 228, and remote user computing device 240. For example, computing environment 200 may include a private network 220 and a public network 230. Private network 220 and/or public network 230 may include one or more sub-networks (e.g., local area networks (LANs), wide area networks (WANs), virtual private networks (VPNs), etc.). Private network 220 may be operated by and/or otherwise associated with a particular organization (e.g., a corporation, partnership, or the like) and may interconnect one or more computing devices associated with the organization. For example, healthcare data management computing platform 210, bedside computing device 222, bedside computing device 224, EMR server 226, and local user computing device 228 may be owned by, operated by, and/or otherwise associated with a healthcare organization (which may, e.g., deploy and/or use any and/or all of such computing systems in a patient care environment, such as a hospital system, hospital, clinic, etc.), and private network 220 may be owned by, operated by, and/or otherwise associated with the healthcare organization, and may include one or more networks (e.g., LANs, WANs, VPNs, etc.) that interconnect healthcare data management computing platform 210, bedside computing device 222, bedside computing device 224, EMR server 226, and local user computing device 228. Public network 230 may connect private network 220 and/or one or more computing devices connected thereto (e.g., healthcare data management computing platform 210, bedside computing device 222, bedside computing device 224, EMR server 226, and/or local user computing device 228) with one or more networks and/or computing devices that are not associated with the organization. For example, user computing device 240 might not be associated with the organization that operates private network 220, and public network 230 may include one or more networks (e.g., the Internet) that connect user computing device 240 to private network 220 and/or one or more computing devices connected thereto (e.g., healthcare data management computing platform 210, bedside computing device 222, bedside computing device 224, EMR server 226, and/or local user computing device 228).

Healthcare data management computing platform 210 may include at least one processor 212, memory 214, communication interface 216, and data bus 218. Data bus 218 may interconnect processor 212, memory 214, and/or communication interface 216. Communication interface 216 may be a network interface configured to support communication between healthcare data management computing platform 210 and private network 220 or one or more sub-networks thereof. Memory 214 may include one or more program modules comprising instructions that, when executed by processor 212, cause healthcare data management computing platform 210 to perform one or more functions described herein. For example, memory 214 may include healthcare data management module 215, which may include instructions that, when executed by processor 212, cause healthcare data management computing platform 210 to perform data processing for capturing and managing healthcare data in accordance with various aspects of the disclosure.

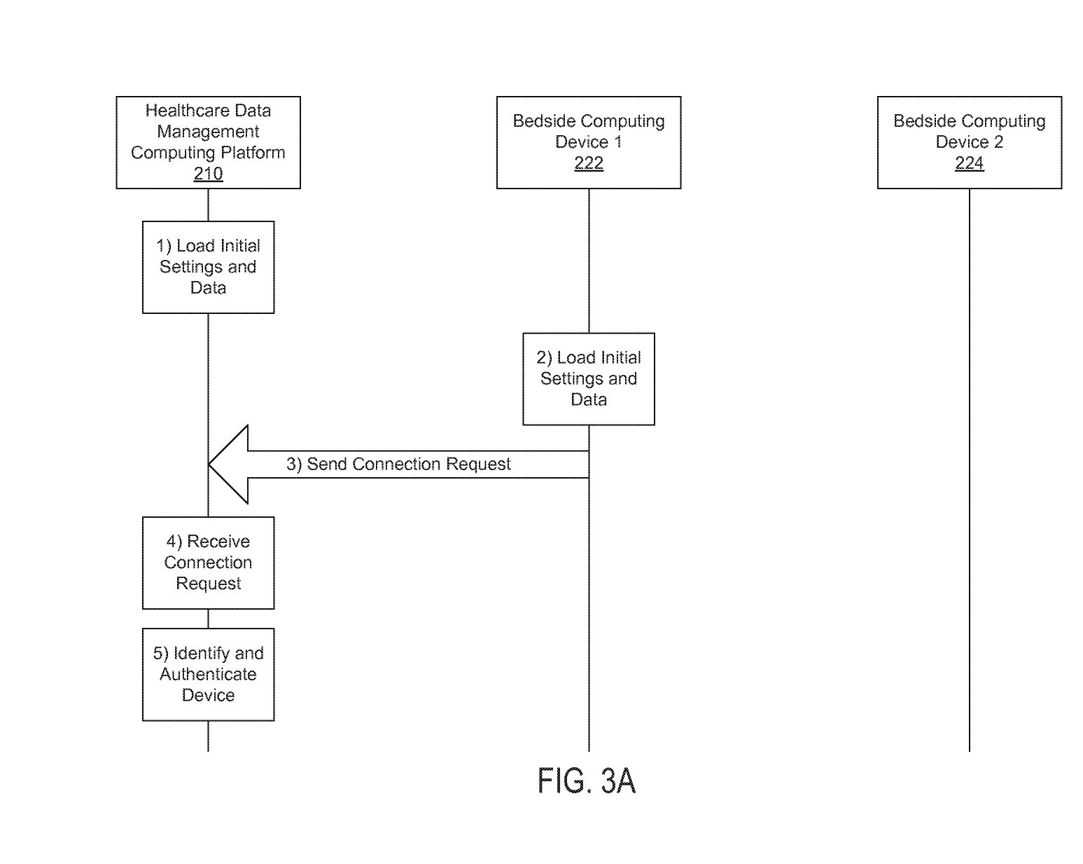

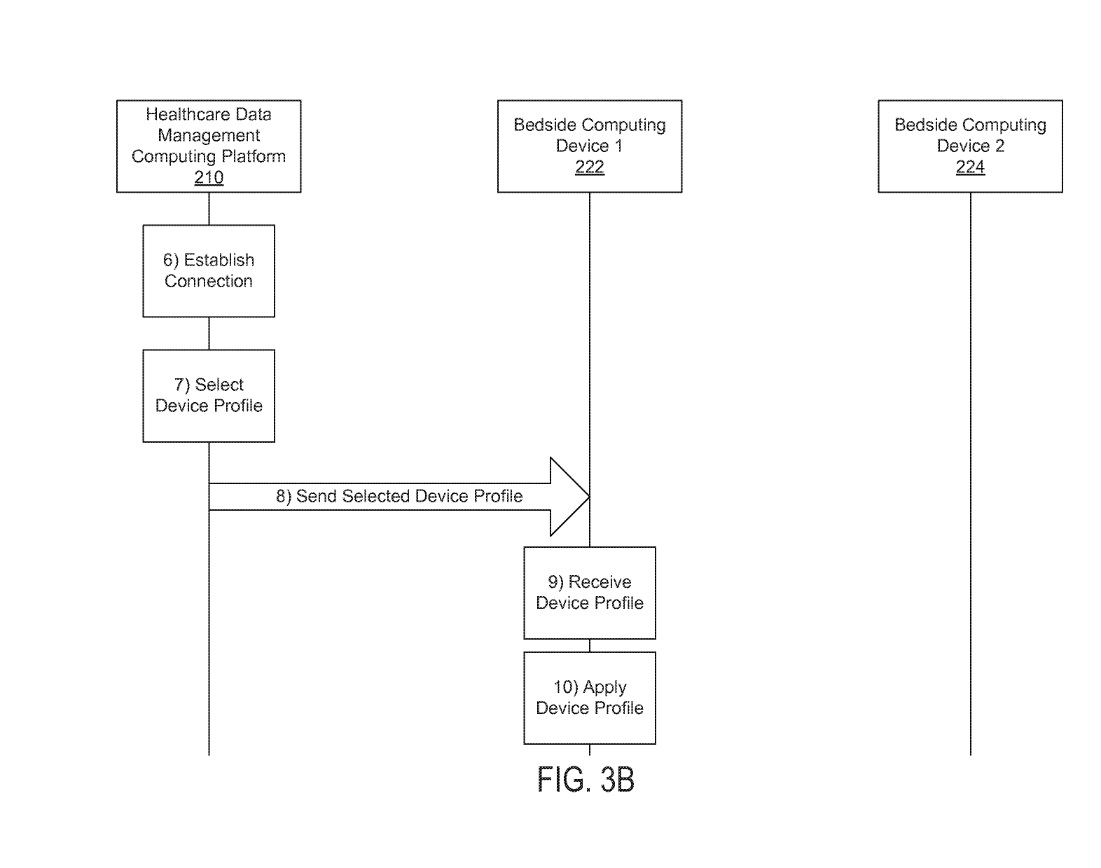

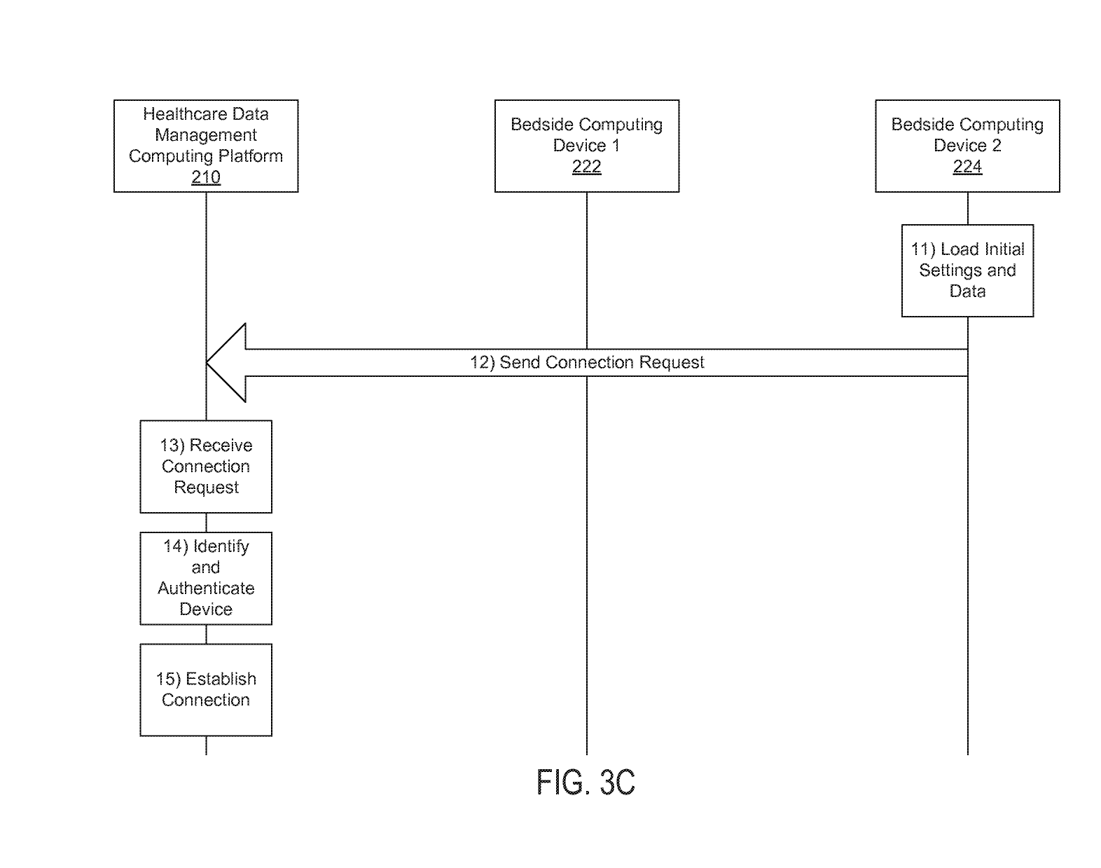

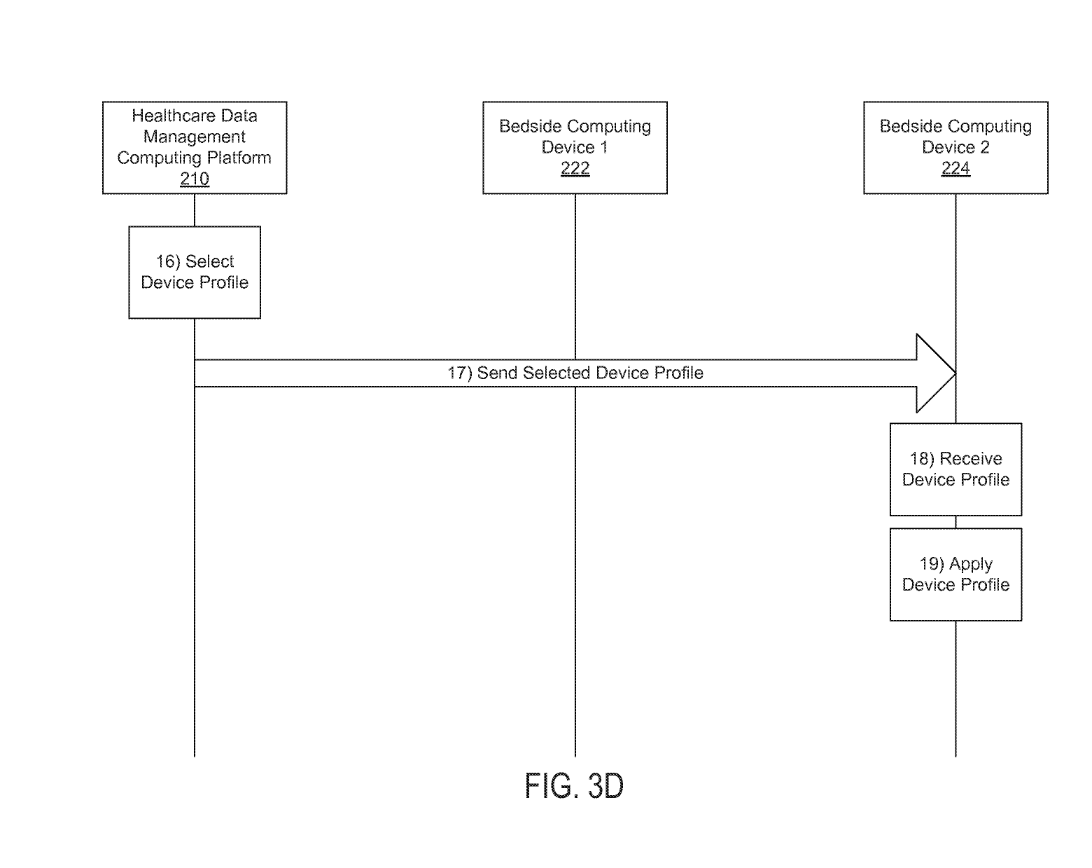

FIGS. 3A-3D depict an example of an event sequence for configuring a plurality of bedside computing devices in accordance with one or more illustrative aspects of the disclosure. Referring to FIG. 3A, at step 1, healthcare data management computing platform 210 may load initial settings and data. Such initial settings and data may, for instance, include default network settings, security settings, data capture and/or processing settings, and/or other settings that may be used by healthcare data management computing platform 210 in performing various functions. In some instances, healthcare data management computing platform 210 may load such initial settings and data after being powered on and/or otherwise initialized. At step 2, bedside computing device 222 may load initial settings and data. For example, bedside computing device 222 may load default network settings, security settings, data capture and/or processing settings, and/or other settings that may be used by bedside computing device 222 in performing various functions. In some instances, bedside computing device 222 may load such initial settings and data after being powered on and/or otherwise initialized. Additionally or alternatively, the initial settings and/or data that may be loaded by bedside computing device 222 when initially starting up (e.g., at step 2) include one or more settings and/or data that correspond to and/or are otherwise associated with the last good configuration of the bedside computing device 222 (which may, e.g., be periodically backed up and/or saved by bedside computing device 222, as discussed in greater detail below). By using such settings and/or data (which may, e.g., correspond to the last good configuration of bedside computing device 222), bedside computing device 222 may be able to provide various functionalities and/or otherwise be used by a user of bedside computing device 222 even if a connection to one or more servers, such as healthcare data management computing platform 210, cannot be established. At step 3, bedside computing device 222 may send a connection request to healthcare data management computing platform 210. Such a connection request may, for instance, be sent as a result of, in accordance with, and/or otherwise based on one or more of the initial settings loaded by bedside computing device 222.

At step 4, healthcare data management computing platform 210 may receive a connection request from a bedside computing device. For example, healthcare data management computing platform 210 may receive the connection request sent by bedside computing device 222. At step 5, healthcare data management computing platform 210 may identify and authenticate the bedside computing device (e.g., bedside computing device 222). In identifying and authenticating the bedside computing device, healthcare data management computing platform 210 may, for example, request, receive, and/or validate one or more unique device identifiers associated with bedside computing device 222, one or more network addresses associated with bedside computing device 222, location information associated with bedside computing device 222 (which may, e.g., specify a deployment location of the bedside computing device), and/or one or more credentials and/or tokens associated with bedside computing device 222.

Referring to FIG. 3B, at step 6, healthcare data management computing platform 210 may establish a first network connection to a first bedside computing device. For example, after identifying and/or authenticating bedside computing device 222 (e.g., at step 5), healthcare data management computing platform 210 may establish a network connection with bedside computing device 222 (e.g., at step 6). At step 7, healthcare data management computing platform 210 may select a first device profile for the first bedside computing device. For example, at step 7, healthcare data management computing platform 210 may select a device profile for bedside computing device 222. Such a device profile may, for instance, be selected by healthcare data management computing platform 210 for bedside computing device 222 from a set of predefined device profiles based on one or more factors.

In some embodiments, the first device profile for the first bedside computing device may be selected based on a unique identifier associated with the first bedside computing device. For example, in selecting a device profile for the first bedside computing device, healthcare data management computing platform 210 may select a particular device profile from a set of one or more predefined device profiles based on a unique device identifier that has been assigned to and/or otherwise associated with the first bedside computing device (e.g., bedside computing device 222).

In some embodiments, the first device profile for the first bedside computing device may be selected based on a deployment location of the first bedside computing device. For example, in selecting a device profile for the first bedside computing device, healthcare data management computing platform 210 may select a particular device profile from a set of one or more predefined device profiles based on a deployment location of the first bedside computing device (e.g., bedside computing device 222). Such a deployment location may, for instance, be identified and/or otherwise determined by healthcare data management computing platform 210 based on a network address associated with the first bedside computing device (e.g., bedside computing device 222). Additionally or alternatively, the deployment location of the first bedside computing device may be defined by location information that is accessed by and/or received by healthcare data management computing platform 210, and such location information may, for instance, specify a particular location in a patient care environment where the first bedside computing device (e.g., bedside computing device 222) is deployed, such as a particular clinic room, a particular operating room, a particular floor of a hospital, and/or the like.

In some embodiments, the first device profile for the first bedside computing device may be configured to cause the first bedside computing device to process sensor data received from at least one sensor that is directly connected to the first bedside computing device. For example, the first device profile (which may, e.g., be selected by healthcare data management computing platform 210 for bedside computing device 222) may be configured to cause bedside computing device 222 to process sensor data that is received from one or more sensors that are directly connected to bedside computing device 222. In some instances, one or more sensors that are directly connected to the bedside computing device may be connected via a sensor module (which may, e.g., include a relatively small circuit board and/or other electronics) that provides a serial adapter and/or universal serial bus (USB) adapter for the one or more sensors. Such sensors then may, for instance, be connected to a serial port and/or a USB port included in and/or coupled to the bedside computing device. Whether or not such directly connected sensors are connected to the bedside computing device via such a sensor module, the one or more directly connected sensors may provide raw signal data (e.g., analog signal data) to the bedside computing device. In addition, the bedside computing device may, for instance, process such raw signal data to determine and/or otherwise acquire one or more values associated with one or more conditions being monitored by the one or more directly connected sensors. For example, a blood pressure sensor may provide raw analog signal data to the bedside computing device when it is directly connected to the bedside computing device, and the bedside computing device may calculate, determine, and/or otherwise acquire a blood pressure value based on the raw analog signal data received from the blood pressure sensor.

In some instances, one or more sensors may be directly connected to a bedside computing device via one or more wireless connections. For example, one or more sensors may directly connect to the bedside computing device via a Bluetooth connection and/or may pair with the bedside computing device accordingly. Additionally or alternatively, one or more wearable sensors may be attached to and/or worn by a patient and may directly connect to the bedside computing device using one or more wireless connections. Such a wearable sensor may, for instance, be configured to wirelessly provide sensor data to the bedside computing device when the patient and/or the wearable sensor is located within a predetermined distance of the bedside computing device (e.g., a relatively short distance such as within five to ten meters).

In some embodiments, the first device profile for the first bedside computing device may be configured to cause the first bedside computing device to process sensor data received from at least one sensor that is connected to the first bedside computing device via at least one medical device. For example, the first device profile (which may, e.g., be selected by healthcare data management computing platform 210 for bedside computing device 222) may be configured to cause bedside computing device 222 to process sensor data that is received from one or more sensors that are connected to bedside computing device 222 via one or more medical devices and/or that are otherwise indirectly connected to bedside computing device 222. As illustrated below, such a device profile may, for instance, include various information that is created and/or defined by healthcare data management computing platform 210, and the information that is created and/or defined by healthcare data management computing platform 210 and included in the device profile may cause the bedside computing device (e.g., bedside computing device 222) to perform one or more specific functions in one or more specific ways, such as processing sensor data from one or more sensors in one or more specific ways. In particular such a medical device may, for instance, receive and/or process raw signal data from one or more sensors (e.g., to determine one or more values associated with one or more conditions being monitored by the one or more sensors) and subsequently may provide the processed data to the bedside computing device. The processed data may, for instance, be provided by the medical device to the bedside computing device as digital data. The bedside computing device may then present and/or otherwise use the processed data, but might not need to further process and/or analyze the data received from the medical device, since the values associated with the monitored conditions may have been already determined by the medical device. For example, a pulse oximeter sensor may be indirectly connected to the bedside computing device via a medical device, and the medical device may receive raw analog signal data from the pulse oximeter sensor. Subsequently, the medical device may process the raw analog signal data to determine values for the pulse rate and the oxygen saturation being monitored by the sensor, and the medical device may provide digital information to the bedside computing device that includes the determined pulse rate and oxygen saturation values.

In some embodiments, the first device profile may include location information associated with the first bedside computing device, and the location information may specify a deployment location of the first bedside computing device in a healthcare facility. For example, the first device profile may indicate that the first bedside computing device is to be used at a particular location within a patient care environment, such as a particular clinic room, a particular operating room, on a particular floor of a hospital, and/or the like. In some instances, this location information may be used by one or more computing devices, such as an EMR server, when logging where particular patient data was captured. Additionally or alternatively, this location information may restrict the locations where the first bedside computing device (e.g., bedside computing device 222) can be used. For example, if the bedside computing device is moved beyond and/or taken outside of the one or more permitted use locations defined in the first device profile, the bedside computing device may present an error message and/or otherwise indicate that it must be returned to one of the permitted use locations specified in the device profile.

In some embodiments, the first device profile may include service information associated with the first bedside computing device, and the service information may specify at least one healthcare service for which the first device profile has been customized. For example, the device profile may customize the bedside computing device for use with a particular healthcare service by defining one or more service-specific settings for particular sensors that may be connected to the bedside computing device, one or more service-specific user interface settings, and/or one or more other service-specific settings that may be used by the bedside computing device. In some instances, the one or more service-specific settings that may be included in the device profile may also affect what information is loaded and/or presented by the bedside computing device. For example, the bedside computing device (e.g., bedside computing device 222) might only load data associated with the particular healthcare service for which it has been customized. For instance, when presenting a patient selection menu, the bedside computing device thus may present a patient list that includes only active patients associated with the specific healthcare service for which the bedside computing device has been configured in accordance with the device profile. In some instances, such a healthcare service may be defined in terms of a location within a hospital (e.g., a particular floor or ward of the hospital), and such service-specific settings may accordingly be associated with one or more specific locations within a hospital or other patient care environment.

In some embodiments, the first device profile may include network information associated with the first bedside computing device, and the network information may specify one or more connection settings to be used by the first bedside computing device. For example, the device profile that is selected by healthcare data management computing platform 210 for bedside computing device 222 may include network information that specifies one or more wired connection settings, one or more wireless connection settings, one or more VPN settings, one or more network security settings and/or the like.

In some embodiments, the first device profile may include access information associated with the first bedside computing device, and the access information may specify one or more authorized users of the first bedside computing device. For example, the access information included in the device profile may identify one or more authorized users of the bedside computing device, such as one or more authorized nurses, doctors, other healthcare providers, and/or the like. Additionally or alternatively, the access information included in the device profile may prevent non-authorized users from using the bedside computing device and/or may prevent specifically-identified individuals from using the bedside computing device. In some instances, the access information included in the device profile may specify one or more authorized users of the bedside computing device by identifying, as authorized, all healthcare providers that are assigned to and/or otherwise associated with one or more specific floors and/or wards within a hospital, one or more specific clinics, and/or one or more specific healthcare services (e.g., one or more medicine services, one or more surgery services, one or more critical care services, one or more specialty services, etc.).

In some embodiments, the first device profile may include user interface information associated with the first bedside computing device, and the user interface information may specify one or more layout settings to be used by the first bedside computing device in presenting one or more graphical user interfaces. For example, the one or more layout settings that may be specified by the user interface information included in the device profile may define which sensor output fields are presented in one or more user interfaces presented by the bedside computing device. Such sensor output fields may, for instance, include a blood pressure sensor output field, a pulse sensor output field, an oxygen saturation sensor output field, and/or one or more other sensor output fields. Additionally or alternatively, the one or more layout settings that may be specified by the user interface information included in the device profile may define where such sensor output fields are presented in one or more user interfaces presented by the bedside computing device. Additionally or alternatively, the one or more layout settings that may be specified by the user interface information included in the device profile may define one or more custom fields that are specific to one or more particular healthcare services that may, for instance, operate within the patient care environment where the bedside computing device is deployed. Such custom fields may, for instance, include a patient weight field (which may, e.g., be presented for outpatient clinical services), a patient position field (which may, e.g., be presented for a surgery service), a patient observations field (which may, e.g., be presented for a critical care service), and/or one or more other custom fields (which may, e.g., be presented for one or more other types of healthcare services that may operate within the patient care environment).

In some embodiments, the first device profile may include application information associated with the first bedside computing device, and the application information may specify one or more applications that are executable on the first bedside computing device. For example, multiple different sensor monitoring applications may be available for and/or loaded on the first bedside computing device, and the application information included in the first device profile may define and/or include one or more specific applications that are executable for the particular bedside computing device. The one or more different sensor monitoring applications may, for instance, enable different types of interaction with and/or monitoring of different sensors.

Continuing to refer to FIG. 3B, at step 8, healthcare data management computing platform 210 may send the selected device profile to bedside computing device 222. For example, at step 8, healthcare data management computing platform 210 may provide the first device profile (which may, e.g., have been selected by healthcare data management computing platform 210 at step 8) to the first bedside computing device (e.g., bedside computing device 222).

At step 9, bedside computing device 222 may receive the device profile from healthcare data management computing platform 210. At step 10, bedside computing device 222 may apply the device profile received from healthcare data management computing platform 210. In applying the device profile, bedside computing device 222 may, for instance, define and/or modify one or more operating settings and/or other configuration settings that may be used by bedside computing device 222 based on the information and/or other settings included in the device profile received from healthcare data management computing platform 210. In some instances, in applying the device profile, bedside computing device 222 also may locally back up and/or otherwise save any and/or all of the information and/or other settings included in the device profile as the last good configuration for bedside computing device 222 (which may, e.g., be subsequently used by bedside computing device 222 during a future startup phase if, for instance, a connection to healthcare data management computing platform 210 cannot be established). Additionally or alternatively, in applying the device profile in some instances, bedside computing device 222 might only apply settings that do not affect the current usage of bedside computing device 222 and/or may wait to apply other settings that do affect the current usage of bedside computing device 222 until bedside computing device 222 is restarted and/or otherwise initialized.

Referring to FIG. 3C, at step 11, bedside computing device 224 may load initial settings and data. For example, bedside computing device 224 may load default network settings, security settings, data capture and/or processing settings, and/or other settings that may be used by bedside computing device 224 in performing various functions. In some instances, bedside computing device 224 may load such initial settings and data after being powered on and/or otherwise initialized. At step 12, bedside computing device 224 may send a connection request to healthcare data management computing platform 210. Such a connection request may, for instance, be sent as a result of, in accordance with, and/or otherwise based on one or more of the initial settings loaded by bedside computing device 224.

At step 13, healthcare data management computing platform 210 may receive a second connection request from a second bedside computing device. For example, at step 13, healthcare data management computing platform 210 may receive the connection request sent by bedside computing device 224. At step 14, healthcare data management computing platform 210 may identify and authenticate the bedside computing device (e.g., bedside computing device 224). In identifying and authenticating the bedside computing device, healthcare data management computing platform 210 may, for example, request, receive, and/or validate one or more unique device identifiers associated with bedside computing device 224, one or more network addresses associated with bedside computing device 224, location information associated with bedside computing device 224 (which may, e.g., specify a deployment location of the bedside computing device), and/or one or more credentials and/or tokens associated with bedside computing device 224.

At step 15, healthcare data management computing platform 210 may establish a second network connection to a second bedside computing device different from the first bedside computing device. For example, after identifying and/or authenticating bedside computing device 224 (e.g., at step 14), healthcare data management computing platform 210 may establish a network connection with bedside computing device 224 (e.g., at step 15).

Referring to FIG. 3D, at step 16, healthcare data management computing platform 210 may select a second device profile for the second bedside computing device. For example, at step 16, healthcare data management computing platform 210 may select a device profile for bedside computing device 224. Such a device profile may, for instance, be selected by healthcare data management computing platform 210 for bedside computing device 224 from a set of predefined device profiles based on one or more factors, similar to how such a device profile was selected for bedside computing device 222. The second device profile (which may, e.g., be selected by healthcare data management computing platform 210 for bedside computing device 224) may be similar to the first device profile (which may, e.g., be selected by healthcare data management computing platform 210 for bedside computing device 222). For example, healthcare data management computing platform 210 may select a device profile for the second bedside computing device (e.g., bedside computing device 224) based on one or more properties associated with the second bedside computing device. In addition, the second device profile may include one or more configuration settings for the second bedside computing device, and/or may include other information that may be used by the second bedside computing device. For example, the second device profile may include location information, service information, network information, access information, user interface information, and/or application information for the second bedside computing device, similar to how the first device profile may include such information for the first bedside computing device. The second device profile may be different from the first device profile in that the second device profile may include location information, service information, network information, access information, user interface information, and/or application information that is specific to the second bedside computing device, whereas the first device profile may include location information, service information, network information, access information, user interface information, and/or application information that is specific to the first bedside computing device (which may, e.g., be different from the location information, service information, network information, access information, user interface information, and/or application information of the second bedside computing device because of one or more differences between the first bedside computing device and the second bedside computing device).

At step 17, healthcare data management computing platform 210 may send the selected device profile to bedside computing device 224. For example, at step 17, healthcare data management computing platform 210 may provide the second device profile (which may, e.g., have been selected by healthcare data management computing platform 210 at step 16) to the second bedside computing device (e.g., bedside computing device 224).

At step 18, bedside computing device 224 may receive the device profile from healthcare data management computing platform 210. At step 19, bedside computing device 224 may apply the device profile received from healthcare data management computing platform 210. In applying the device profile, bedside computing device 224 may, for instance, define and/or modify one or more operating settings and/or other configuration settings that may be used by bedside computing device 224 based on the information and/or other settings included in the device profile received from healthcare data management computing platform 210.

Subsequently, healthcare data management computing platform 210 may continue to receive one or more connection requests from various other bedside computing devices, and healthcare data management computing platform 210 may provision such bedside computing devices with one or more device profiles as such requests are received (e.g., by performing and/or repeating one or more steps of the example event sequence illustrated in FIGS. 3A-3D with respect to such other bedside computing devices). Having described an example in which healthcare data management computing platform 210 may configure a number of different bedside computing devices with device profiles, an example illustrating how a particular bedside computing device may capture and process sensor data (e.g., after it has been provisioned with a device profile by healthcare data management computing platform 210) will now be discussed.

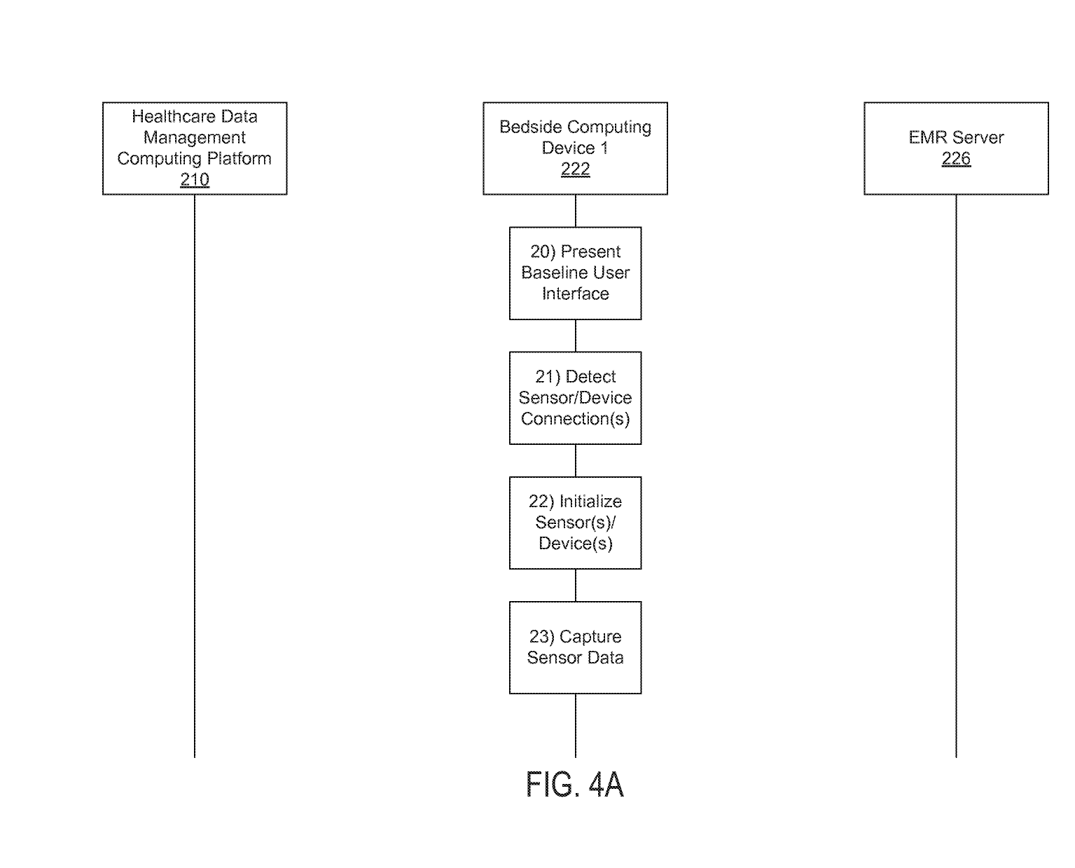

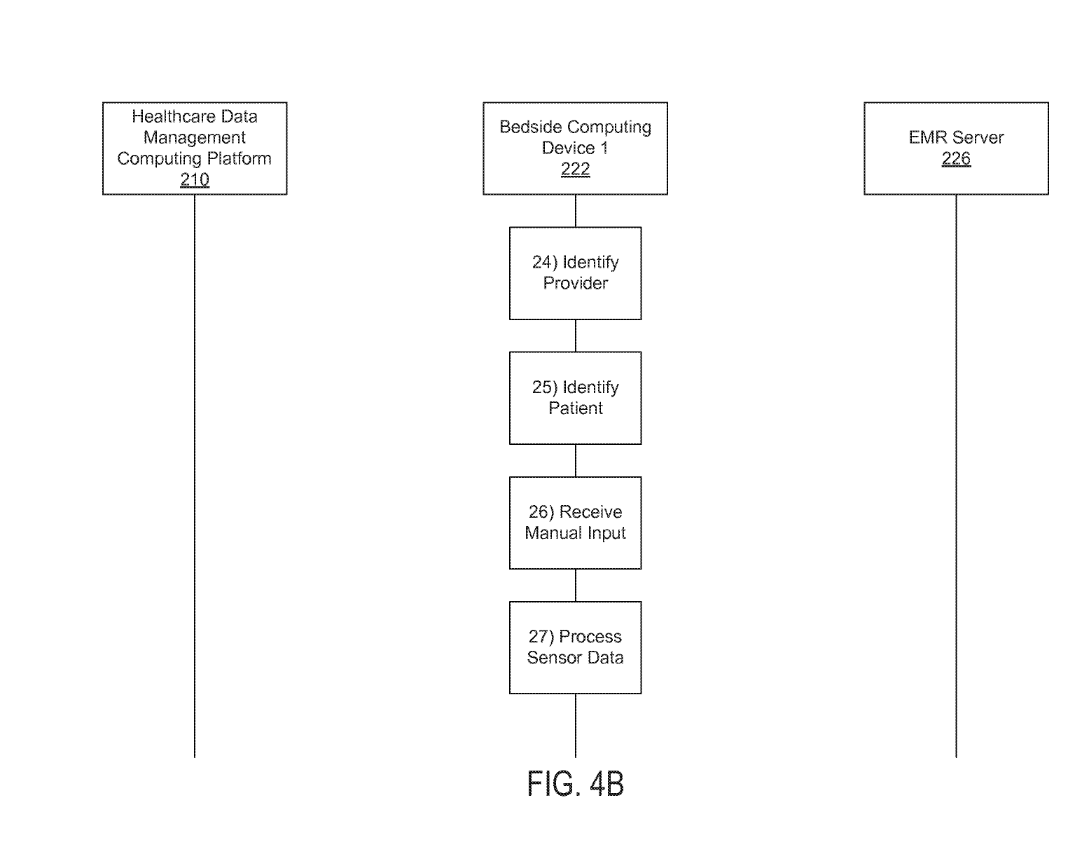

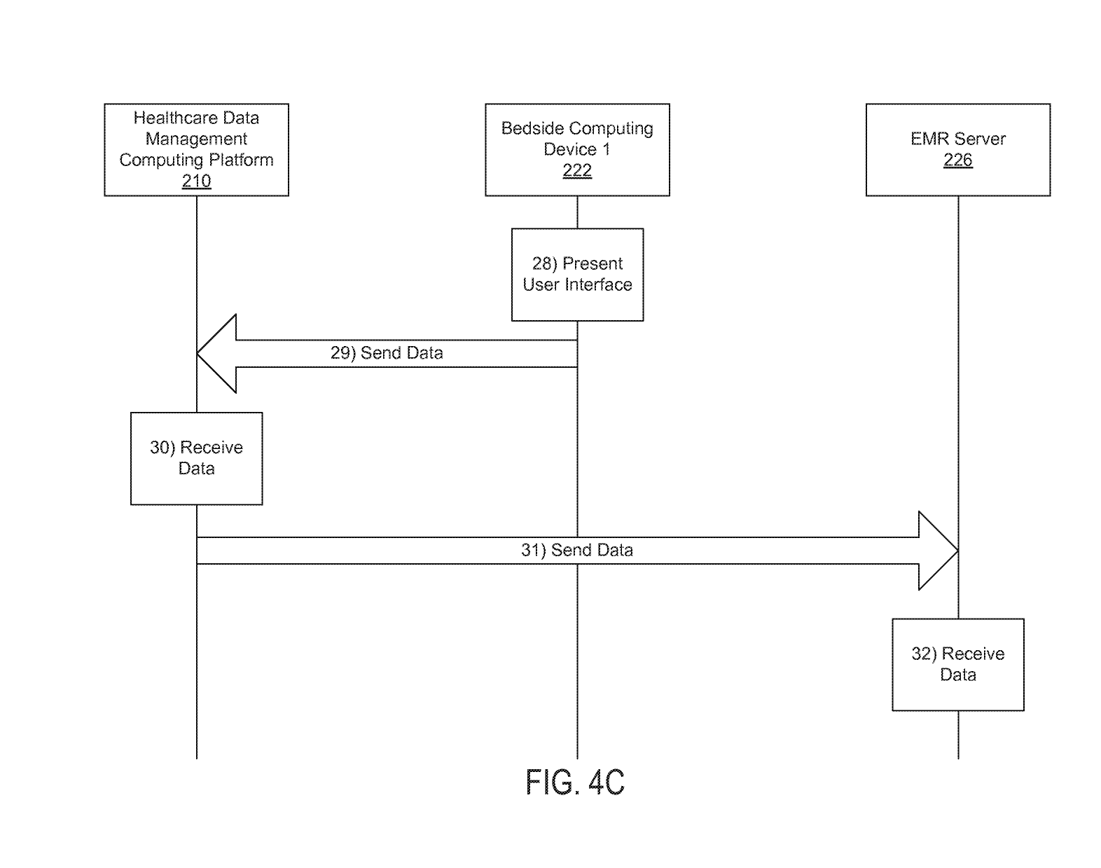

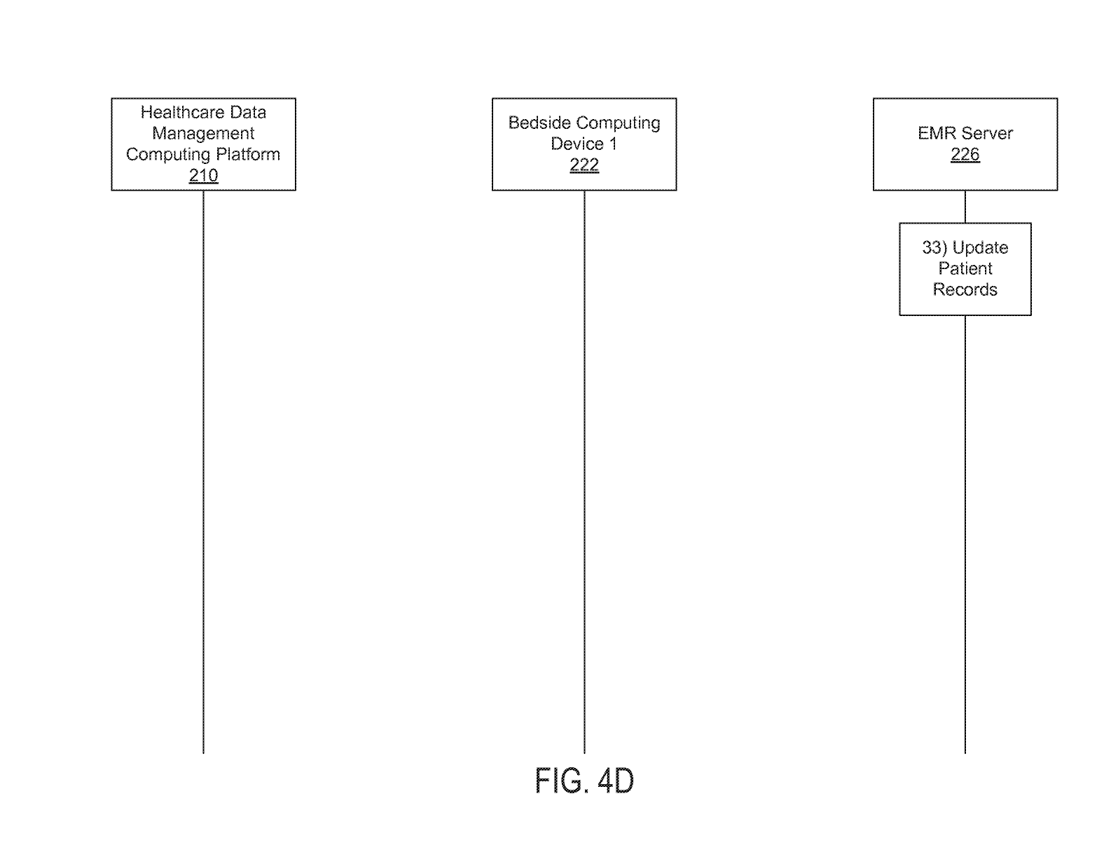

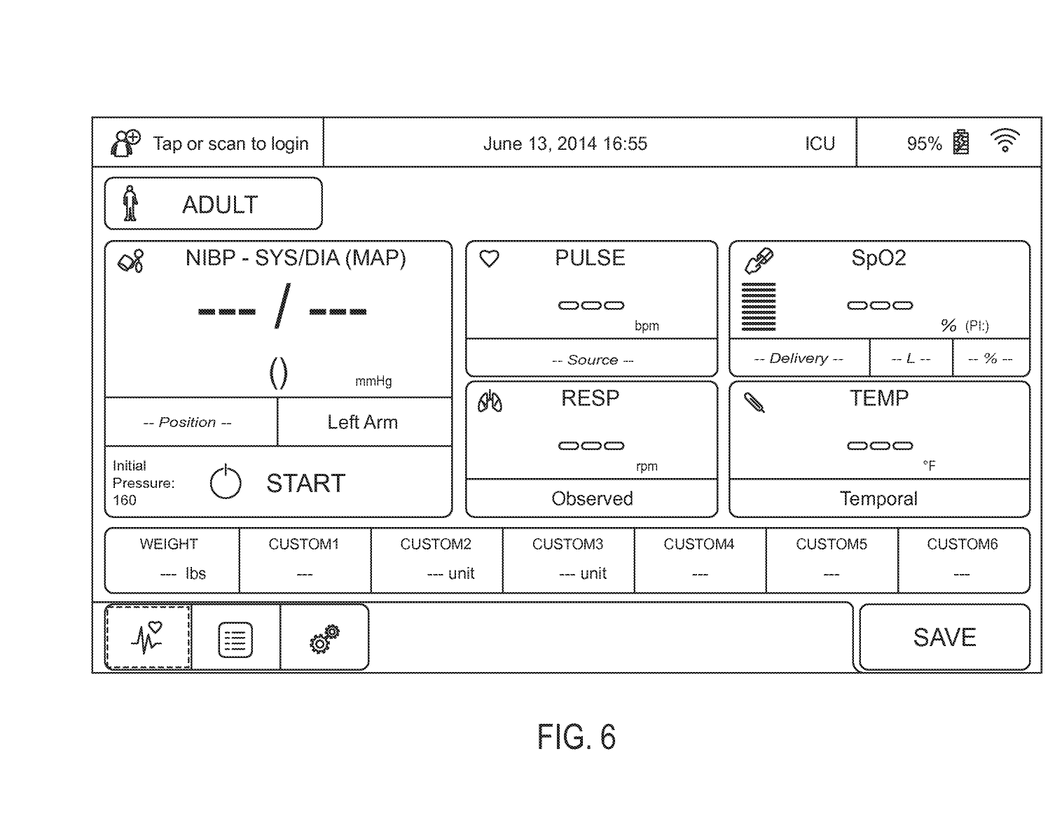

FIGS. 4A-4D depict an example of an event sequence for capturing and processing sensor data in accordance with one or more illustrative aspects of the disclosure. Referring to FIG. 4A, at step 20, bedside computing device 222 may present a baseline user interface. For example, at step 20, bedside computing device 222 may generate, display, and/or otherwise present a graphical user interface similar to the example user interface depicted in FIG. 6. As seen in FIG. 6, such a user interface may be configured to include status information associated with bedside computing device 222, a number of sensor output fields, a number of customizable fields, and/or other information associated with bedside computing device 222 and/or one or more sensors and/or other devices that may be connected to bedside computing device 222.

Referring again to FIG. 4A, at step 21, bedside computing device 222 may detect one or more sensor connections and/or one or more medical device connections. For example, at step 21, bedside computing device 222 may detect that one or more sensors and/or medical devices have been connected to one or more data ports of bedside computing device 222 (e.g., based on signals received via such ports) and/or based on receiving one or more requests from such sensors and/or medical devices to initiate one or more corresponding wired and/or wireless connections with such sensors and/or medical devices.

At step 22, bedside computing device 222 may initialize the one or more detected sensors and/or the one or more detected medical devices. In initializing the one or more detected sensors and/or medical devices, bedside computing device 222 may, for instance, identify the connected sensors and/or devices, load one or more applicable device drivers, establish one or more wired and/or wireless connections to facilitate communication with the connected sensors and/or devices, and/or perform any other actions that might be needed to begin receiving and/or processing data from the detected sensors and/or devices.

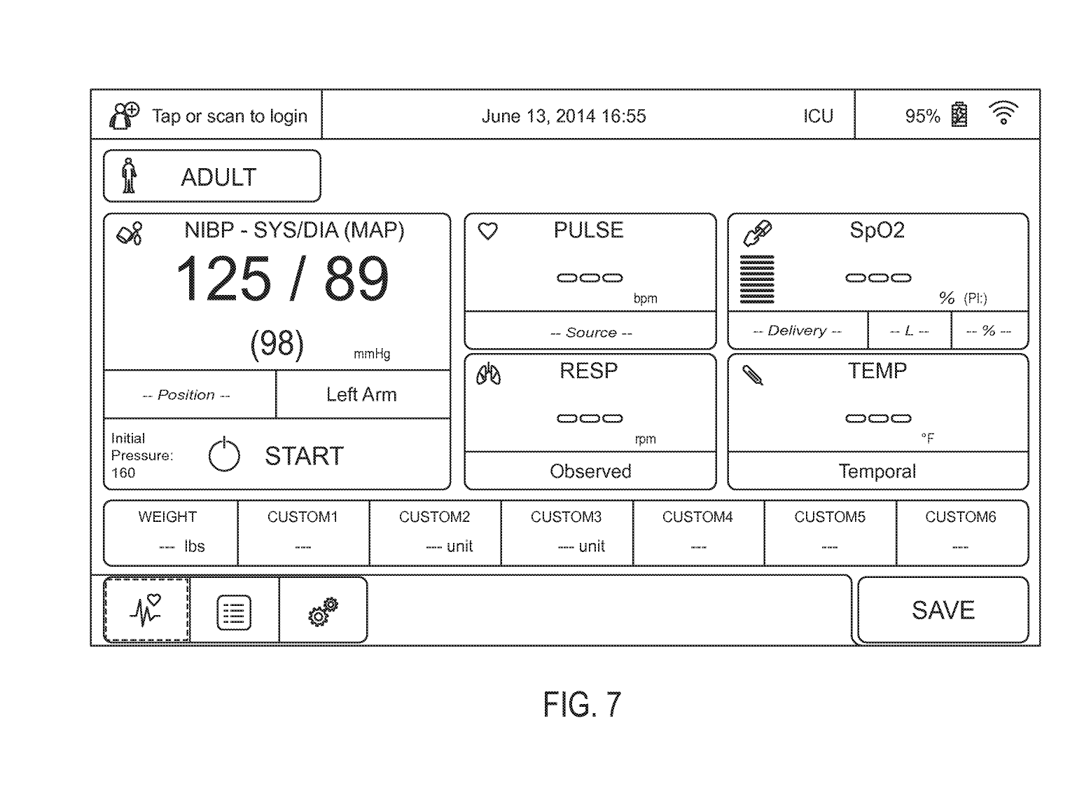

At step 23, bedside computing device 222 may capture sensor data from the one or more sensors and/or devices (which may, e.g., have been detected at step 21 and/or initialized at step 22). For example, at step 23, bedside computing device 222 may capture sensor data received from one or more sensors connected to bedside computing device 222. As bedside computing device 222 begin capturing sensor data from such sensors and/or devices, bedside computing device 222 may update the previously-presented user interface and/or otherwise present a user interface that includes information associated with the sensor data being captured by bedside computing device 222. For example, at step 23, bedside computing device 222 may generate, display, and/or otherwise present a graphical user interface similar to the example user interface depicted in FIG. 7. As seen in FIG. 7, such a user interface may be configured to include information associated with the sensor data being captured by bedside computing device 222, such as blood pressure information captured from a blood pressure sensor, as shown in the illustrated example.

In some embodiments, at least a portion of the sensor data may be received from at least one sensor that is directly connected to the bedside computing device. For example, in some instances, at least a portion of the sensor data that is captured by bedside computing device 222 (e.g., at step 23) may be received from one or more sensors that are directly connected to bedside computing device 222. As discussed above, one or more sensors may be directly connected to bedside computing device 222 (e.g., with or without the use of a module or other port adapter, via a wired connection and/or a wireless connection, etc.) and may provide raw signal data (e.g., analog signal data) to bedside computing device 222. In addition, bedside computing device 222 may, for instance, process such raw signal data to determine one or more values associated with one or more conditions being monitored by the one or more directly connected sensors. For example, a blood pressure sensor may provide raw analog signal data to the bedside computing device when it is directly connected to the bedside computing device, and the bedside computing device may determine a blood pressure value based on the raw analog signal data received from the blood pressure sensor. The determined blood pressure value may then be displayed and/or otherwise presented in the user interface by the bedside computing device, as illustrated in FIG. 7.

In other embodiments, at least a portion of the sensor data may be received from at least one sensor that is connected to the bedside computing device via at least one medical device. For example, in some instances, at least a portion of the sensor data that is captured by bedside computing device 222 (e.g., at step 23) may be received from one or more sensors that are connected to bedside computing device 222 via one or more medical devices. As discussed above, such a medical device may, for instance, receive and/or process raw signal data from one or more sensors (e.g., to determine one or more values associated with one or more conditions being monitored by the one or more sensors) and subsequently may provide the processed data (e.g., as digital data) to the bedside computing device. The bedside computing device may then present and/or otherwise use the processed data, but might not need to further process and/or analyze the data received from the medical device, since the values associated with the monitored conditions may have been already determined by the medical device. For example, a pulse oximeter sensor may be indirectly connected to the bedside computing device via a medical device, and the medical device may receive raw analog signal data from the pulse oximeter sensor. Subsequently, the medical device may process the raw analog signal data to determine values for the pulse rate and the oxygen saturation being monitored by the sensor, and the medical device may provide digital information to the bedside computing device that includes the determined pulse rate and oxygen saturation values. The pulse rate and oxygen saturation values that are received by the bedside computing device from the medical device may then be displayed and/or otherwise presented in the user interface.

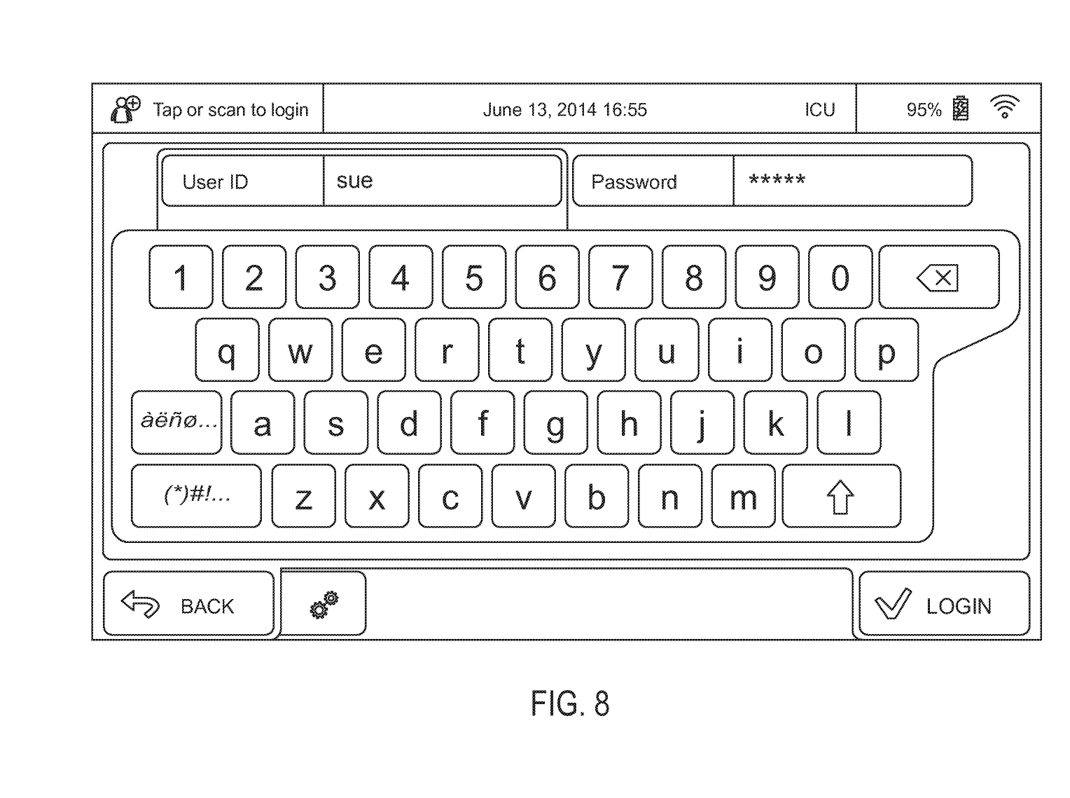

Referring to FIG. 4B, at step 24, bedside computing device 222 may identify and/or authenticate a healthcare provider, such as a nurse or doctor, who may be using bedside computing device 222. In identifying and/or authenticating a healthcare provider, bedside computing device 222 may generate, display, and/or otherwise present a graphical user interface similar to the example user interface depicted in FIG. 8. As seen in FIG. 8, such a user interface may be configured to prompt a user of bedside computing device 222 to enter a username, password, and/or other login credentials (which may, e.g., be subsequently validated by bedside computing device 222 to confirm that the user of bedside computing device 222 is authorized to access bedside computing device 222). In some instances, a user of bedside computing device 222 may authenticate with bedside computing device 222 using a barcode reader (which may, e.g., be used to read a username from a barcode printed on the user's hospital identification card) that may be included in and/or connected to bedside computing device 222. Additionally or alternatively, a user of bedside computing device 222 may authenticate with bedside computing device 222 using a radio-frequency identification (RFID) tag reader (which may, e.g., be used to read an RFID tag associated with the user of bedside computing device 222, such as an RFID tag that may be embedded in the user's hospital identification card) that may be included in and/or connected to bedside computing device 222. In addition, although the example illustrated here involves the healthcare provider being identified by bedside computing device 222 after one or more sensors are connected to bedside computing device 222 and bedside computing device 222 begins capturing sensor data, these steps could occur in any order. For example, in some instances, a healthcare provider who is using bedside computing device 222 may be identified and/or authenticated before one or more sensors are connected to bedside computing device 222 and/or before sensor data is captured by bedside computing device 222.

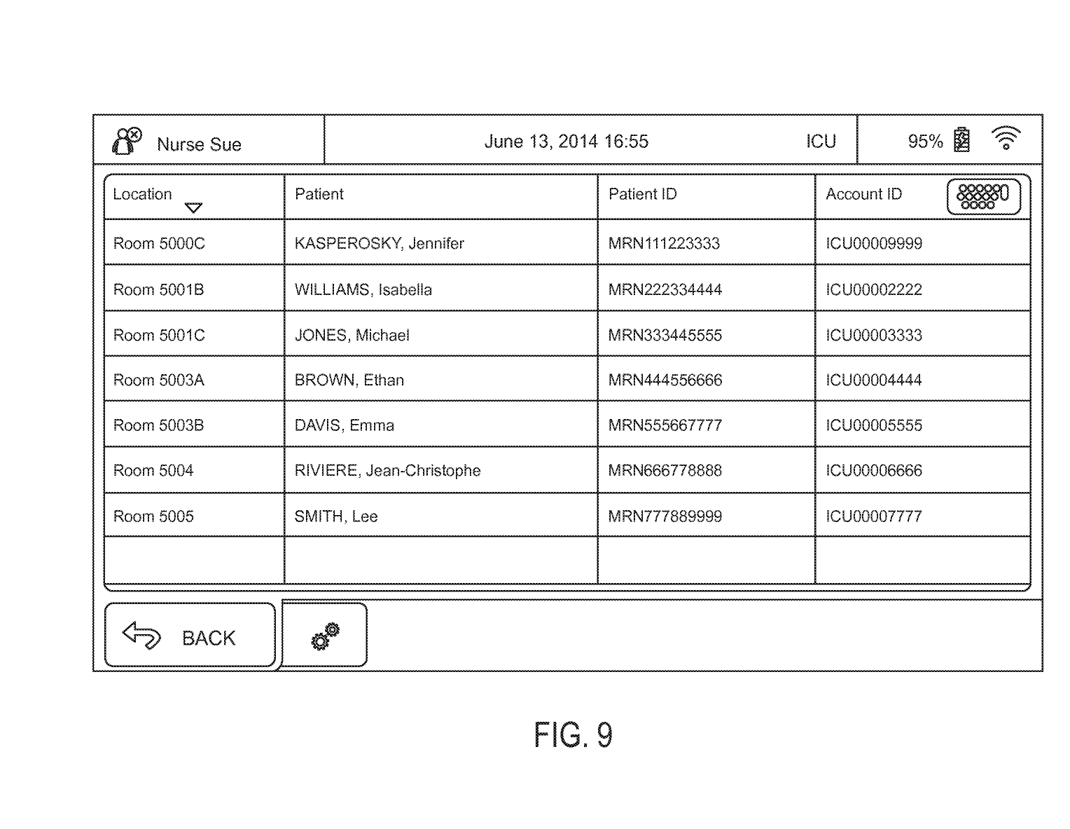

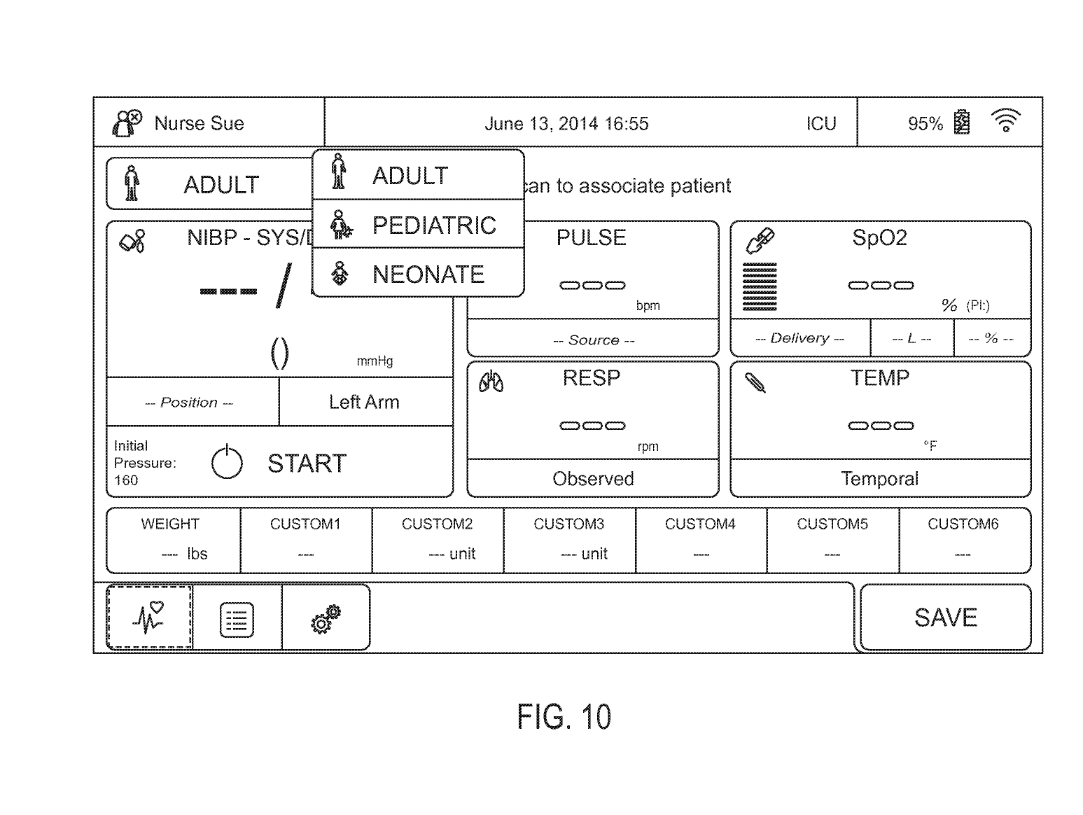

Referring again to FIG. 4B, at step 25, bedside computing device 222 may identify and/or authenticate a patient, such as the patient being monitored by the one or more sensors connected to bedside computing device 222. In identifying and/or authenticating the patient, bedside computing device 222 may generate, display, and/or otherwise present a graphical user interface similar to the example user interface depicted in FIG. 9. As seen in FIG. 9, such a user interface may be configured to prompt a user of bedside computing device 222 to select the patient being monitored from a patient list that may be presented by bedside computing device 222. As discussed above, such a patient list may be selected and/or presented by bedside computing device 222 based on a device profile (which may, e.g., include service information and/or location information that may be used in determining such a patient list) that has been selected for and/or applied to bedside computing device 222. Additionally or alternatively, in identifying and/or authenticating the patient, bedside computing device 222 may generate, display, and/or otherwise present a graphical user interface similar to the example user interface depicted in FIG. 10. As seen in FIG. 10, such a user interface may be configured to prompt a user of bedside computing device 222 (who may, e.g., be the healthcare provider identified at step 24) to enter information about the type of patient being monitored (e.g., adult, pediatric, neonate, etc.). In some instances, information associated with the type of patient being monitored may be determined automatically by bedside computing device 222 (e.g., based on one or more predefined age thresholds to differentiate between adult patients, pediatric patients, and/or neonate patients and/or based on information that may be accessed and/or loaded by bedside computing device 222 after the patient is selected from the patient list shown in FIG. 9). Additionally or alternatively, information associated with the type of patient being monitored may be provided to bedside computing device 222 by one or more other systems, such as an Admit/Discharge/Transfer (ADT) system that may be operated by and/or otherwise associated with the healthcare organization that may operate healthcare data management computing platform 210 and/or bedside computing device 222. In some instances, rather than selecting the patient being monitored by bedside computing device 222 from a patient list, such as the patient list shown in FIG. 9, a user of bedside computing device 222 may identify and/or authenticate the patient by entering a unique identifier associated with the patient and/or by scanning a barcode associated with the patient, such as a barcode that may be printed on the patient's hospital wristband.

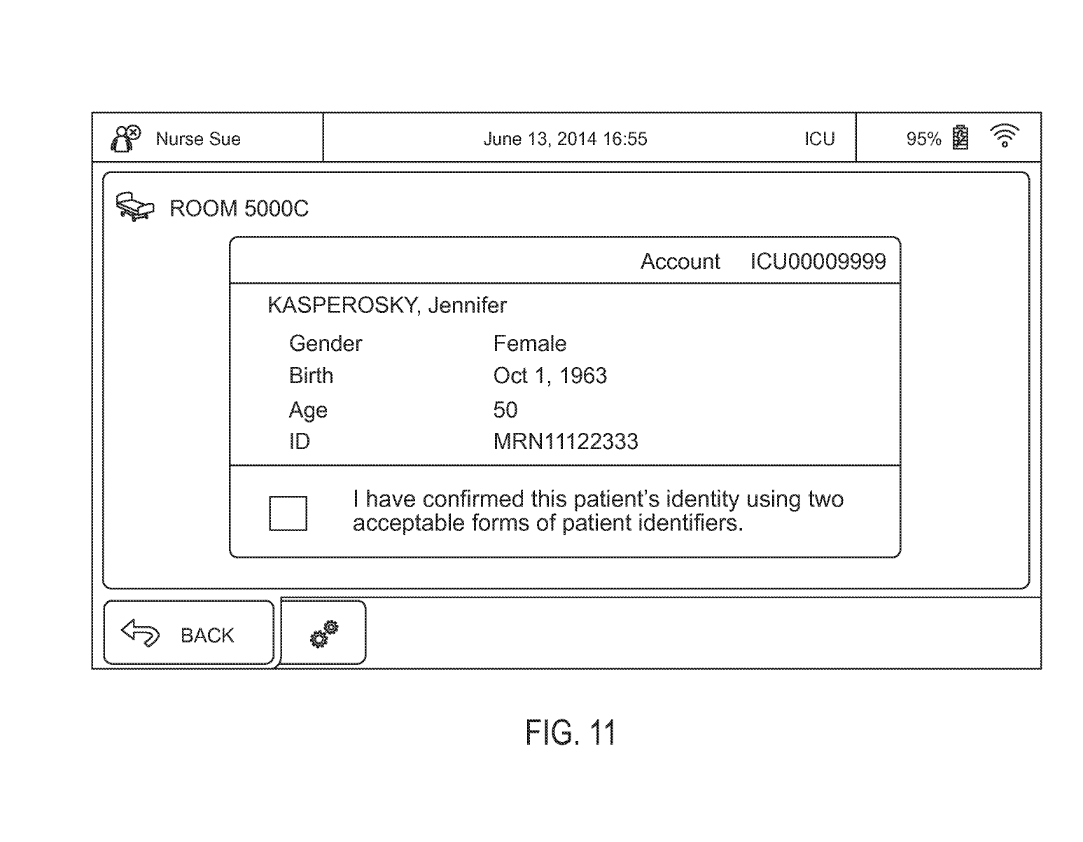

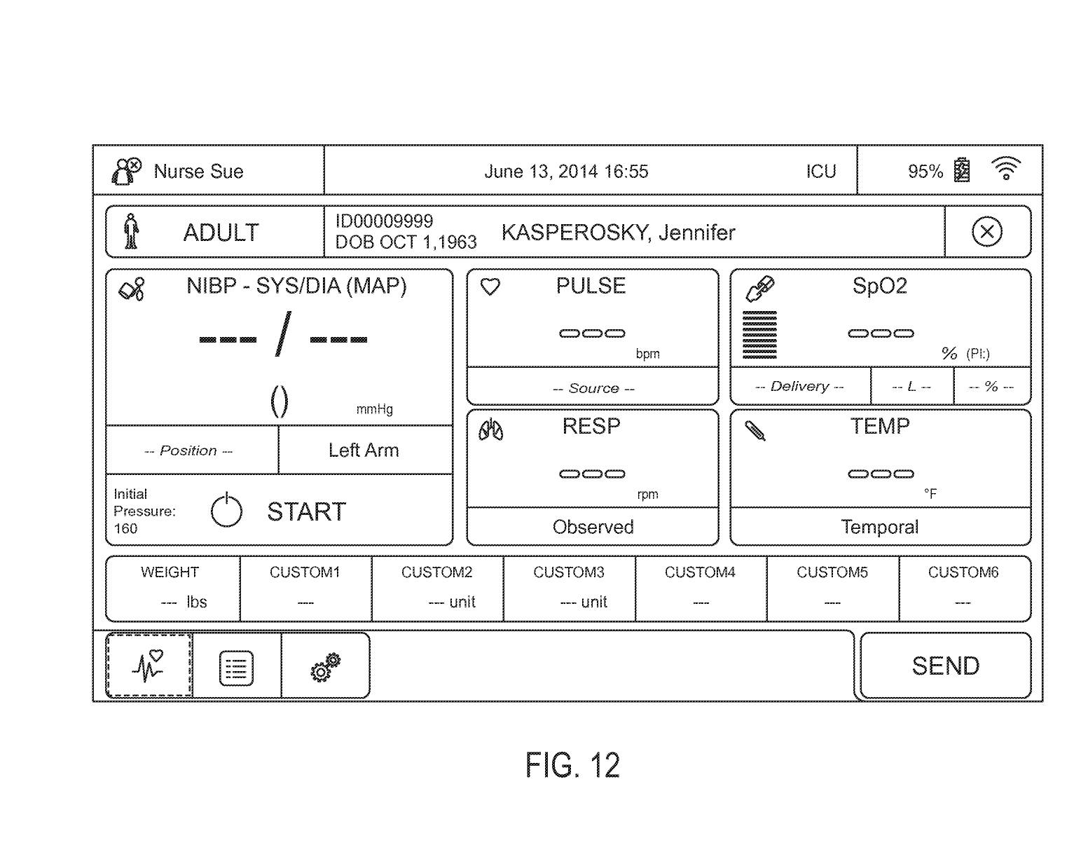

Additionally or alternatively, in identifying and/or authenticating the patient, bedside computing device 222 may generate, display, and/or otherwise present a graphical user interface similar to the example user interface depicted in FIG. 11. As seen in FIG. 11, such a user interface may be configured to prompt the user of bedside computing device 222 (who may, e.g., be the healthcare provider identified at step 24) to confirm the patient's identity by verifying one or more (and in typical instances, at least two) pieces of identifying information associated with the patient, such as their name, date of birth, age, and/or unique identifier (which may, e.g., be scanned from the patient's hospital bracelet). Additionally or alternatively, in identifying and/or authenticating the patient, bedside computing device 222 may generate, display, and/or otherwise present a graphical user interface similar to the example user interface depicted in FIG. 12. As seen in FIG. 12, after the patient has been identified and/or his or her identity has been confirmed, bedside computing device 222 may again present a user interface that is configured to include information associated with the sensor data being captured by bedside computing device 222, along with information identifying the patient that is being monitored by bedside computing device 222. In addition, while the example illustrated here involves the patient being identified by bedside computing device 222 after one or more sensors are connected to bedside computing device 222 and after the healthcare provider using bedside computing device 222 has been identified, these steps could occur in any order. For example, in some instances, the patient who is being monitored by bedside computing device 222 may be identified and/or authenticated before any sensors are connected to bedside computing device 222 and/or before the healthcare provider using bedside computing device 222 has been identified.

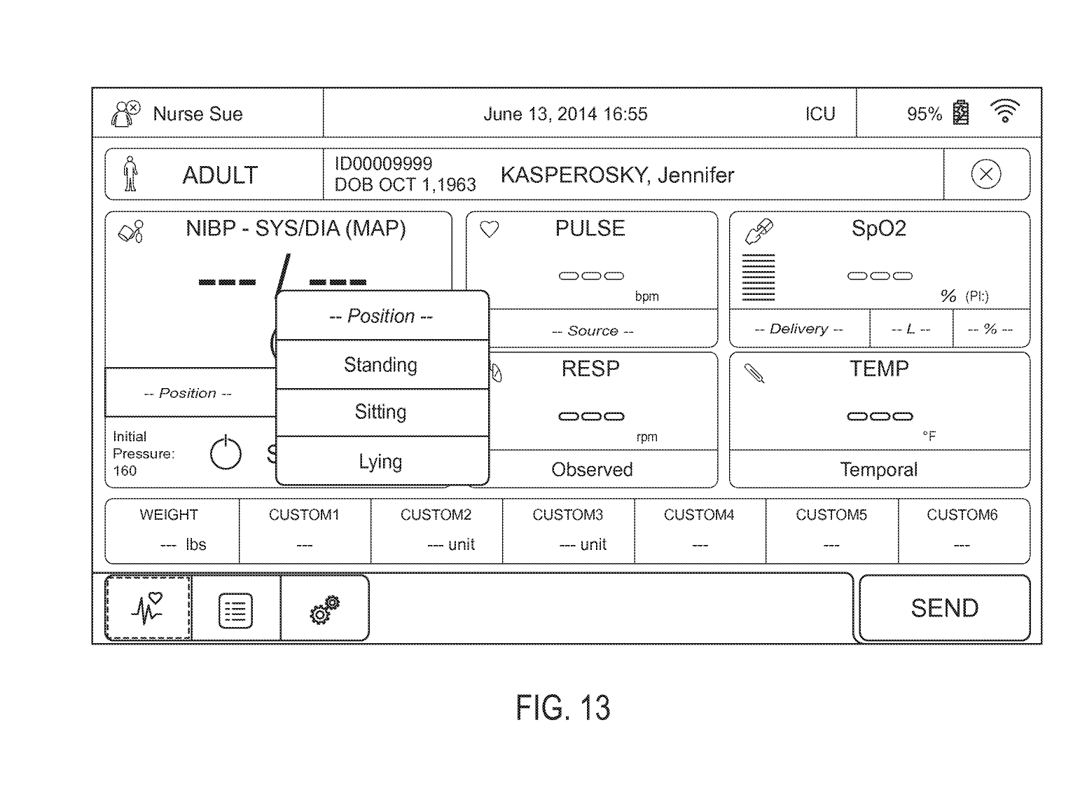

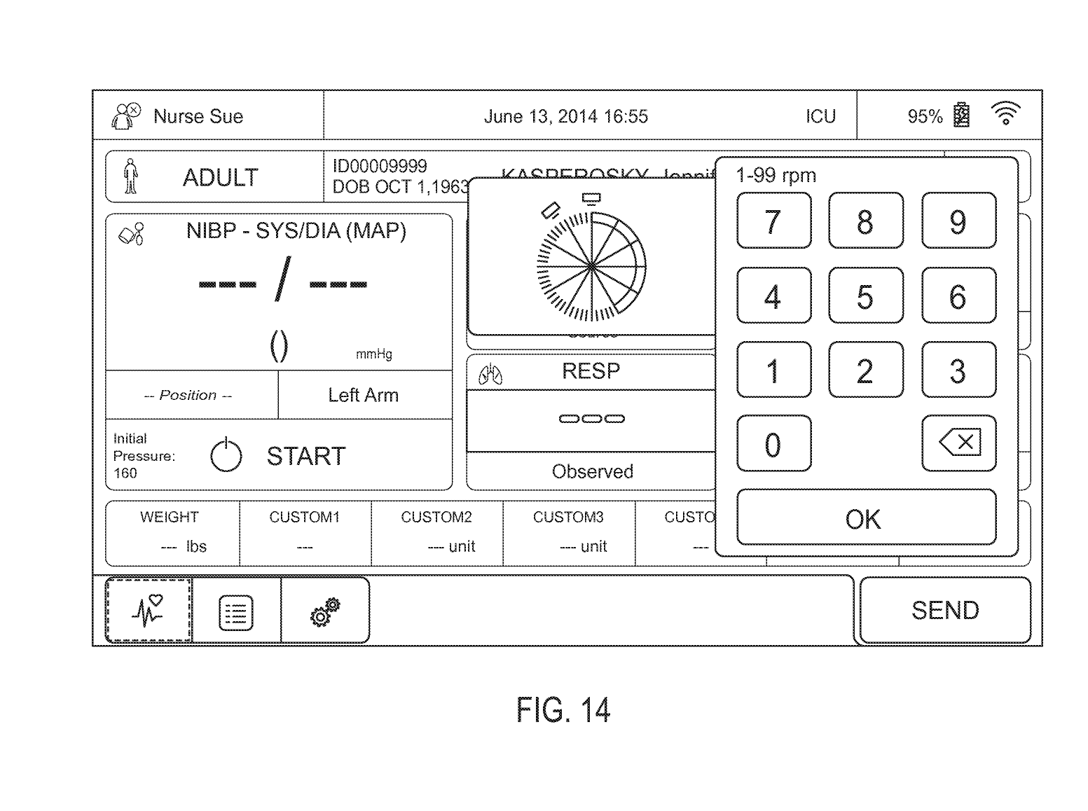

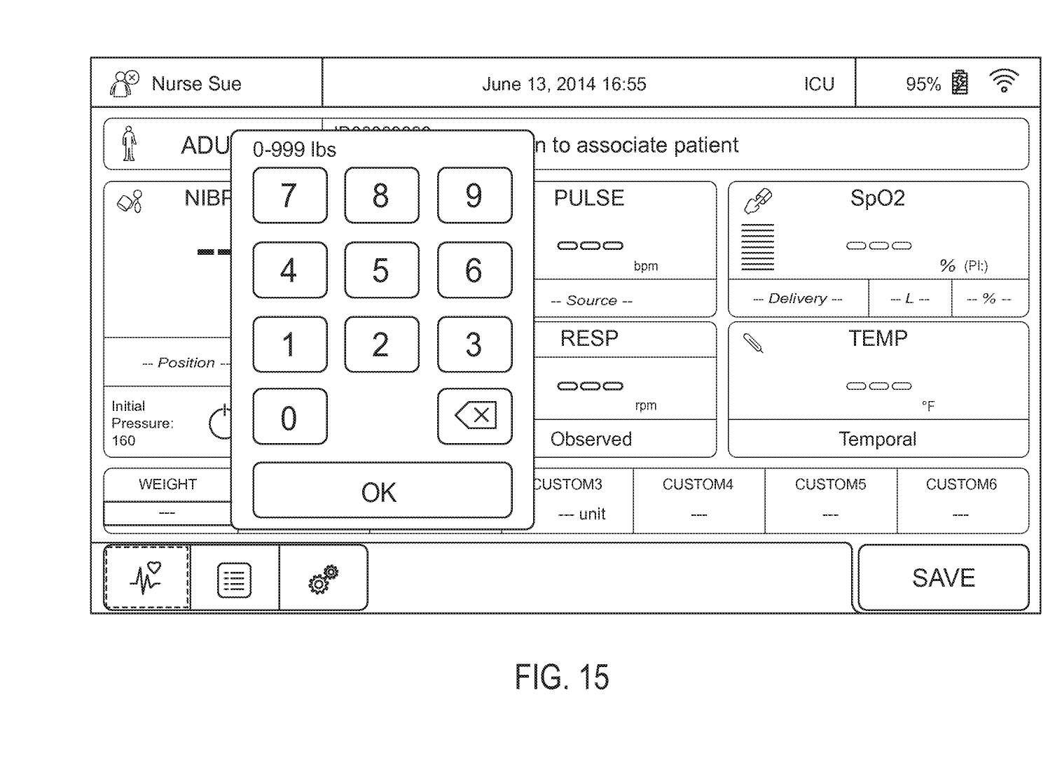

Referring again to FIG. 4B, at step 26, bedside computing device 222 may receive manual input from a user of bedside computing device 222 (who may, e.g., be the healthcare provider identified at step 24). Such manual input may, for example, include information about the patient's physical position, the patient's weight, and/or the healthcare provider's observations of the patient. Additionally or alternatively, any and/or all of the manual input may be used by bedside computing device 222 in presenting one or more user interfaces and/or in updating one or more custom fields that may be included in such user interfaces. For example, in receiving manual input, bedside computing device 222 may generate, display, and/or otherwise present a graphical user interface similar to the example user interface depicted in FIG. 13. As seen in FIG. 13, such a user interface may be configured to prompt a user of bedside computing device 222 to identify the patient's physical position (e.g., standing, sitting, lying, etc.). Additionally or alternatively, in receiving manual input, bedside computing device 222 may generate, display, and/or otherwise present a graphical user interface similar to the example user interface depicted in FIG. 14. As seen in FIG. 14, such a user interface may be configured to prompt a user of bedside computing device 222 to enter information associated with the healthcare provider's observations of the patient, such as the patient's observed respiratory rate. Additionally or alternatively, in receiving manual input, bedside computing device 222 may generate, display, and/or otherwise present a graphical user interface similar to the example user interface depicted in FIG. 15. As seen in FIG. 15, such a user interface may be configured to prompt a user of bedside computing device 222 to enter the patient's weight, which may, for instance, be measured by the healthcare provider operating bedside computing device 222. In addition, while the example illustrated here involves manual input being entered into bedside computing device 222 after one or more sensors are connected to bedside computing device 222, after the healthcare provider using bedside computing device 222 has been identified, and after the patient has been identified, these steps could occur in any order. For example, in some instances, such manual input may be received by bedside computing device 222 before any and/or all of these other events occur. Additionally, in receiving manual input in some instances, bedside computing device 222 may receive input from a user of bedside computing device 222 that overrides one or more data capture fields that may be populated with data captured by and/or otherwise associated with a sensor and/or medical device that is connected to bedside computing device 222. For example, in some instances, bedside computing device 222 may receive manual input that overrides a blood pressure value field that might otherwise be populated with data captured by a blood pressure sensor that is connected to bedside computing device 222. Additionally or alternatively, the device profile being used by bedside computing device 222 may define valid ranges for each field (which may, e.g., be associated with a particular patient vital sign being monitored by bedside computing device 222), and bedside computing device 222 may prevent a user from manually entering a value that is outside of the valid range corresponding to the particular field. For example, the device profile being used by bedside computing device 222 may define a valid range for a patient temperature field, and bedside computing device 222 may prevent a user from manually entering a temperature value for the patient that is outside of the valid range for the patient temperature field defined in the device profile.

Referring again to FIG. 4B, at step 27, bedside computing device 222 may process the captured sensor data. For example, at step 27, bedside computing device 222 may process the sensor data to determine patient vital signs information. In processing the sensor data to determine patient vital signs information, bedside computing device 222 may, for instance, analyze, evaluate, and/or otherwise process the captured sensor data to determine one or more values for the patient's blood pressure, pulse rate, oxygen saturation, respiratory rate, temperature, and/or any other vital signs that may be determined based on the sensor data captured by bedside computing device 222. Additionally or alternatively, such vital signs may be tracked over and/or determined as a function of time based on the sensor data that may be captured by bedside computing device 222 over a course of time (e.g., the time from when the one or more sensors were connected to the patient by the healthcare provider to the current time). In some instances, bedside computing device 222 may synchronize its system time with the system time used and/or maintained by healthcare data management computing platform 210.

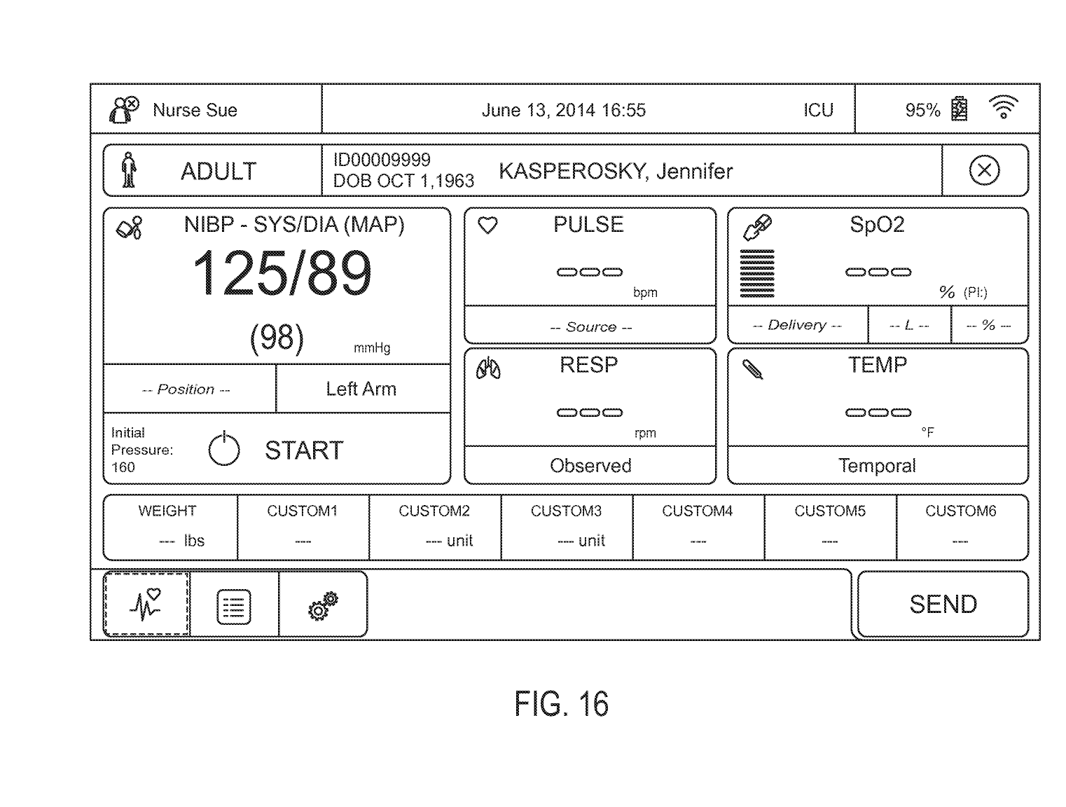

Referring to FIG. 4C, at step 28, bedside computing device 222 may present one or more user interfaces that include any and/or all of the processed sensor data. For example, at step 28, bedside computing device 222 may present at least one graphical user interface that includes at least a portion of the patient vital signs information. In presenting one or more user interfaces that include patient vital signs information, bedside computing device 222 may generate, display, and/or otherwise present a graphical user interface similar to the example user interface depicted in FIG. 16. As seen in FIG. 16, such a user interface may present a single screen from which the healthcare provider may acquire, enter, review, and/or transmit validated vital signs information and/or patient context information, including information associated with the patient's blood pressure, oxygen saturation, pulse rate, temperature, and/or one or more custom vitals (e.g., up to seven custom vitals in the example illustrated in FIG. 16). In accordance with one or more aspects of the disclosure, by presenting such a user interface, bedside computing device 222 may provide a number of advantages over existing and/or conventional vital signs monitors. For example, bedside computing device 222 may present many different types of vital signs information, including custom vital signs information, in a single user interface. In addition, bedside computing device 222 may enable a user of bedside computing device 222, such as a healthcare provider who may be caring for the patient being monitored by bedside computing device 222, to perform a number of different functions from the same single user interface, including acquisition of vital signs information, monitoring of vital signs information, and reporting and/or transmitting of vital signs information to an EMR server and/or other computer systems, as discussed in greater detail below.

Subsequently, bedside computing device 222 may continue capturing and/or processing sensor data, as well as updating the user interface to include current vital signs information based on the captured and/or processed data, until one or more sensors are disconnected and/or the process is stopped by the user, for instance. Such continued monitoring and/or processing may be handled as a service by the software architecture implemented by bedside computing device 222.

Referring again to FIG. 4C, at step 29, bedside computing device 222 may send the processed sensor data to healthcare data management computing platform 210. For example, at step 29, bedside computing device 222 may provide the patient vital signs information to a server computing device that is configured to receive vital signs information from one or more additional bedside computing devices different from the first bedside computing device. For instance, as discussed in greater detail below, healthcare data management computing platform 210 may be configured to receive vital signs information from bedside computing device 222 as well as one or more other bedside computing devices that may be deployed in the patient care environment, such as bedside computing device 224. In addition, and as discussed in greater detail below, bedside computing device 222 may send the vital signs information to healthcare data management computing platform 210, which may then send the vital signs information to an EMR server. Both bedside computing device 222 and healthcare data management computing platform 210 may cache the vital signs information when sending the vital signs information (e.g., as each transmission may represent a potential point of failure), and healthcare data management computing platform 210 may receive, further process, and/or transmit and/or otherwise send the vital signs information to the EMR server. In one or more arrangements, both the bedside computing device 222 and the healthcare data management computing platform 210 may be configured to filter and/or otherwise process the vital signs information before sending the vital signs information. In addition, in one or more embodiments, the one or more bedside computing devices (e.g., bedside computing device 222 and bedside computing device 224) and/or the one or more computing platforms and/or other servers that may be included in computing environment 200 (e.g., healthcare data management computing platform 210) may be configured to encrypt any and/or all data prior to sending data to another computing device or system. For example, in sending the processed sensor data to healthcare data management computing platform 210, bedside computing device 222 may first encrypt the processed sensor data prior to sending the data to healthcare data management computing platform 210, and upon receiving the data, healthcare data management computing platform 210 may decrypt the data received from bedside computing device 222 (e.g., prior to sending vital signs information on to an EMR server and/or otherwise using the processed sensor data).

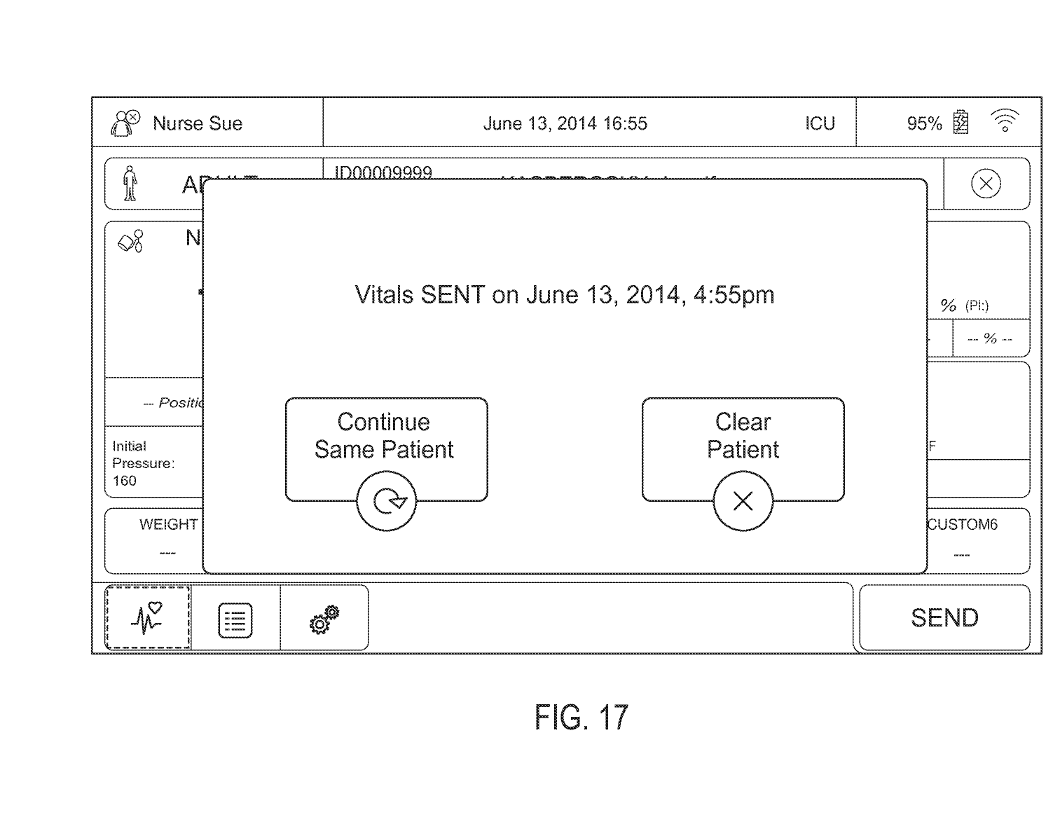

In some instances, in sending the processed sensor data to healthcare data management computing platform 210, bedside computing device 222 may also send the raw sensor data captured from one or more sensors directly connected to bedside computing device 222. In sending the processed sensor data to healthcare data management computing platform 210, bedside computing device 222 may generate, display, and/or otherwise present a graphical user interface similar to the example user interface depicted in FIG. 17. As seen in FIG. 17, such a user interface may be configured to prompt a user of bedside computing device 222 to send the processed sensor data (which may, e.g., include the patient's vital signs information) to a central server (e.g., healthcare data management computing platform 210), notify the user when such data has been sent to the server, and/or provide the user with one or more options, such as an option to continue monitoring the same patient and/or an option to begin monitoring a different patient.

In some embodiments, the server computing device may be further configured to provide at least a portion of the patient vital signs information to at least one electronic medical records (EMR) system. For example, and as discussed in greater detail below, healthcare data management computing platform 210 may be configured to provide at least a portion of the patient vital signs information to at least one EMR system, such as EMR server 226. In some instances, healthcare data management computing platform 210 may additionally or alternatively provide at least a portion of the patient vital signs information to one or more other systems (which may, e.g., be operated by and/or otherwise used by a healthcare organization operating healthcare data management computing platform 210), such as one or more alarm notification systems, one or more patient surveillance systems, one or more clinical research systems, and/or one or more other clinical information systems. In some instances, healthcare data management computing platform 210 may adapt the patient vital signs information and/or other data that may be provided to one or more other systems to suit the particular requirements of the recipient system. For example, healthcare data management computing platform 210 may filter, format, and/or otherwise convert data differently for each of the one or more recipient systems so as to provide a “multi-feed” output feature in which healthcare data management computing platform 210 provides multiple processing channels with each of the different channels providing output to one or more different systems. In some instances, such multi-feed output may be provided by a data management module (DMM) that may be included in and/or otherwise provided by healthcare data management computing platform 210.

In some embodiments, the bedside computing device may be configured to capture and process the sensor data based on a device profile for the bedside computing device that is managed, at least in part, by the server computing device. For example, bedside computing device 222 may be configured to capture and process the sensor data based on a device profile that is selected for, provided to, and/or otherwise managed by healthcare data management computing platform 210. For instance, healthcare data management computing platform 210 may select and provide to bedside computing device 222 a device profile, which bedside computing device 222 may then apply and use in capturing and processing sensor data, as discussed above.

In some embodiments, the device profile may include location information associated with the bedside computing device, and the location information may specify a deployment location of the bedside computing device in a healthcare facility. For example, the device profile that may be provided by healthcare data management computing platform 210 to bedside computing device 222 and used by bedside computing device 222 in capturing and processing sensor data may include location information associated with bedside computing device 222, and such location information may specify a deployment location of bedside computing device 222 in a healthcare facility, as discussed in greater detail above.

In some embodiments, the device profile may include service information associated with the bedside computing device, and the service information may specify at least one healthcare service for which the device profile has been customized. For example, the device profile that may be provided by healthcare data management computing platform 210 to bedside computing device 222 and used by bedside computing device 222 in capturing and processing sensor data may include service information associated with bedside computing device 222, and such service information may specify a healthcare service for which bedside computing device 222 has been customized, as discussed in greater detail above.

In some embodiments, the device profile may include network information associated with the bedside computing device, and the network information may specify one or more connection settings to be used by the bedside computing device. For example, the device profile that may be provided by healthcare data management computing platform 210 to bedside computing device 222 and used by bedside computing device 222 in capturing and processing sensor data may include network information associated with bedside computing device 222, and the network information may specify one or more connection settings to be used by bedside computing device 222, as discussed in greater detail above.

In some embodiments, the device profile may include access information associated with the bedside computing device, and the access information may specify one or more authorized users of the bedside computing device. For example, the device profile that may be provided by healthcare data management computing platform 210 to bedside computing device 222 and used by bedside computing device 222 in capturing and processing sensor data may include access information associated with bedside computing device 222, and the access information may specify one or more authorized users of bedside computing device 222, as discussed in greater detail above.

In some embodiments, the device profile may include user interface information associated with the bedside computing device, and the user interface information may specify one or more layout settings to be used by the bedside computing device in presenting one or more graphical user interfaces. For example, the device profile that may be provided by healthcare data management computing platform 210 to bedside computing device 222 and used by bedside computing device 222 in capturing and processing sensor data may include user interface information associated with bedside computing device 222, and the user interface information may specify one or more layout settings to be used by bedside computing device 222, as discussed in greater detail above.

In some embodiments, the device profile may include application information associated with the bedside computing device, and the application information may specify one or more applications that are executable on the bedside computing device. For example, the device profile that may be provided by healthcare data management computing platform 210 to bedside computing device 222 and used by bedside computing device 222 in capturing and processing sensor data may include application information associated with bedside computing device 222, and the application information may specify one or more applications that are executable on and/or by bedside computing device 222, as discussed in greater detail above.

Referring again to FIG. 4C, at step 30, healthcare data management computing platform 210 may receive processed sensor data from bedside computing device 222. For example, at step 30, healthcare data management computing platform 210 may receive the data sent by bedside computing device 222 (e.g., at step 29). In some instances, in addition to receiving processed sensor data from bedside computing device 222 at step 30, healthcare data management computing platform 210 also may perform data filtering on the data received from bedside computing device 222 (e.g., before providing data to EMR server 226, as discussed in greater detail below). In performing such data filtering on the data received from bedside computing device 222, healthcare data management computing platform 210 may, for instance, adapt the data received from bedside computing device 222 for output to one or more other systems (e.g., so as to provide the multi-feed functionality discussed above, for instance, using a DMM included in healthcare data management computing platform 210). At step 31, healthcare data management computing platform 210 may provide data to EMR server 226. For example, at step 31, healthcare data management computing platform 210 may provide any and/or all of the data received from bedside computing device 222 (which may, e.g., include the patient vital signs information that was determined by bedside computing device 222 in processing the captured sensor data) to EMR server 226. At step 32, EMR server 226 may receive the data from healthcare data management computing platform 210. As noted above, the data sent by healthcare data management computing platform 210 and thus received by EMR server 226 may include the patient vital signs information that was determined by bedside computing device 222 in processing the captured sensor data.

Referring to FIG. 4D, at step 33, EMR server 226 may update one or more patient records based on the received data. For example, at step 33, EMR server 226 may update one or more patient records associated with the patient being monitored by bedside computing device 222 so as to include in these records the patient vital signs information that was determined by bedside computing device 222 in processing the captured sensor data.

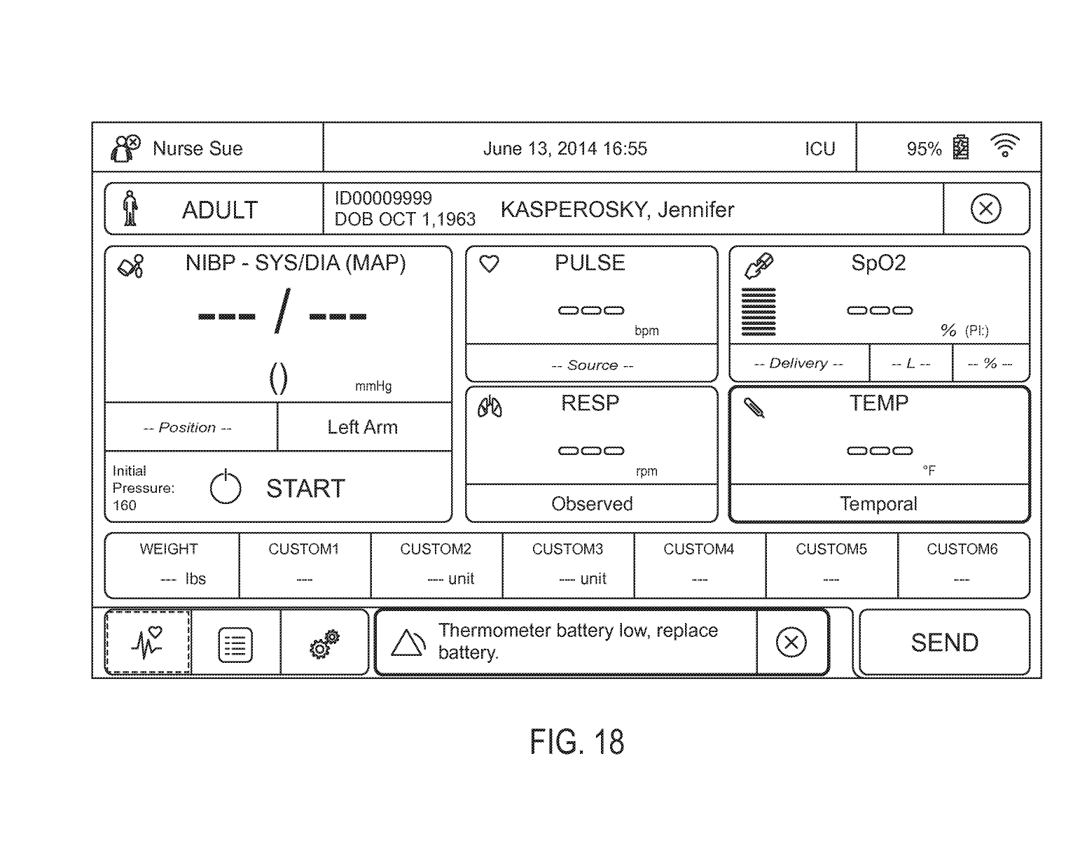

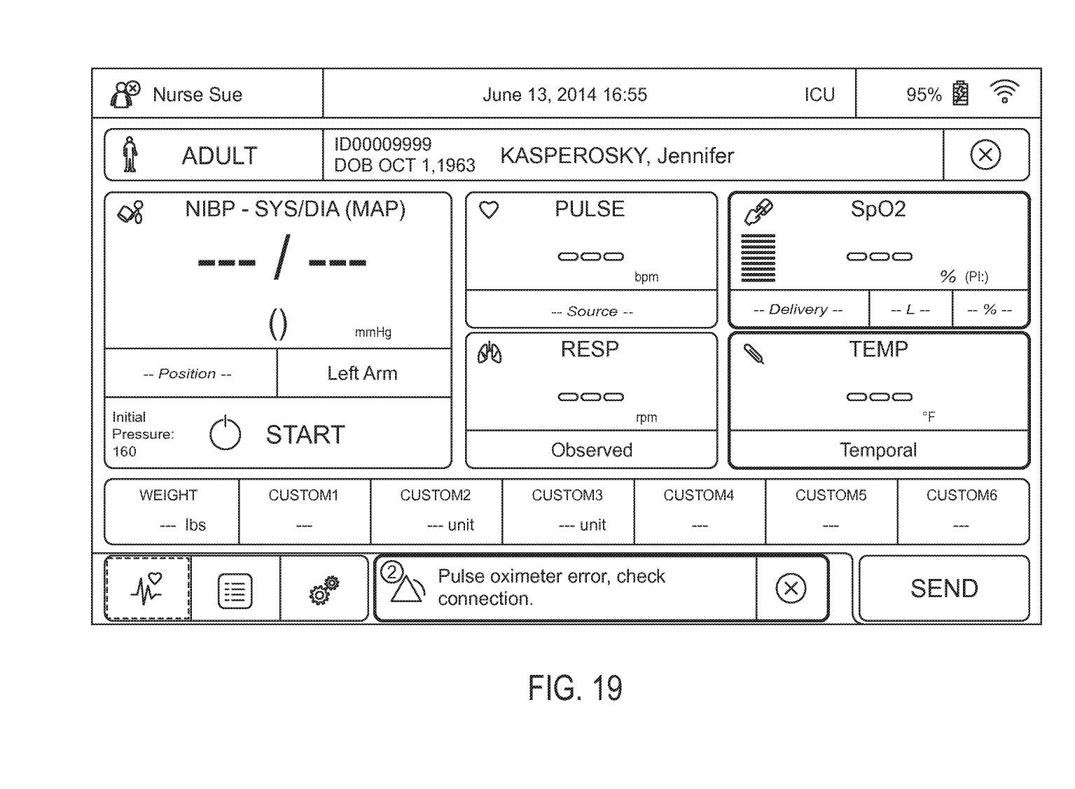

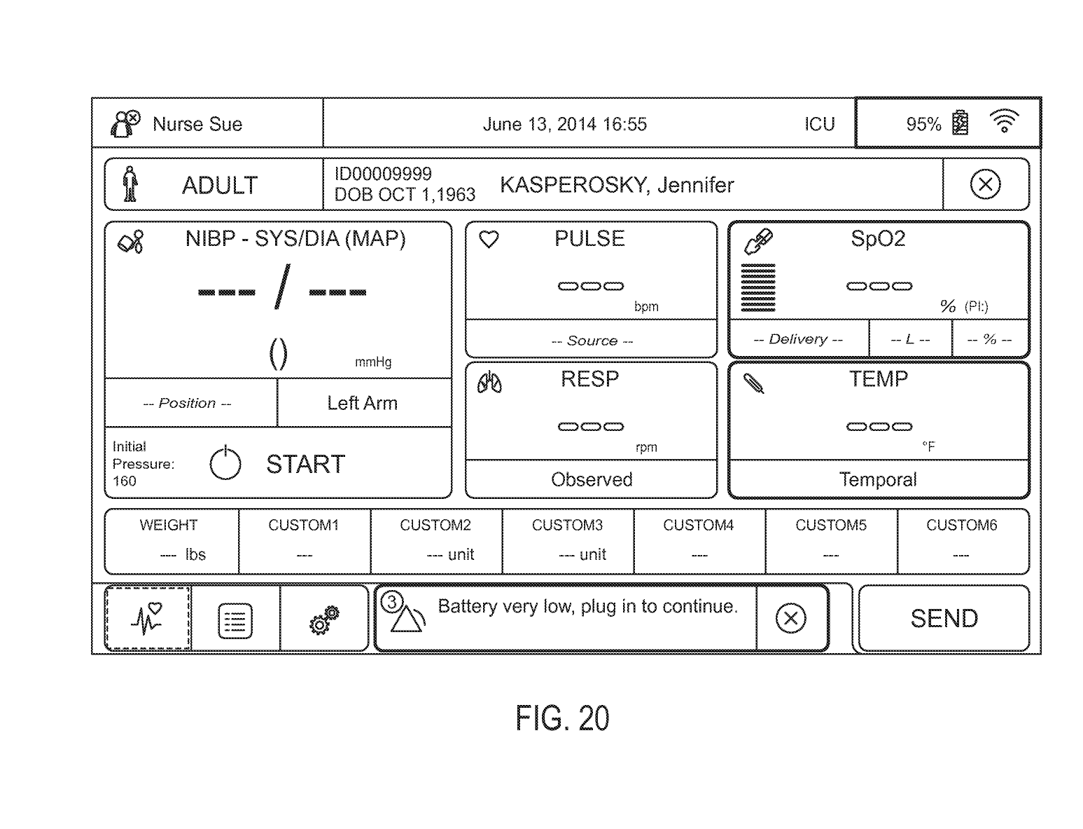

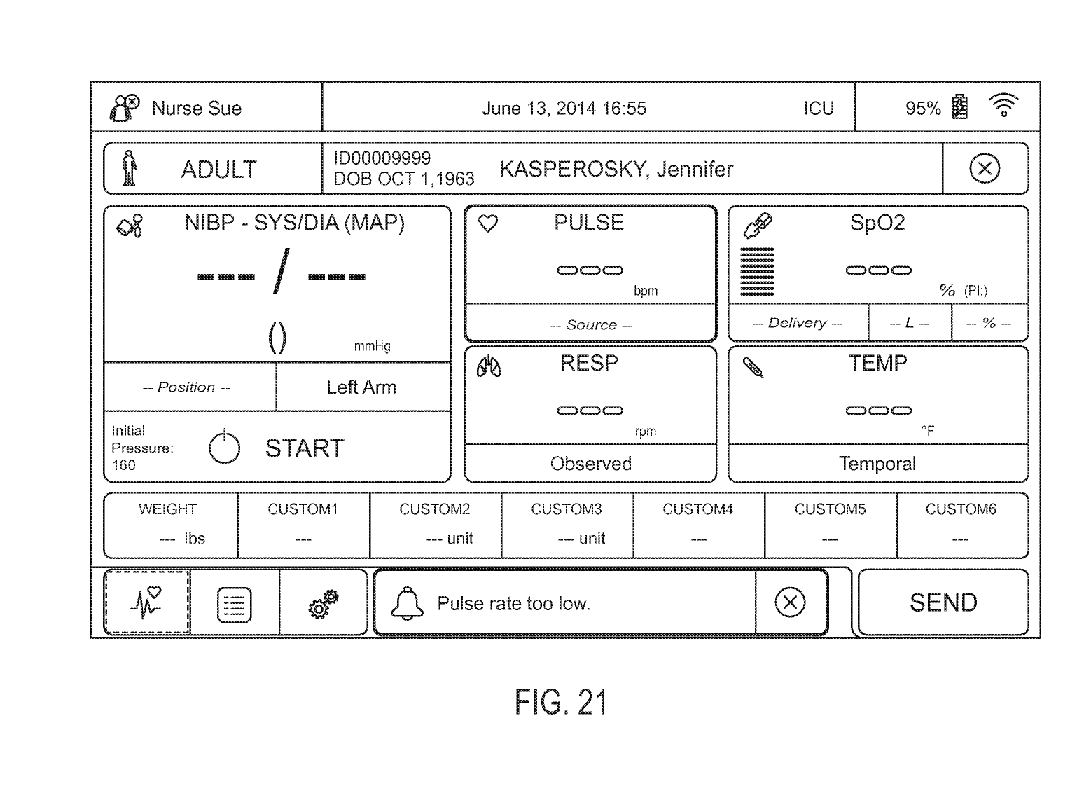

In one or more embodiments, during execution of one or more of the events discussed above, if one or more warnings are detected by bedside computing device 222, bedside computing device 222 may display and/or otherwise present various warning messages. For example, in presenting such a warning message, bedside computing device 222 may generate, display, and/or otherwise present a graphical user interface similar to the example user interface depicted in FIG. 18. As seen in FIG. 18, such a user interface may include a warning message that identifies an issue with a thermometer connected to bedside computing device 222, such as a low battery issue associated with a thermometer connected to bedside computing device 222. Additionally or alternatively, in presenting such a warning message, bedside computing device 222 may generate, display, and/or otherwise present a graphical user interface similar to the example user interface depicted in FIG. 19. As seen in FIG. 19, such a user interface may include a warning message that identifies an issue with a pulse oximeter sensor connected to bedside computing device 222, such as a connection error associated with a pulse oximeter sensor connected to bedside computing device 222. Additionally or alternatively, in presenting such a warning message, bedside computing device 222 may generate, display, and/or otherwise present a graphical user interface similar to the example user interface depicted in FIG. 20. As seen in FIG. 20, such a user interface may include a warning message that identifies an issue with a battery included in bedside computing device 222, such as a low battery issue associated with a battery included in bedside computing device 222. Additionally or alternatively, in presenting such a warning message, bedside computing device 222 may generate, display, and/or otherwise present a graphical user interface similar to the example user interface depicted in FIG. 21. As seen in FIG. 21, such a user interface may include a warning message that identifies an issue with a pulse rate sensor connected to bedside computing device 222, such as a warning message that the detected pulse rate is below a predetermined amount and/or is otherwise considered relatively too low.

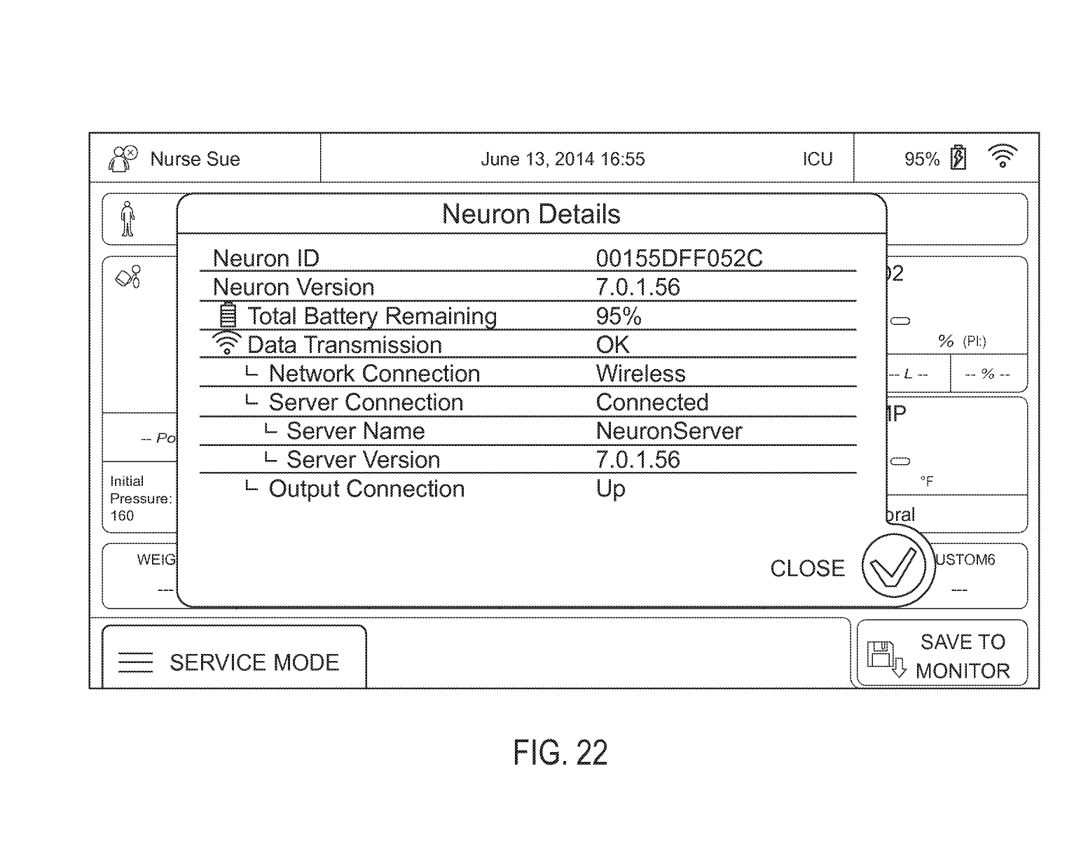

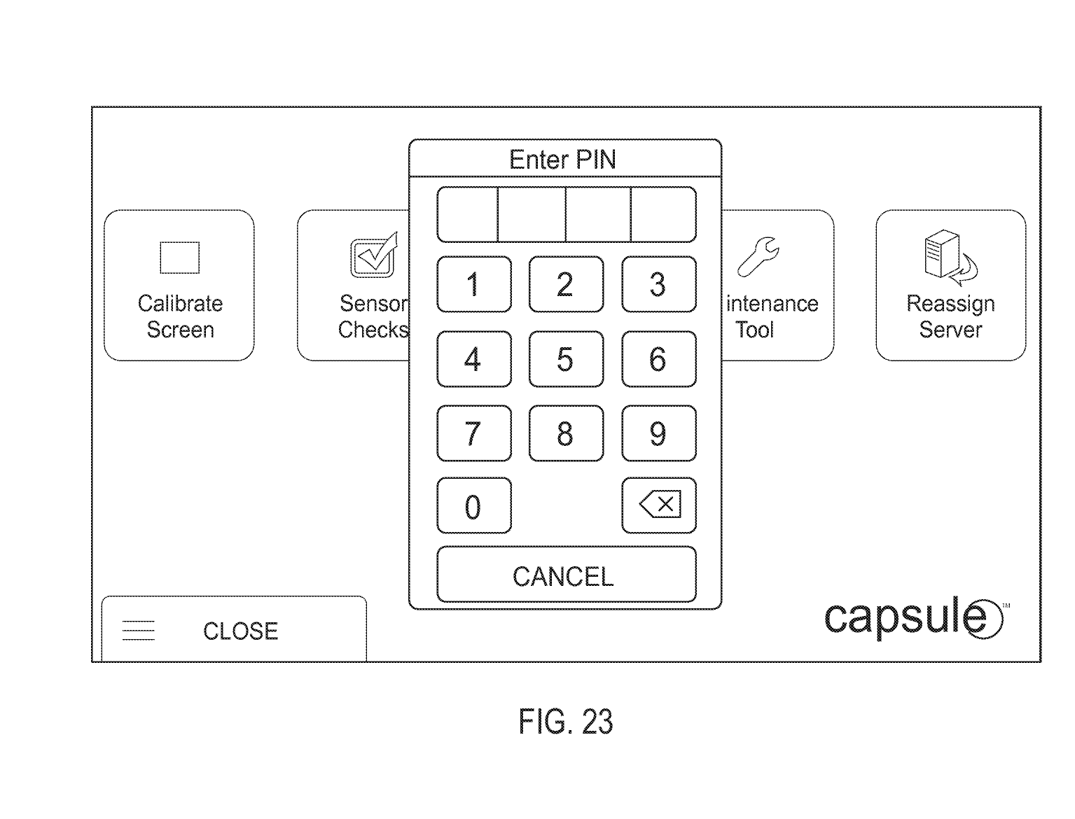

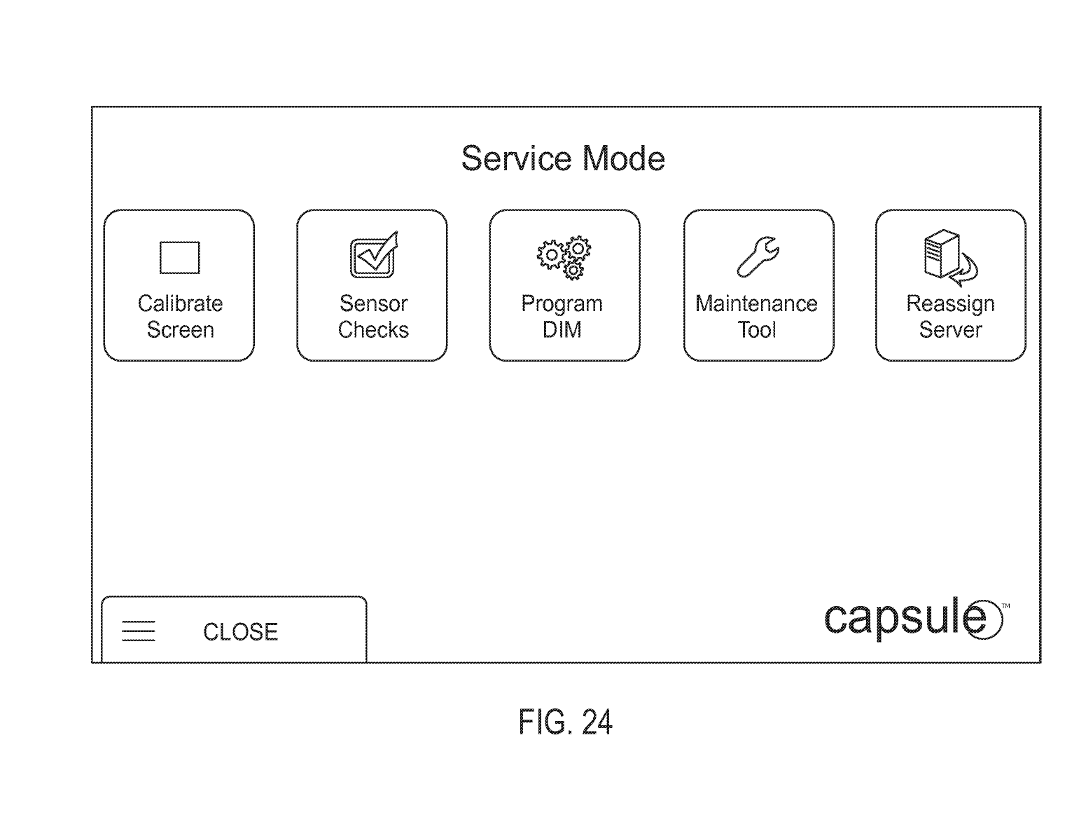

In one or more embodiments, bedside computing device 222 may also display and/or otherwise present various other user interfaces if prompted (e.g., by a user of bedside computing device 222). For example, if prompted to display device configuration details, bedside computing device 222 may generate, display, and/or otherwise present a graphical user interface similar to the example user interface depicted in FIG. 22. As seen in FIG. 22, such a user interface may include information associated with the configuration of bedside computing device 222. As another example, if prompted to display a device service menu, bedside computing device 222 may generate, display, and/or otherwise present a graphical user interface similar to the example user interface depicted in FIG. 23. As seen in FIG. 23, such a user interface may be configured to prompt a user of bedside computing device 222 to enter a passcode which may, for instance, be validated by bedside computing device 222 before a service menu is presented and/or otherwise displayed by bedside computing device 222. Additionally or alternatively, if prompted to display a device service menu, bedside computing device 222 may generate, display, and/or otherwise present a graphical user interface similar to the example user interface depicted in FIG. 24. As seen in FIG. 24, such a user interface may include one or more menu options that enable a user of bedside computing device 222 to perform various functions, such as screen calibration, sensor checks, program configuration, device maintenance, server reassignment, and/or one or more other functions. In addition, in some instances, the example user interface shown in FIG. 24 may be presented after a passcode (which may, e.g., be entered by a user of bedside computing device 222 in response to the prompt included in the example user interface shown in FIG. 23) has been validated.

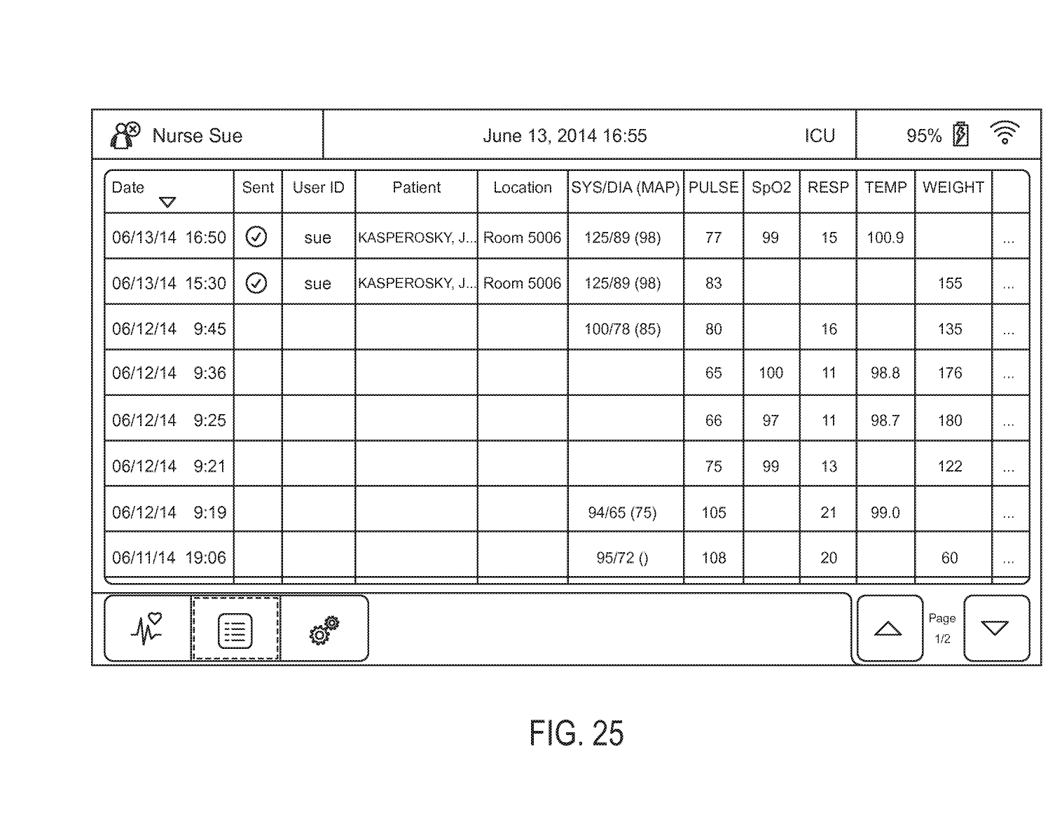

In one or more embodiments, bedside computing device 222 may provide a service mode that may, for instance, enable a user of bedside computing device 222 to define and/or modify various configuration settings associated with bedside computing device 222. In some instances, such a service mode may be accessible to a user of bedside computing device 222 via the service menu illustrated in the example user interface shown in FIG. 24. In some instances, such a service mode may enable bedside computing device 222 to display and/or otherwise present information associated with usage history of bedside computing device 222. In presenting information associated with usage history of bedside computing device 222, bedside computing device 222 may, for example, generate, display, and/or otherwise present a graphical user interface similar to the example user interface depicted in FIG. 25. As seen in FIG. 25, such a user interface may include information about patients that have been monitored by bedside computing device 222, vital signs that have been measured and/or otherwise determined by bedside computing device 222, indications of whether such vital signs have been sent to a central server (e.g., healthcare data management computing platform 210), and/or other information associated with usage of bedside computing device 222. In some arrangements, usage history information (e.g., as shown in FIG. 25) may be accessed from one or more other user interfaces that may be presented by bedside computing device 222, such as from one or more tabs that may be included on a main screen presented by bedside computing device 222 (e.g., rather than via the service mode).

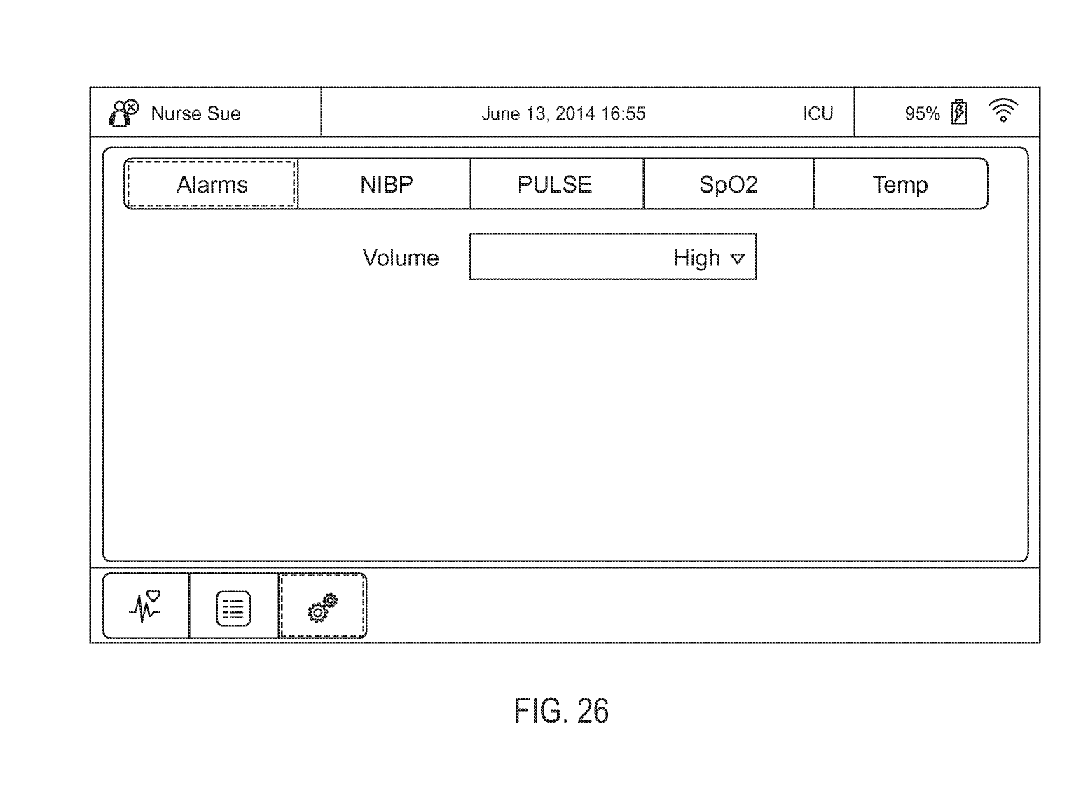

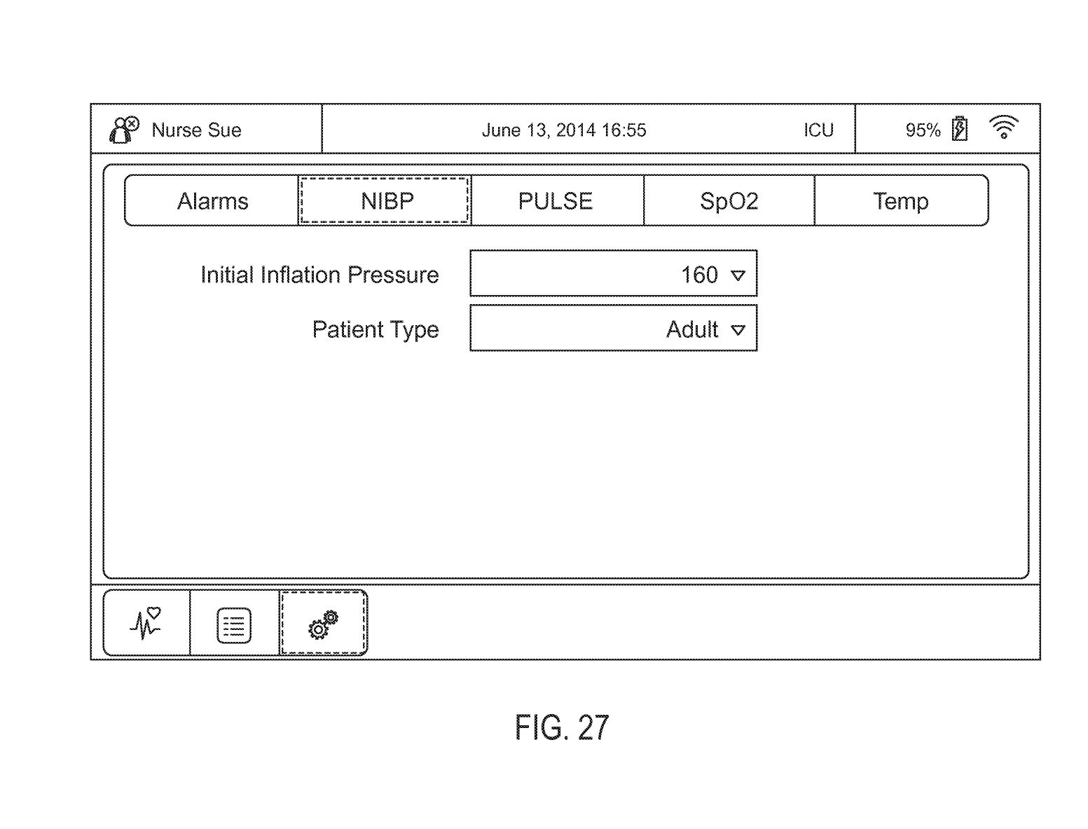

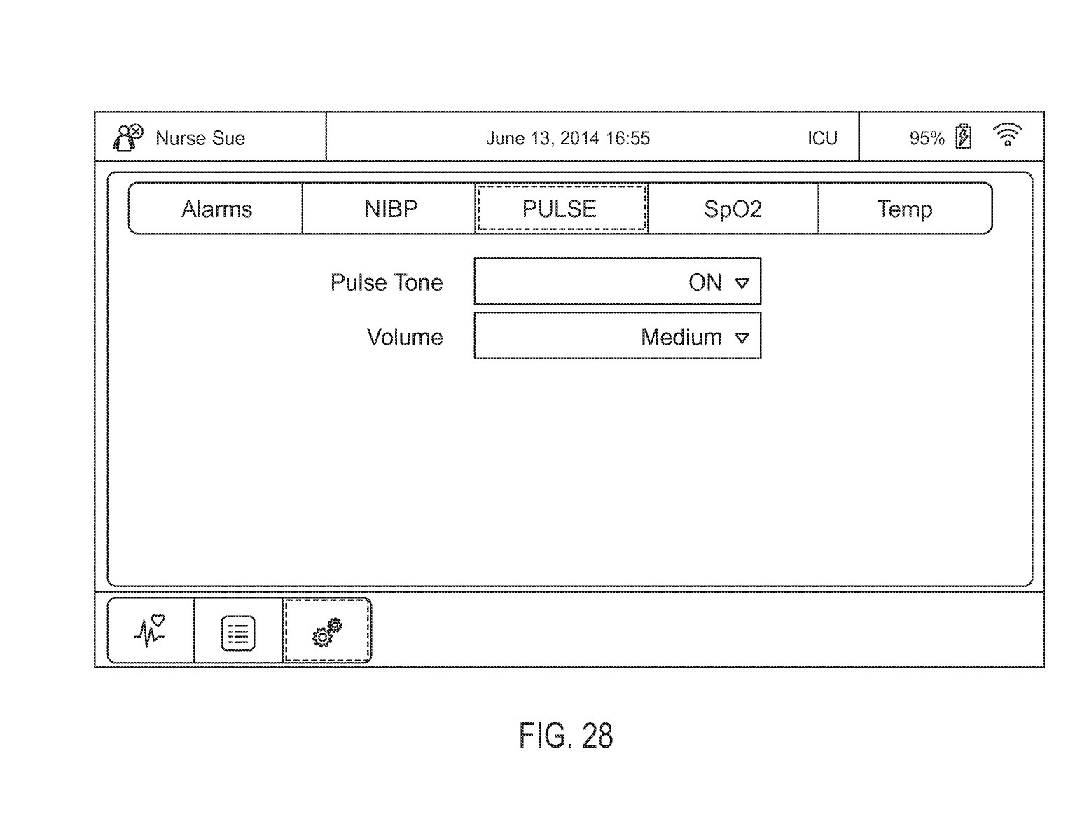

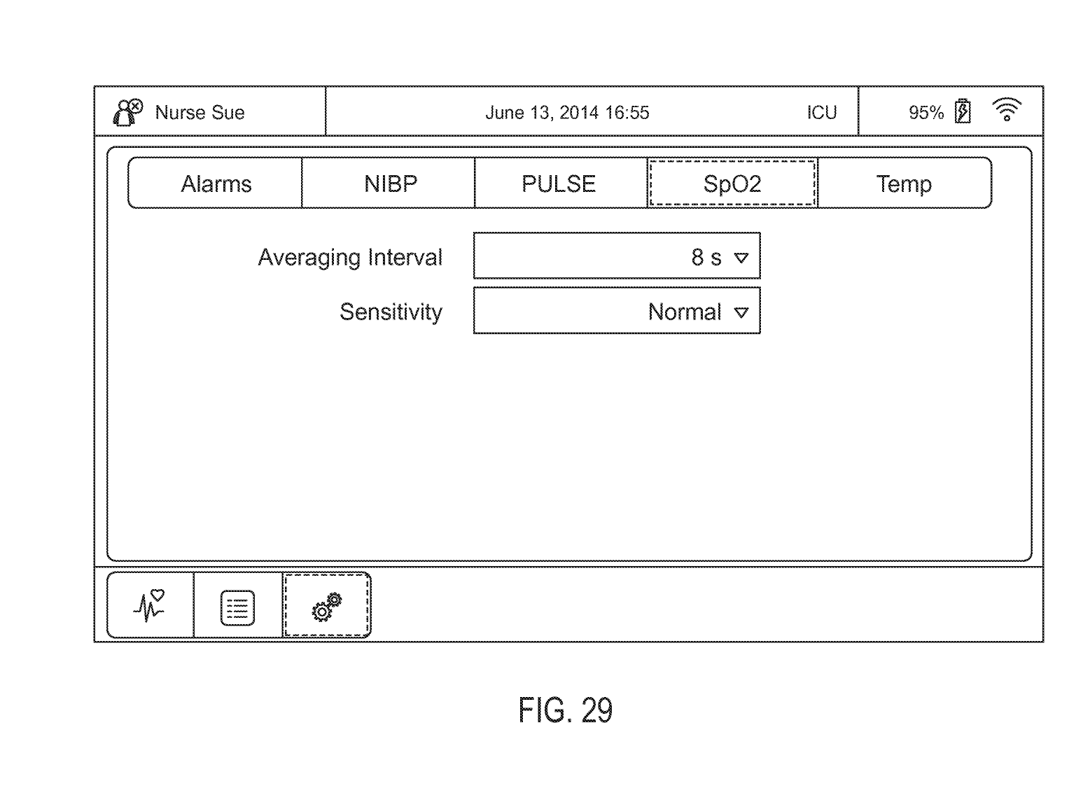

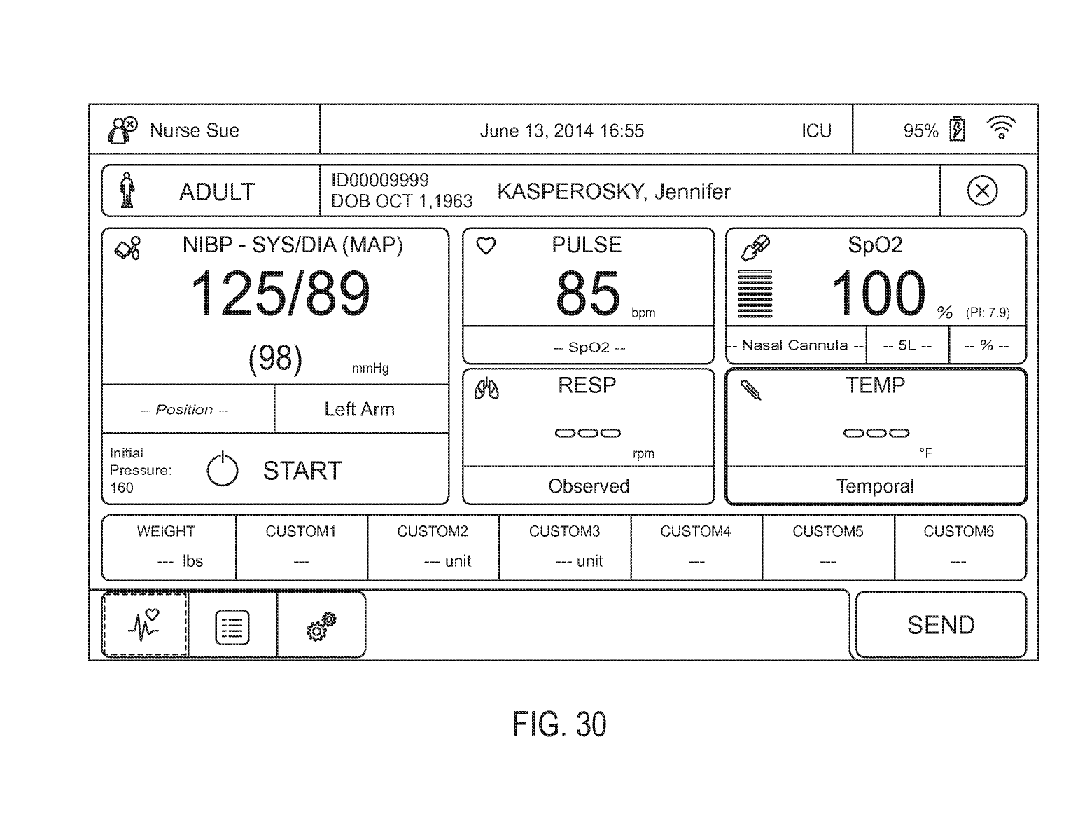

Additionally or alternatively, such a service mode may enable a user of bedside computing device 222 to define and/or modify one or more settings associated with alarms and/or sensors used by bedside computing device 222. In allowing a user to define and/or modify such settings, bedside computing device 222 may generate, display, and/or otherwise present a graphical user interface similar to the example user interface depicted in FIG. 26. As seen in FIG. 26, such a user interface may include one or more controls that enable a user of bedside computing device 222 to define and/or modify one or more settings associated with one or more alarms implemented by bedside computing device 222. Additionally or alternatively, in allowing a user to define and/or modify alarm settings and/or sensor settings, bedside computing device 222 may generate, display, and/or otherwise present a graphical user interface similar to the example user interface depicted in FIG. 27. As seen in FIG. 27, such a user interface may include one or more controls that enable a user of bedside computing device 222 to define and/or modify one or more settings associated with a blood pressure sensor that may be connected to bedside computing device 222. Additionally or alternatively, in allowing a user to define and/or modify alarm settings and/or sensor settings, bedside computing device 222 may generate, display, and/or otherwise present a graphical user interface similar to the example user interface depicted in FIG. 28. As seen in FIG. 28, such a user interface may include one or more controls that enable a user of bedside computing device 222 to define and/or modify one or more settings associated with a pulse rate sensor and/or a pulse oximeter sensor that may be connected to bedside computing device 222. Additionally or alternatively, in allowing a user to define and/or modify alarm settings and/or sensor settings, bedside computing device 222 may generate, display, and/or otherwise present a graphical user interface similar to the example user interface depicted in FIG. 29. As seen in FIG. 29, such a user interface may include one or more controls that enable a user of bedside computing device 222 to define and/or modify one or more settings associated with an oxygen saturation sensor and/or a pulse oximeter sensor that may be connected to bedside computing device 222. Any and/or all of these settings may be used by bedside computing device 222 in operation and may control various aspects of how bedside computing device 222 captures data from one or more sensors, presents measured vital signs information, and/or otherwise functions. For example, when operating, bedside computing device 222 may generate, display, and/or otherwise present a graphical user interface similar to the example user interface depicted in FIG. 30. As seen in FIG. 30, such a user interface may include vital signs information determined by bedside computing device 222 based on sensor data received from various sensors that may be connected to bedside computing device 222 in various ways. In some arrangements, the example user interfaces shown in FIGS. 26-30 may be accessed from one or more other user interfaces that may be presented by bedside computing device 222, such as from one or more tabs that may be included on a main screen presented by bedside computing device 222 (e.g., rather than via the service mode). Additionally or alternatively, the information included in one or more of the user interfaces may be automatically populated based on the one or more sensors that are connected to bedside computing device 222. For example, bedside computing device 222 may detect which sensors and/or types of sensors may be connected to bedside computing device 222, and one or more user interfaces presented by bedside computing device 222 may be dynamically updated based on the detected sensors and/or the detected types of sensors, as well as the configurable settings for such sensors.

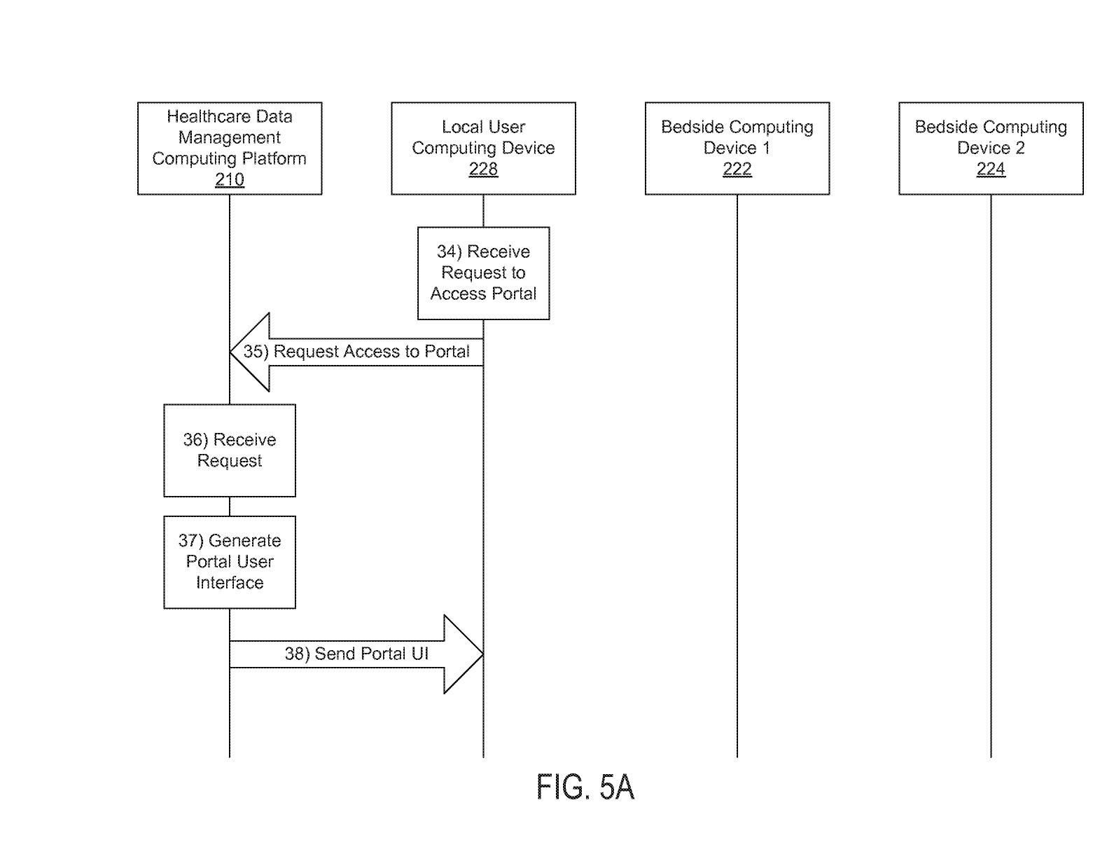

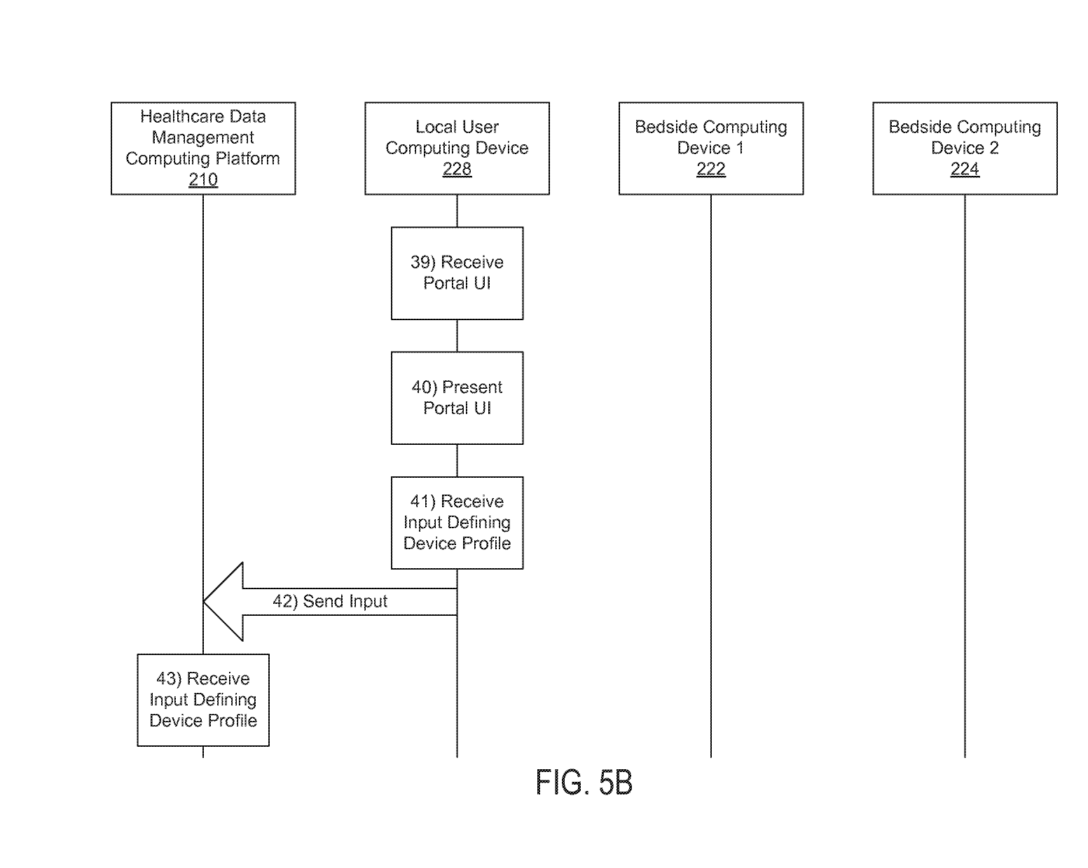

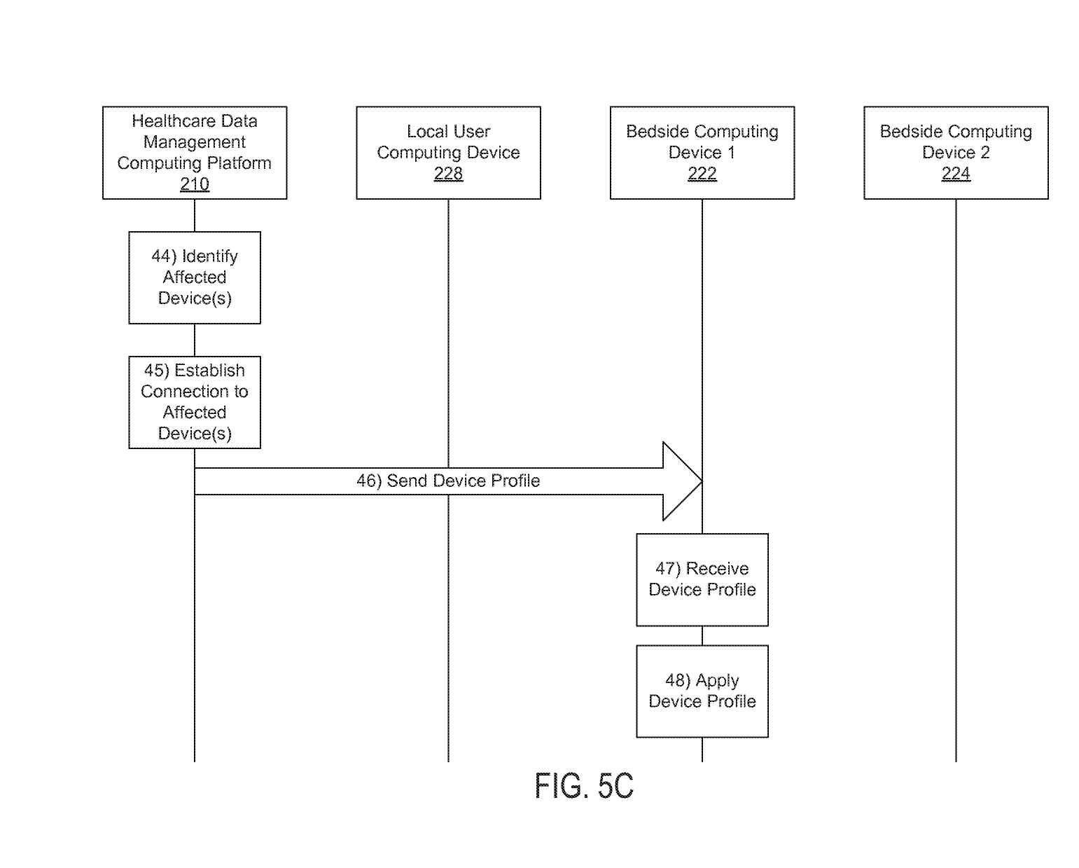

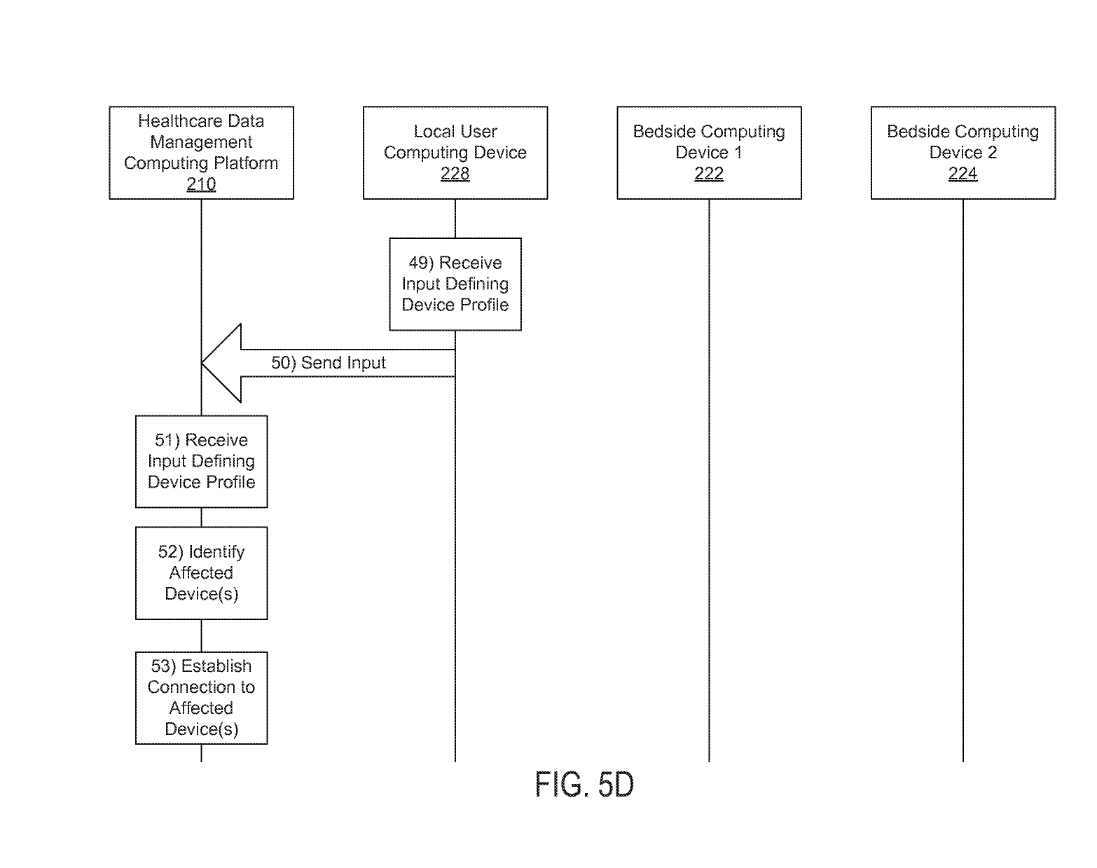

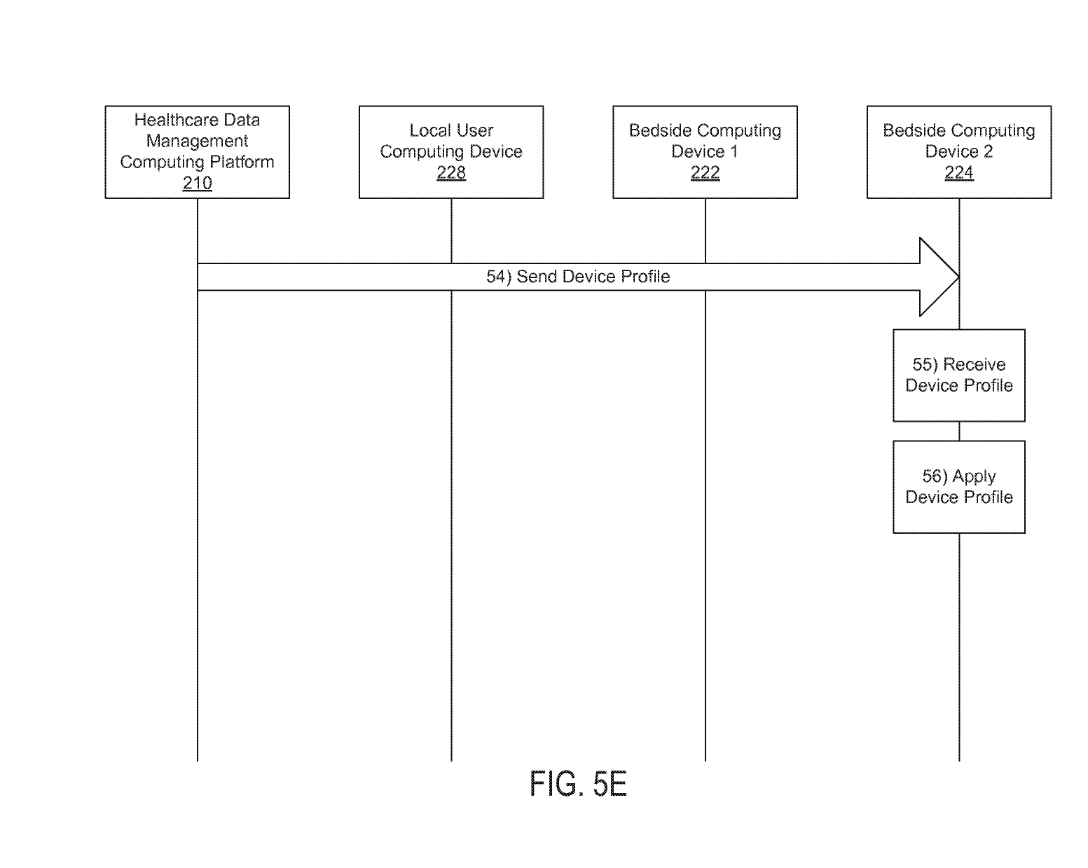

FIGS. 5A-5E depict an example of an event sequence for managing a plurality of bedside computing devices in accordance with one or more illustrative aspects of the disclosure. Referring to FIG. 5A, at step 34, local user computing device 228 may receive a request to access a device management portal (e.g., from a user of local user computing device 228). As discussed in greater detail below, such a device management portal may be provided by healthcare data management computing platform 210 and may enable an administrative user to manage one or more bedside computing devices that may be deployed in a patient care location, such as bedside computing device 222 and bedside computing device 224. At step 35, local user computing device 228 may send a request to access a device management portal to healthcare data management computing platform 210.

At step 36, healthcare data management computing platform 210 may receive a request to access a device management portal. For example, healthcare data management computing platform 210 may receive the request to access the device management portal from local user computing device 228. Based on receiving the request, healthcare data management computing platform 210 may identify and/or authenticate a user of local user computing device 228 to confirm that the user of local user computing device 228 may be provided with access to the device management portal provided by healthcare data management computing platform 210.

At step 37, healthcare data management computing platform 210 may generate a device management portal user interface, and at step 38, healthcare data management computing platform 210 may send the generated device management portal user interface to local user computing device 228. For example, at step 37, after identifying and/or authenticating the user of local user computing device 228, healthcare data management computing platform 210 may generate a device management portal user interface that may include one or more of the example user interfaces discussed in greater detail below. In addition, healthcare data management computing platform 210 may subsequently send the generated portal and/or the generated user interfaces to local user computing device 228 for presentation on local user computing device 228 to a user of local user computing device 228. As noted above, in sending information to one or more other computing devices and/or systems, such as local user computing device 228, healthcare data management computing platform 210 may first encrypt the data that is to be sent before sending such data, and the one or more recipient devices, such as local user computing device 228, may decrypt such data upon receiving it.

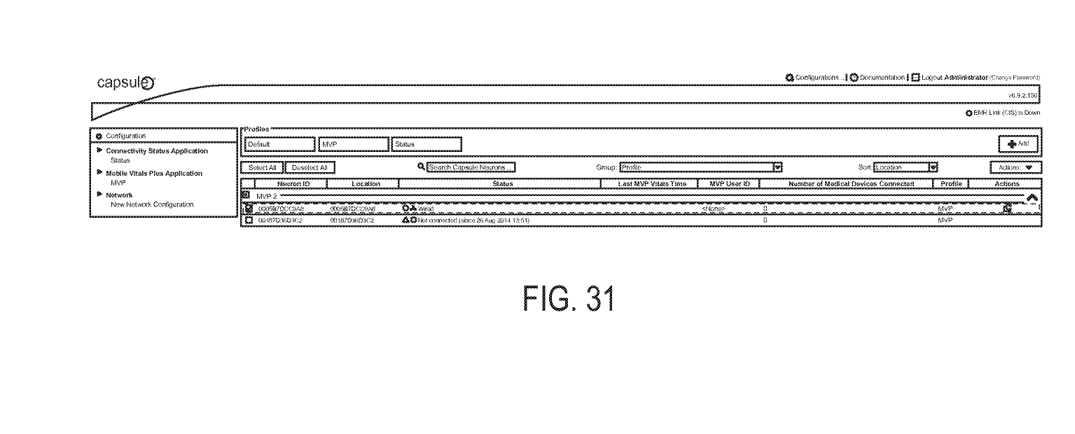

Referring to FIG. 5B, at step 39, local user computing device 228 may receive the device management portal user interface from healthcare data management computing platform 210. At step 40, local user computing device 228 may present the device management portal user interface. In presenting the device management portal user interface, local user computing device 228 may, for example, generate, display, and/or otherwise present a graphical user interface similar to the example user interface depicted in FIG. 31. As seen in FIG. 31, such a user interface may include information associated with one or more bedside computing devices being managed by healthcare data management computing platform 210 and/or one or more controls that enable a user of local user computing device 228 to define and/or modify configuration settings for the one or more bedside computing devices being managed by healthcare data management computing platform 210. For example, and as seen in FIG. 31, the user interface may include device identifier information for the one or more bedside computing devices being managed by healthcare data management computing platform 210, location information for the one or more bedside computing devices being managed by healthcare data management computing platform 210, status information for the one or more bedside computing devices being managed by healthcare data management computing platform 210, user information for the one or more bedside computing devices being managed by healthcare data management computing platform 210, sensor and/or medical device information for the one or more bedside computing devices being managed by healthcare data management computing platform 210, profile information for the one or more bedside computing devices being managed by healthcare data management computing platform 210, and/or other information associated with the one or more bedside computing devices being managed by healthcare data management computing platform 210.

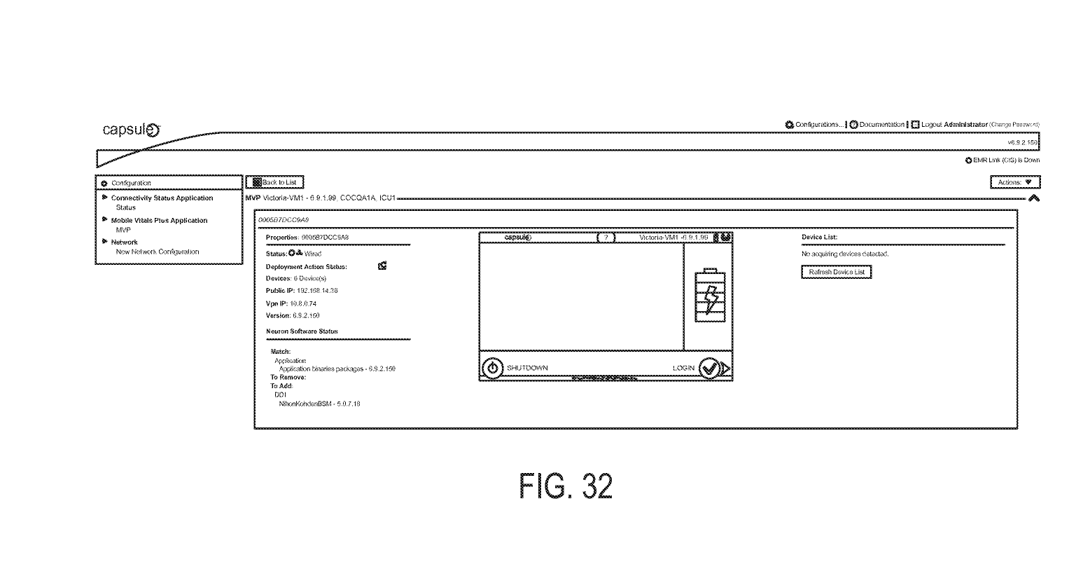

Additionally or alternatively, in presenting the device management portal user interface, local user computing device 228 may generate, display, and/or otherwise present a graphical user interface similar to the example user interface depicted in FIG. 32. As seen in FIG. 32, such a user interface may include information associated with a specific bedside computing device being managed by healthcare data management computing platform 210 and/or one or more controls that enable a user of local user computing device 228 to define and/or modify configuration settings for the specific bedside computing device. For example, and as seen in FIG. 32, the user interface may include device identifier information for the bedside computing device, connection status information for the bedside computing device, deployment action information for the bedside computing device, information associated with device(s) connected to the bedside computing device, network information associated with the bedside computing device (e.g., a public network address, a VPN address, etc.), version information associated with the bedside computing device, and/or software status information associated with the bedside computing device (which may, e.g., include version information and/or other information associated with one or more applications that are running on and/or executable on the bedside computing device).

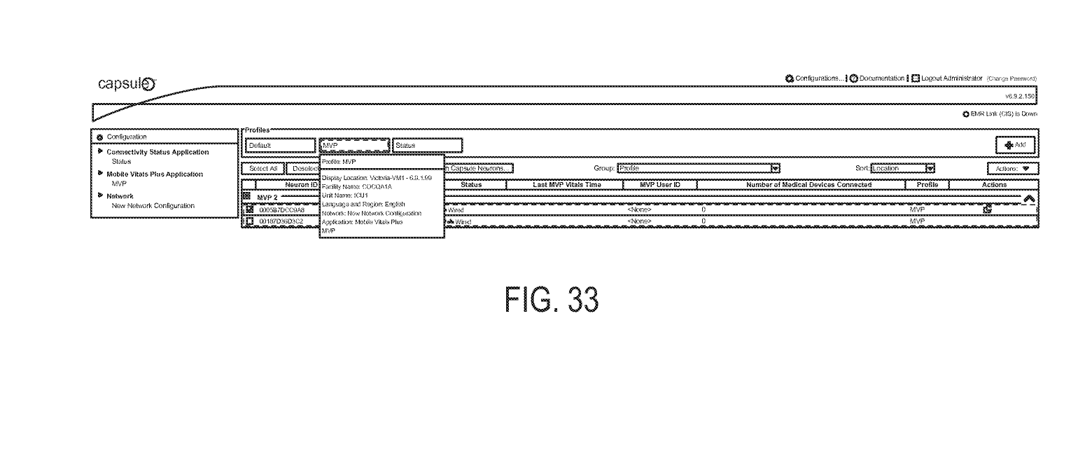

Additionally or alternatively, in presenting the device management portal user interface, local user computing device 228 may generate, display, and/or otherwise present a graphical user interface similar to the example user interface depicted in FIG. 33. As seen in FIG. 33, such a user interface may include profile information associated with one or more bedside computing devices being managed by healthcare data management computing platform 210 and/or one or more controls that enable a user of local user computing device 228 to define and/or modify one or more device profiles that may be used by and/or applied to a set of bedside computing devices, such as bedside computing device 222 and/or bedside computing device 224. For example, and as seen in FIG. 33, the user interface may include a profile name associated with the device profile, a location associated with the device profile, a facility name associated with the device profile, a unit name associated with the device profile, a language and/or region associated with the device profile, a network configuration associated with the device profile, one or more applications associated with the device profile, and/or other information associated with the device profile. In addition, the user interface may include one or more controls for defining and/or modifying various aspects of the device profile, as well as one or more controls for applying the device profile to one or more bedside computing devices that may be managed by healthcare data management computing platform 210, such as bedside computing device 222 and/or bedside computing device 224.

Referring again to FIG. 5B, at step 41, local user computing device 228 may receive input defining a device profile for a bedside computing device. For example, at step 40, local user computing device 228 may receive input defining a device profile to be applied to one or more bedside computing devices managed by healthcare data management computing platform 210, such as bedside computing device 222 and/or bedside computing device 224. In one or more embodiments, such input may be received via one or more of the example user interfaces discussed above and shown in FIGS. 31-33.

Referring again to FIG. 5B, at step 42, local user computing device 228 may send the input defining the device profile to healthcare data management computing platform 210. At step 43, healthcare data management computing platform 210 may receive the input defining the device profile from local user computing device 228. For example, at step 43, healthcare data management computing platform 210 may receive (e.g., from local user computing device 228) input defining a first device profile for a first set of one or more bedside computing devices. For purposes of this example, the first set of one or more bedside computing devices may include bedside computing device 222.

Referring to FIG. 5C, at step 44, healthcare data management computing platform 210 may identify one or more bedside computing devices for which the first device profile applies (e.g., based on their inclusion in the first set of one or more bedside computing devices). For example, at step 44, healthcare data management computing platform 210 may determine that the first device profile applies to bedside computing device 222.

At step 45, healthcare data management computing platform 210 may establish a network connection to at least one bedside computing device included in the first set of one or more bedside computing devices. For example, at step 45, healthcare data management computing platform 210 may establish a connection with bedside computing device 222. In some instances, healthcare data management computing platform 210 may initiate the connection with a bedside computing device, such as bedside computing device 222, while in other instances, a bedside computing device, such as bedside computing device 222, may initiate the connection with healthcare data management computing platform 210. Additionally or alternatively, healthcare data management computing platform 210 and/or the bedside computing device, such as bedside computing device 222, may keep the connection open when possible. In instances where such a connection is kept open and a device profile is modified and/or otherwise updated, the one or more bedside computing devices that may be connected to healthcare data management computing platform 210 and/or affected by the modified device profile may be notified, and the updated device profile may be pushed by healthcare data management computing platform 210 to the bedside computing devices and applied.

At step 46, healthcare data management computing platform 210 may provide the first device profile to the at least one bedside computing device with which the network connection was established. For example, at step 46, healthcare data management computing platform 210 may send the first device profile to bedside computing device 222.

In one or more embodiments, the server computing device may be configured to interface with at least one electronic medical records (EMR) system for logging of patient vital signs information received from the first set of one or more bedside computing devices. In interfacing with the EMR system and/or with one or more other computer systems, the server computing device may, for example, send, receive, and/or otherwise exchange information with such other computer systems. For example, healthcare data management computing platform 210 may be configured to interface with an EMR system, such as EMR server 226, as discussed in greater detail above, for logging of patient vital signs information received from the first set of one or more bedside computing devices (which may, e.g., include bedside computing device 222). Additionally or alternatively, the server computing device may be configured to interface with at least one central monitoring system for live monitoring of patient vital signs information received from the first set of one or more bedside computing devices. For example, healthcare data management computing platform 210 may be configured to interface with a central monitoring system (which may, e.g., be deployed at a central location with a patient care environment, such as a nurse station, to facilitate simultaneous monitoring of multiple patients in the patient care environment, which may, e.g., be a particular floor or ward of a hospital) that is configured to provide live monitoring of patient vital signs received from bedside computing device 222 and/or one or more other bedside computing devices included in the first set of one or more bedside computing devices to which the first device profile is applicable.

Additionally or alternatively, the server computing device may be configured to interface with at least one directory system for identifying one or more users of the one or more bedside computing devices. For example, healthcare data management computing platform 210 may be configured to interface with a directory system, such as an Active Directory system or a lightweight directory access protocol (LDAP) system to facilitate identification of one or more users of the one or more bedside computing devices. Such a directory system may, for instance, store and/or maintain contact information associated with nurses, doctors, and other healthcare providers associated with a healthcare organization, and by interfacing with such a directory system, healthcare data management computing platform 210 may enable a user of a healthcare data management computing platform to quickly and easily identify themselves for purposes of tracking which healthcare providers are interacting with the particular bedside computing device and/or using the bedside computing device to capture data associated with a particular patient. Additionally or alternatively, the one or more bedside computing devices may be deployed in a patient care environment associated with a healthcare facility. For example, the one or more bedside computing devices (e.g., bedside computing device 222) may be deployed in a particular clinic, floor, or ward of a hospital.

At step 47, bedside computing device 222 may receive the device profile from healthcare data management computing platform 210, and at step 48, bedside computing device 222 may apply the device profile received from healthcare data management computing platform 210. In applying the device profile received from healthcare data management computing platform 210, bedside computing device 222 may, for example, define and/or modify one or more operating settings and/or other configuration settings that may be used by bedside computing device 222 based on the information and/or other settings included in the device profile received from healthcare data management computing platform 210, as discussed above.

In some embodiments, the first device profile may include location information associated with the first set of one or more bedside computing devices, and the location information may specify a deployment location of the first set of one or more bedside computing devices in a healthcare facility. For example, the device profile that is sent to bedside computing device 222 by healthcare data management computing platform 210 and applied by bedside computing device 222 may include location information associated with bedside computing device 222, and the location information may specify a deployment location of bedside computing device 222 within a healthcare facility, as discussed in greater detail above.

In some embodiments, the first device profile may include service information associated with the first set of one or more bedside computing devices, and the service information may specify at least one healthcare service for which the first device profile has been customized. For example, the device profile that is sent to bedside computing device 222 by healthcare data management computing platform 210 and applied by bedside computing device 222 may include service information associated with bedside computing device 222, and the service information may specify a healthcare service for which the device profile has been customized, as discussed in greater detail above.

In some embodiments, the first device profile may include network information associated with the first set of one or more bedside computing devices, and the network information may specify one or more connection settings to be used by the first set of one or more bedside computing devices. For example, the device profile that is sent to bedside computing device 222 by healthcare data management computing platform 210 and applied by bedside computing device 222 may include network information associated with bedside computing device 222, and the network information may specify one or more connection settings to be used by the first set of bedside computing devices (which may, e.g., include bedside computing device 222), as discussed in greater detail above.

In some embodiments, the first device profile may include access information associated with the first set of one or more bedside computing devices, and the access information may specify one or more authorized users of the first set of one or more bedside computing devices. For example, the device profile that is sent to bedside computing device 222 by healthcare data management computing platform 210 and applied by bedside computing device 222 may include access information associated with bedside computing device 222, and the access information may specify one or more authorized users of the first set of bedside computing devices (which may, e.g., include bedside computing device 222), as discussed in greater detail above.

In some embodiments, the first device profile may include user interface information associated with the first set of one or more bedside computing devices, and the user interface information may specify one or more layout settings to be used by the first set of one or more bedside computing devices in presenting one or more graphical user interfaces. For example, the device profile that is sent to bedside computing device 222 by healthcare data management computing platform 210 and applied by bedside computing device 222 may include user interface information associated with bedside computing device 222, and the user interface information may specify one or more layout settings to be used by the first set of bedside computing devices (which may, e.g., include bedside computing device 222), as discussed in greater detail above.

In some embodiments, the first device profile may include application information associated with the first set of one or more bedside computing devices, and the application information may specify one or more applications that are executable on the first set of one or more bedside computing devices. For example, the device profile that is sent to bedside computing device 222 by healthcare data management computing platform 210 and applied by bedside computing device 222 may include application information associated with bedside computing device 222, and the application information may specify one or more applications that are executable by and/or on the first set of bedside computing devices (which may, e.g., include bedside computing device 222), as discussed in greater detail above.

Referring to FIG. 5D, at step 49, local user computing device 228 may receive input defining an updated device profile for one or more bedside computing devices that are managed by healthcare data management computing platform 210. Such input may, for instance, define and/or modify a device profile for a second set of bedside computing devices different from the first set of bedside computing devices for which the first device profile was defined. In addition, such input may, for example, be received from a user of local user computing device 228 via one or more of the example user interfaces discussed above and shown in FIGS. 31-33.

Referring again to FIG. 5D, at step 50, local user computing device 228 may send the input defining the updated device profile to healthcare data management computing platform 210. At step 51, healthcare data management computing platform 210 may receive the input defining the updated device profile from local user computing device 228. For example, at step 51, healthcare data management computing platform 210 may receive (e.g., from local user computing device 228) input defining a second, updated device profile for a second set of one or more bedside computing devices. For purposes of this example, the second set of one or more bedside computing devices may include bedside computing device 224.

At step 52, healthcare data management computing platform 210 may identify one or more bedside computing devices for which the second device profile applies (e.g., based on their inclusion in the second set of one or more bedside computing devices). For example, at step 52, healthcare data management computing platform 210 may determine that the second device profile applies to bedside computing device 224.

At step 53, healthcare data management computing platform 210 may establish a network connection to at least one second bedside computing device included in the second set of one or more bedside computing devices. For example, at step 53, healthcare data management computing platform 210 may establish a connection with bedside computing device 224.

Referring to FIG. 5E, at step 54, healthcare data management computing platform 210 may provide the second, updated device profile to the at least one second bedside computing device with which the network connection was established. For example, at step 54, healthcare data management computing platform 210 may send the second device profile to bedside computing device 224. The second, updated device profile that is provided to bedside computing device 224 by healthcare data management computing platform 210 may, for example, include similar information as the first device profile that is provided to bedside computing device 222 by healthcare data management computing platform 210. For example, the second, updated profile that is provided to bedside computing device 224 by healthcare data management computing platform 210 may, for example, include location information associated with the second set of one or more bedside computing devices, service information associated with the second set of one or more bedside computing devices, network information associated with the second set of one or more bedside computing devices, access information associated with the second set of one or more bedside computing devices, user interface information associated with the second set of one or more bedside computing devices, and/or application information associated with the second set of one or more bedside computing devices.

In some embodiments, providing the updated device profile to the at least one second bedside computing device may include providing a firmware update to the at least one second bedside computing device. For example, in providing the updated device profile to bedside computing device 224, healthcare data management computing platform 210 may, in some instances, provide a firmware update to bedside computing device 224. Such a firmware update may, for example, be configured to cause bedside computing device 224 to install and/or otherwise use new firmware that may be defined by and/or included in the firmware update provided by healthcare data management computing platform 210.

At step 55, bedside computing device 224 may receive the device profile from healthcare data management computing platform 210, and at step 56, bedside computing device 224 may apply the device profile received from healthcare data management computing platform 210. In applying the device profile received from healthcare data management computing platform 210, bedside computing device 224 may, for example, define and/or modify one or more operating settings and/or other configuration settings that may be used by bedside computing device 224 based on the information and/or other settings included in the device profile received from healthcare data management computing platform 210, as discussed above

While several of the events discussed above involve local user computing device 228 interacting with healthcare data management computing platform 210, in additional and/or alternative embodiments, one or more different computing devices, such as remote user computing device 240, may similarly interact with healthcare data management computing platform 210 to define one or more device profiles that may be provided by healthcare data management computing platform 210 to one or more bedside computing devices, such as bedside computing device 222 and bedside computing device 224, and subsequently applied by such bedside computing devices.

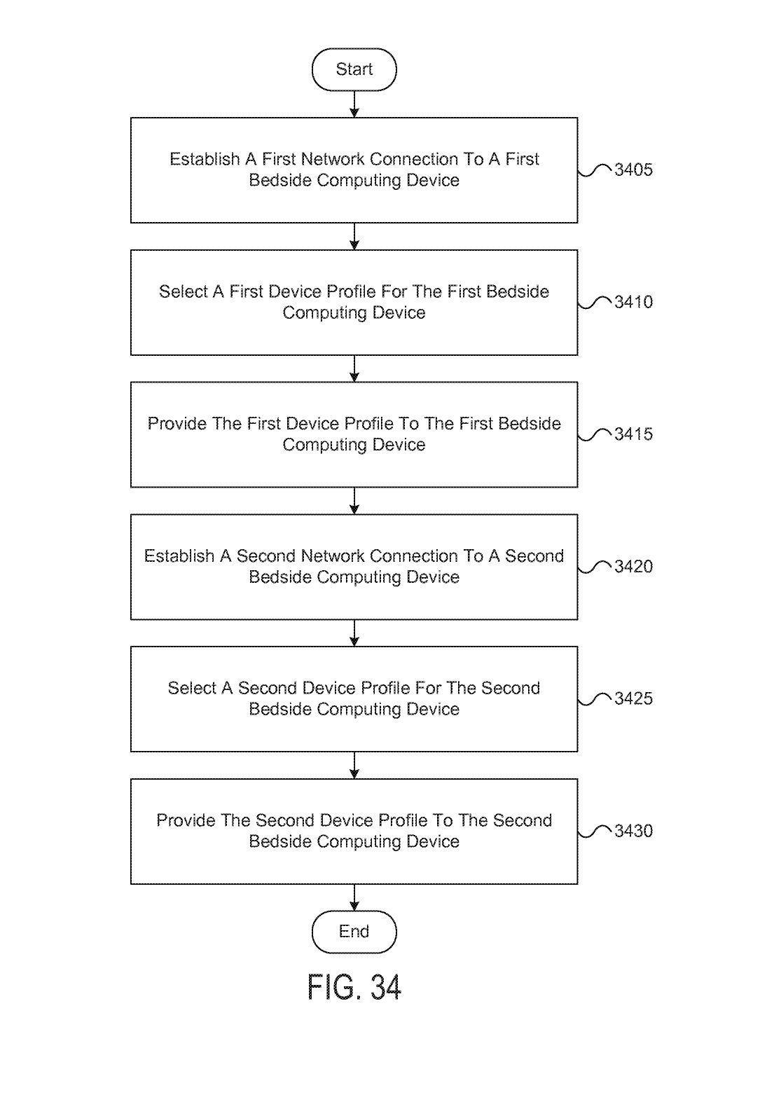

FIG. 34 depicts an example of a method of configuring a plurality of bedside computing devices in accordance with one or more illustrative aspects of the disclosure. Referring to FIG. 34, at step 3405, a server computing device may establish a first network connection to a first bedside computing device. At step 3410, the server computing device may select a first device profile for the first bedside computing device. At step 3415, the server computing device may provide the first device profile to the first bedside computing device. At step 3420, the server computing device may establish a second network connection to a second bedside computing device different from the first bedside computing device. At step 3425, the server computing device may select a second device profile for the second bedside computing device. At step 3430, the server computing device may provide the second device profile to the second bedside computing device.

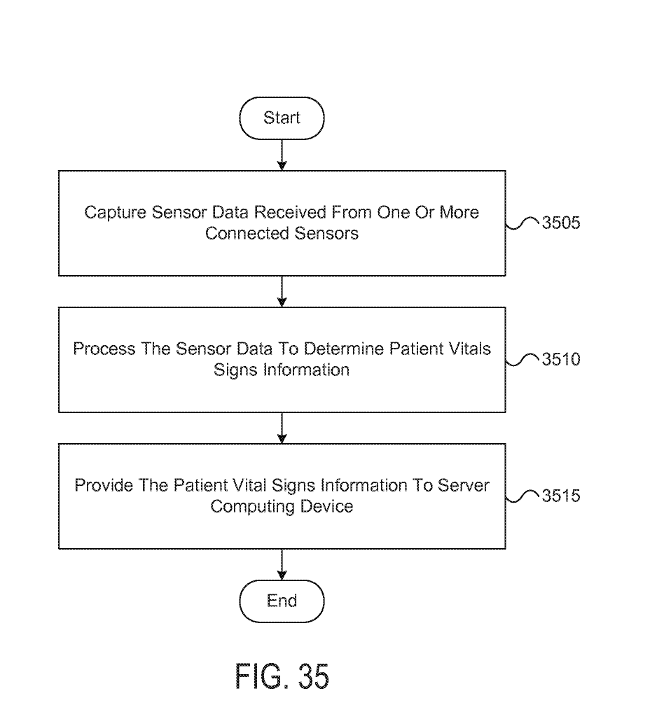

FIG. 35 depicts an example of a method of capturing and processing sensor data in accordance with one or more illustrative aspects of the disclosure. Referring to FIG. 35, at step 3505, a first bedside computing device may capture sensor data received from one or more sensors connected to the first bedside computing device. At step 3510, the first bedside computing device may process the sensor data to determine patient vital signs information. At step 3515, the first bedside computing device may provide the patient vital signs information to a server computing device that is configured to receive vital signs information from one or more additional bedside computing devices different from the first bedside computing device.

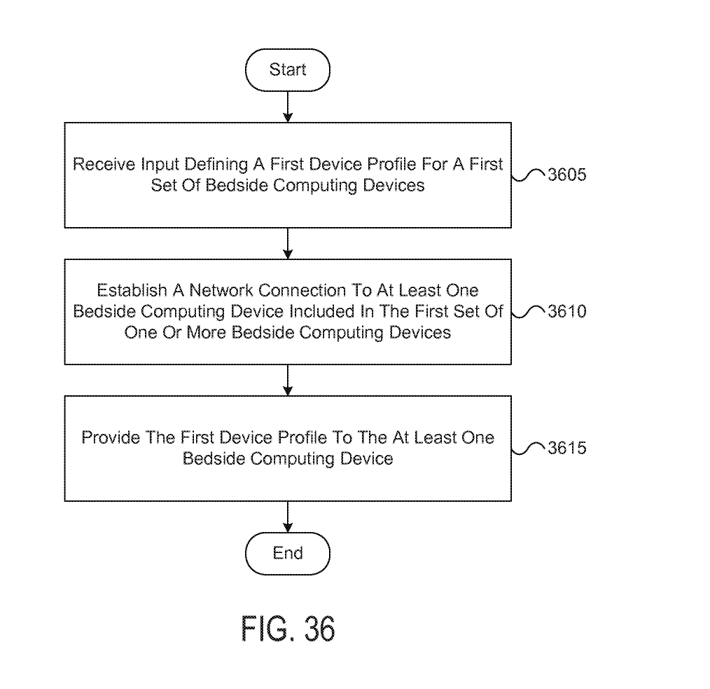

FIG. 36 depicts an example of a method of managing a plurality of bedside computing devices in accordance with one or more illustrative aspects of the disclosure. Referring to FIG. 36, at step 3605, a server computing device may receive input defining a first device profile for a first set of one or more bedside computing devices. At step 3610, the server computing device may establish a network connection to at least one bedside computing device included in the first set of one or more bedside computing devices. At step 3615, the server computing device may provide the first device profile to the at least one bedside computing device.

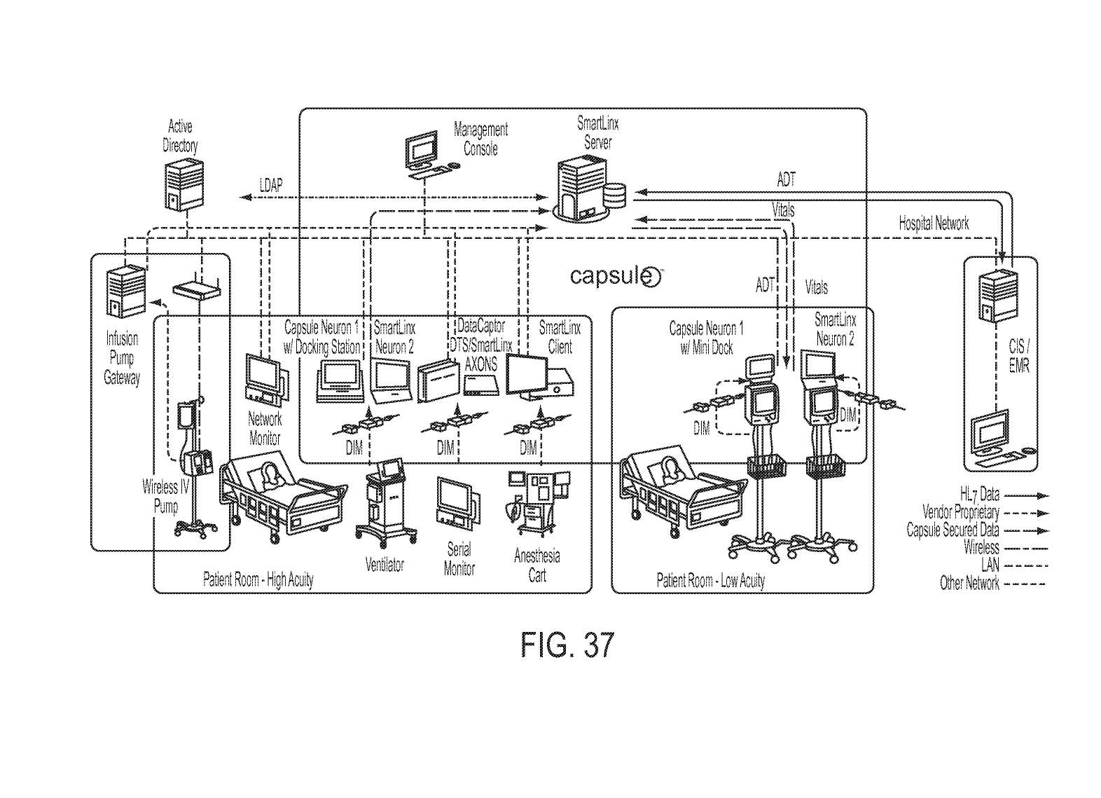

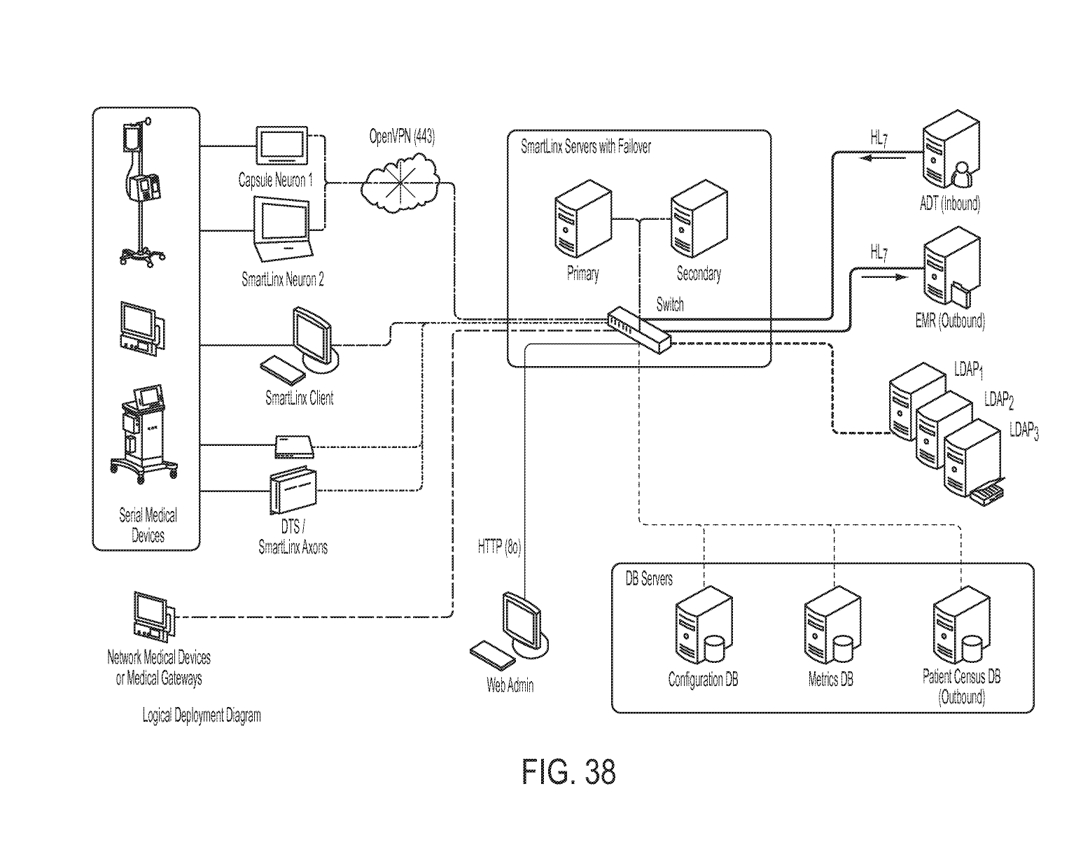

FIGS. 37 and 38 depict additional examples of computing environments for capturing and managing healthcare information in accordance with one or more illustrative aspects of the disclosure. For instance, in the examples illustrated in FIGS. 37 and 38, the “Neurons” may incorporate one or more aspects of the bedside computing devices discussed above, such as bedside computing device 222 and bedside computing device 224. In addition, in the examples illustrated in FIGS. 37 and 38, the “SmartLinx Server” may incorporate one or more aspects of the healthcare data management computing platform 210 discussed above. As shown in FIGS. 37 and 38, the Neurons may be connected directly to one or more sensors in some instances, while in other instances the Neurons may be connected to one or more sensors via one or more medical devices. In addition, the Neurons may provide filtered and/or processed sensor data to the SmartLinx server, which may interface with an EMR server and other computer systems that may be associated with a healthcare organization. For example, as shown in FIG. 37, a Neuron and a SmartLinx server may be deployed in a high acuity environment in which the Neuron and SmartLinx server interface with a number of other sensors and medical devices, including vents, infusion pumps, monitors, pulse oximeters, and/or other systems and devices. In another example also shown in FIG. 37, a Neuron and a SmartLinx server may be deployed in a low acuity environment in which the Neuron and the SmartLinx server interface with a relatively smaller number of other sensors and medical devices. In addition, the SmartLinx server may interface with a number of other computer systems, as shown in the example illustrated in FIG. 38, such as one or more Neurons (e.g., via one or more VPN connections), one or more database servers, an Active Directory server, an EMR server, one or more networked medical devices and/or medical devices, and/or other devices and/or systems.

While aspects of the disclosure have been described with respect to specific embodiments, numerous modifications are possible. Thus, although aspects of the disclosure have been described with respect to specific embodiments, it will be appreciated that the disclosure is intended to encompass any and all modifications and equivalents within the scope of the appended claims.

One or more aspects of the disclosure may be embodied in computer-usable data or computer-executable instructions, such as in one or more program modules, executed by one or more computers or other devices to perform the operations described herein. Generally, program modules include routines, programs, objects, components, data structures, and the like that perform particular tasks or implement particular abstract data types when executed by one or more processors in a computer or other data processing device. The computer-executable instructions may be stored on a computer-readable medium such as a hard disk, optical disk, removable storage media, solid-state memory, RAM, and the like. The functionality of the program modules may be combined or distributed as desired in various embodiments. In addition, the functionality may be embodied in whole or in part in firmware or hardware equivalents, such as integrated circuits, application-specific integrated circuits (ASICs), field programmable gate arrays (FPGA), and the like. Particular data structures may be used to more effectively implement one or more aspects of the disclosure, and such data structures are contemplated to be within the scope of computer executable instructions and computer-usable data described herein.

Various aspects described herein may be embodied as a method, an apparatus, or as one or more computer-readable media storing computer-executable instructions. Accordingly, those aspects may take the form of an entirely hardware embodiment, an entirely software embodiment, an entirely firmware embodiment, or an embodiment combining software, hardware, and firmware aspects in any combination. In addition, various signals representing data or events as described herein may be transferred between a source and a destination in the form of light or electromagnetic waves traveling through signal-conducting media such as metal wires, optical fibers, or wireless transmission media (e.g., air or space). In general, the one or more computer-readable media may comprise one or more non-transitory computer-readable media.

As described herein, the various methods and acts may be operative across one or more computing servers and one or more networks. The functionality may be distributed in any manner, or may be located in a single computing device (e.g., a server, a client computer, and the like). For example, in alternative embodiments, one or more of the computing platforms discussed above may be combined into a single computing platform, and the various functions of each computing platform may be performed by the single computing platform. In such arrangements, any and/or all of the above-discussed communications between computing platforms may correspond to data being accessed, moved, modified, updated, and/or otherwise used by the single computing platform. Additionally or alternatively, one or more of the computing platforms discussed above may be implemented in one or more virtual machines that are provided by one or more physical computing devices. In such arrangements, the various functions of each computing platform may be performed by the one or more virtual machines, and any and/or all of the above-discussed communications between computing platforms may correspond to data being accessed, moved, modified, updated, and/or otherwise used by the one or more virtual machines.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps depicted in the illustrative figures may be performed in other than the recited order, and one or more depicted steps may be optional in accordance with aspects of the disclosure.

What is claimed is:

1. A method, comprising:
establishing, by a server computing device, a first network connection to a first bedside computing device;
selecting, by the server computing device, a first device profile for the first bedside computing device;
providing, by the server computing device, the first device profile to the first bedside computing device;
establishing, by the server computing device, a second network connection to a second bedside computing device different from the first bedside computing device;
selecting, by the server computing device, a second device profile for the second bedside computing device; and
providing, by the server computing device, the second device profile to the second bedside computing device.

2. The method of claim 1, wherein the first device profile for the first bedside computing device is selected based on a unique identifier associated with the first bedside computing device.

3. The method of claim 1, wherein the first device profile for the first bedside computing device is selected based on a deployment location of the first bedside computing device.

4. The method of claim 1, wherein the first device profile for the first bedside computing device is configured to cause the first bedside computing device to process sensor data received from at least one sensor that is directly connected to the first bedside computing device.

5. The method of claim 1, wherein the first device profile for the first bedside computing device is configured to cause the first bedside computing device to process sensor data received from at least one sensor that is connected to the first bedside computing device via at least one medical device.

6. The method of claim 1, wherein the first device profile includes location information associated with the first bedside computing device, the location information specifying a deployment location of the first bedside computing device in a healthcare facility.

7. The method of claim 1, wherein the first device profile includes service information associated with the first bedside computing device, the service information specifying at least one healthcare service for which the first device profile has been customized.

8. The method of claim 1, wherein the first device profile includes network information associated with the first bedside computing device, the network information specifying one or more connection settings to be used by the first bedside computing device.

9. The method of claim 1, wherein the first device profile includes access information associated with the first bedside computing device, the access information specifying one or more authorized users of the first bedside computing device.

10. The method of claim 1, wherein the first device profile includes user interface information associated with the first bedside computing device, the user interface information specifying one or more layout settings to be used by the first bedside computing device in presenting one or more graphical user interfaces.

11. The method of claim 1, wherein the first device profile includes application information associated with the first bedside computing device, the application information specifying one or more applications that are executable on the first bedside computing device.

12. A server computing device, comprising:
at least one processor; and
memory storing computer-readable instructions that, when executed by the at least one processor, cause the server computing device to:
establish a first network connection to a first bedside computing device;
select a first device profile for the first bedside computing device;
provide the first device profile to the first bedside computing device;
establish a second network connection to a second bedside computing device different from the first bedside computing device;
select a second device profile for the second bedside computing device; and
provide the second device profile to the second bedside computing device.

13. The server computing device of claim 12, wherein the first device profile for the first bedside computing device is selected based on a unique identifier associated with the first bedside computing device.

14. The server computing device of claim 12, wherein the first device profile for the first bedside computing device is selected based on a deployment location of the first bedside computing device.

15. The server computing device of claim 12, wherein the first device profile for the first bedside computing device is configured to cause the first bedside computing device to process sensor data received from at least one sensor that is directly connected to the first bedside computing device.

16. The server computing device of claim 12, wherein the first device profile for the first bedside computing device is configured to cause the first bedside computing device to process sensor data received from at least one sensor that is connected to the first bedside computing device via at least one medical device.

17. The server computing device of claim 12, wherein the first device profile includes location information associated with the first bedside computing device, the location information specifying a deployment location of the first bedside computing device in a healthcare facility.

18. The server computing device of claim 12, wherein the first device profile includes service information associated with the first bedside computing device, the service information specifying at least one healthcare service for which the first device profile has been customized.

19. The server computing device of claim 12, wherein the first device profile includes network information associated with the first bedside computing device, the network information specifying one or more connection settings to be used by the first bedside computing device.

20. One or more non-transitory computer-readable media storing instructions that, when executed by at least one computing device, cause the at least one computing device to:
establish a first network connection to a first bedside computing device;
select a first device profile for the first bedside computing device;
provide the first device profile to the first bedside computing device;
establish a second network connection to a second bedside computing device different from the first bedside computing device;
select a second device profile for the second bedside computing device; and
provide the second device profile to the second bedside computing device.

21. A method, comprising:
capturing, by a first bedside computing device, sensor data received from one or more sensors connected to the first bedside computing device;
processing, by the first bedside computing device, the sensor data to determine patient vital signs information; and
providing, by the first bedside computing device, the patient vital signs information to a server computing device that is configured to receive vital signs information from one or more additional bedside computing devices different from the first bedside computing device.

22. The method of claim 21, wherein at least a portion of the sensor data is received from at least one sensor that is directly connected to the first bedside computing device.

23. The method of claim 21, wherein at least a portion of the sensor data is received from at least one sensor that is connected to the first bedside computing device via at least one medical device.

24. The method of claim 21, further comprising:
presenting, by the first bedside computing device, at least one graphical user interface that includes at least a portion of the patient vital signs information.

25. The method of claim 21, wherein the server computing device is further configured to provide at least a portion of the patient vital signs information to at least one electronic medical records (EMR) system.

26. The method of claim 21, wherein the first bedside computing device is configured to capture and process the sensor data based on a first device profile for the first bedside computing device that is managed, at least in part, by the server computing device.

27. The method of claim 26, wherein the first device profile includes location information associated with the first bedside computing device, the location information specifying a deployment location of the first bedside computing device in a healthcare facility.

28. The method of claim 26, wherein the first device profile includes service information associated with the first bedside computing device, the service information specifying at least one healthcare service for which the first device profile has been customized.

29. The method of claim 26, wherein the first device profile includes network information associated with the first bedside computing device, the network information specifying one or more connection settings to be used by the first bedside computing device.

30. The method of claim 26, wherein the first device profile includes access information associated with the first bedside computing device, the access information specifying one or more authorized users of the first bedside computing device.

31. The method of claim 26, wherein the first device profile includes user interface information associated with the first bedside computing device, the user interface information specifying one or more layout settings to be used by the first bedside computing device in presenting one or more graphical user interfaces.

32. The method of claim 26, wherein the first device profile includes application information associated with the first bedside computing device, the application information specifying one or more applications that are executable on the first bedside computing device.

33. A first bedside computing device, comprising:
at least one processor; and
memory storing computer-readable instructions that, when executed by the at least one processor, cause the first bedside computing device to:
capture sensor data received from one or more sensors connected to the first bedside computing device;
process the sensor data to determine patient vital signs information; and
provide the patient vital signs information to a server computing device that is configured to receive vital signs information from one or more additional bedside computing devices different from the first bedside computing device.

34. The first bedside computing device of claim 33, wherein at least a portion of the sensor data is received from at least one sensor that is directly connected to the first bedside computing device.

35. The first bedside computing device of claim 33, wherein at least a portion of the sensor data is received from at least one sensor that is connected to the first bedside computing device via at least one medical device.

36. The first bedside computing device of claim 33, wherein the memory stores additional computer-readable instructions that, when executed by the at least one processor, further cause the first bedside computing device to:
present at least one graphical user interface that includes at least a portion of the patient vital signs information.

37. The first bedside computing device of claim 33, wherein the first bedside computing device is configured to capture and process the sensor data based on a first device profile for the first bedside computing device that is managed, at least in part, by the server computing device.

38. The first bedside computing device of claim 37, wherein the first device profile includes location information associated with the first bedside computing device, the location information specifying a deployment location of the first bedside computing device in a healthcare facility.

39. The first bedside computing device of claim 37, wherein the first device profile includes service information associated with the first bedside computing device, the service information specifying at least one healthcare service for which the first device profile has been customized.

40. One or more non-transitory computer-readable media storing instructions that, when executed by a first bedside computing device, cause the first bedside computing device to:
capture sensor data received from one or more sensors connected to the first bedside computing device;
process the sensor data to determine patient vital signs information; and
provide the patient vital signs information to a server computing device that is configured to receive vital signs information from one or more additional bedside computing devices different from the first bedside computing device.

41. A method, comprising:
receiving, by a server computing device, input defining a first device profile for a first set of one or more bedside computing devices;
establishing, by the server computing device, a network connection to at least one bedside computing device included in the first set of one or more bedside computing devices; and
providing, by the server computing device, the first device profile to the at least one bedside computing device.

42. The method of claim 41, wherein the server computing device is configured to interface with at least one electronic medical records (EMR) system for logging of patient vital signs information received from the first set of one or more bedside computing devices.

43. The method of claim 41, wherein the server computing device is configured to interface with at least one central monitoring system for live monitoring of patient vital signs information received from the first set of one or more bedside computing devices.

44. The method of claim 41, wherein the server computing device is configured to interface with at least one directory system for identifying one or more users of the one or more bedside computing devices.

45. The method of claim 41, wherein the one or more bedside computing devices are deployed in a patient care environment associated with a healthcare facility.

46. The method of claim 41, further comprising:
receiving, by the server computing device, input defining an updated device profile for a second set of one or more bedside computing devices;
establishing, by the server computing device, a network connection to at least one second bedside computing device included in the second set of one or more bedside computing devices; and
providing, by the server computing device, the updated device profile to the at least one second bedside computing device.

47. The method of claim 46, wherein providing the updated device profile to the at least one second bedside computing device includes providing a firmware update to the at least one second bedside computing device.

48. The method of claim 41, wherein the first device profile includes location information associated with the first set of one or more bedside computing devices, the location information specifying a deployment location of the first set of one or more bedside computing devices in a healthcare facility.

49. The method of claim 41, wherein the first device profile includes service information associated with the first set of one or more bedside computing devices, the service information specifying at least one healthcare service for which the first device profile has been customized.

50. The method of claim 41, wherein the first device profile includes network information associated with the first set of one or more bedside computing devices, the network information specifying one or more connection settings to be used by the first set of one or more bedside computing devices.

51. The method of claim 41, wherein the first device profile includes access information associated with the first set of one or more bedside computing devices, the access information specifying one or more authorized users of the first set of one or more bedside computing devices.

52. The method of claim 41, wherein the first device profile includes user interface information associated with the first set of one or more bedside computing devices, the user interface information specifying one or more layout settings to be used by the first set of one or more bedside computing devices in presenting one or more graphical user interfaces.

53. The method of claim 41, wherein the first device profile includes application information associated with the first set of one or more bedside computing devices, the application information specifying one or more applications that are executable on the first set of one or more bedside computing devices.

54. A server computing device, comprising:
at least one processor; and
memory storing computer-readable instructions that, when executed by the at least one processor, cause the server computing device to:
receive input defining a first device profile for a first set of one or more bedside computing devices;
establish a network connection to at least one bedside computing device included in the first set of one or more bedside computing devices; and
provide the first device profile to the at least one bedside computing device.

55. The server computing device of claim 54, wherein the server computing device is configured to interface with at least one electronic medical records (EMR) system for logging of patient vital signs information received from the first set of one or more bedside computing devices.

56. The server computing device of claim 54, wherein the server computing device is configured to interface with at least one central monitoring system for live monitoring of patient vital signs information received from the first set of one or more bedside computing devices.

57. The server computing device of claim 54, wherein the server computing device is configured to interface with at least one directory system for identifying one or more users of the one or more bedside computing devices.

58. The server computing device of claim 54, wherein the memory stores additional computer-readable instructions that, when executed by the at least one processor, further cause the server computing device to:
receive input defining an updated device profile for a second set of one or more bedside computing devices;
establish a network connection to at least one second bedside computing device included in the second set of one or more bedside computing devices; and
provide the updated device profile to the at least one second bedside computing device.

59. The server computing device of claim 58, wherein providing the updated device profile to the at least one second bedside computing device includes providing a firmware update to the at least one second bedside computing device.

60. One or more non-transitory computer-readable media storing instructions that, when executed by at least one computing device, cause the at least one computing device to:
receive input defining a first device profile for a first set of one or more bedside computing devices;
establish a network connection to at least one bedside computing device included in the first set of one or more bedside computing devices; and
provide the first device profile to the at least one bedside computing device.
